(19) United States
(12) Patent Application Publication
(10) Pub. No.: US 2023/0245719 A1
(43) Pub. Date: Aug. 3, 2023

(54) Method To Identify Disease LInked Genetic Fusions

(71) Applicant: ISTITUTO ROMAGNOLO PER LO STUDIO DEI TUMORI "DINO AMADORI" - IRST IRCCS, Meldola (FC) (IT)

(72) Inventors: Giovanni MARTINELLI, Meldola (FC) (IT); Anna FERRARI, Meldola (FC) (IT)

(73) Assignee: ISTITUTO ROMAGNOLO PER LO STUDIO DEI TUMORI "DINO AMADORI" - IRST IRCCS, Meldola (FC) (IT)

(21) Appl. No.: 18/001,335

(22) PCT Filed: Jun. 10, 2021
(86) PCT No.: PCT/EP2021/065692

§ 371 Date: Dec. 9, 2022

(30) Foreign Application Priority Data
Jun. 11, 2020 (EP) 20179583.8

(51) Int. Cl. G16B 30/00; G16B 20/00; G16H 50/20

(52) U.S. Cl. CPC G16B 30/00; G16B 20/00; G16H 50/20

(57) ABSTRACT

The present invention refers to a method to identify a genetic fusion associated with a subject affected by a disease, preferably B-cell acute lymphoblastic leukemia (B-ALL). A method to classify a subject affected by a disease into a known subtype of said disease and methods to select a suitable therapeutic treatment involving the identification of genetic fusions in said subject are also disclosed.

### TECHNICAL FIELD

The present invention refers to a method to identify a genetic fusion associated with a subject affected by a disease, preferably B-cell acute lymphoblastic leukemia (B-ALL), and to a method to classify an adult B-ALL subject that is negative for t(9;22), t(4;11) and t(1;19) translocations (Ph−/−/− B-ALL subjects) into a known B-ALL subgroup.

### BACKGROUND ART

Some crucial cancer related genes are well known to be promiscuous in fusion generation. These genes rearrange (e.g. MLL, ABL1, NTRK1/2/3, ALK, RET) with different partner genes, not all already discovered. Many of these gene fusions have diagnostic, prognostic and therapeutic relevance and their identification is pivotal in cancer.

Diagnostic fusion transcript identification is currently limited to few translocations with well-known clinical relevance, missing the identification of novel or rare gene fusion, even if important for clinicians support.

In particular, B-cell acute lymphoblastic leukemia is an aggressive hematological tumor characterized by the proliferation of undifferentiated B-cell precursors. B-ALL patients are routinely classified into different subgroups based on the identification of peculiar genomic alterations[1]. The classification of B-ALL into subgroups is crucial for the definition of the patient's clinical course and for patients' outcome, due to the availability of genomic based-target therapies. In the past decades, the identification of the fusion gene BCR-ABL1 in Philadelphia-positive (Ph+) B-ALL patients followed by the development of selective inhibitors (tyrosine kinase inhibitors, TKi) has dramatically changed the prognosis of these patients, by transforming a high risk ALL subtype into a manageable disease [2,3].

According to conventional diagnostic methodologies, around 40% of adult B-ALL patients are grouped in three main subtypes: 1) the above mentioned Ph+B-ALL with t(9;22)(q34;q11) translocation; ii) MLL-AFF1 positive B-ALL characterized by t(4;11)(q21;q23) translocation; iii) TCF3-PBX1 positive B-ALL carrying t(1;19)(q23;p13) translocation, which represent 23.2%, 11.9% and 2.9% of adult B-ALL patients, respectively[4,5]. In addition, the translocation t(12;21)(p13;q22)-ETV6-RUNX1, which is highly represented in the pediatric cohort, in adult patients reaches 1% [4,5]. The remaining 61% of Philadelphia negative B-ALL adult patients, labeled as “B-Other ALL”, is a highly heterogeneous leukemia subgroup. Recently, the effort of different researchers in the characterization of this subgroup resulted in the identification of the Philadelphia-like [6] and other subgroups [7-14]. Moreover, genome-wide sequencing and RNA-sequencing (RNA-seq) of adult and pediatric ALL samples has shown that many of the “B-Other ALL” cases harbor peculiar genetic aberrations and, in particular, different translocations [1,15].

Acute lymphoblastic and myeloid leukemia have subgroups characterized by promiscuous gene fusions (e.g. MLL, ABL1/2, JAK2, ZNF384).

The MLL (mixed-lineage leukemia; or KMT2A) gene is involved in chromosomal translocations in a subtype of acute leukemia, which represents approximately 10% of acute lymphoblastic leukemia and 2.8% of acute myeloid leukemia cases. These translocations form fusions with various genes, of which more than 80 partner genes for MLL have been identified. The most recurrent fusion partner in MLL rearrangements (MLL-r) is AF4, accounting for approximately 36% of MLL-r leukemia and particularly prevalent in MLL-r acute lymphoblastic leukemia (ALL) cases (57%). MLL-r leukemia is associated with a sudden onset, aggressive progression, and notoriously poor prognosis in comparison to non-MLL-r leukemias. Despite modern chemotherapeutic interventions and the use of hematopoietic stem cell transplantations, infants, children, and adults with MLL-r leukemia generally have poor prognosis and response to these treatments [96]. There is a clear clinical need for a new effective therapy. New clinical trials are ongoing to evaluate some new therapeutic options for MLL-r leukemias, for example clinical trials with different drugs such as Menin, DOT1L Inhibitors can be retrieved from https://clinicaltrials.gov/.

Rarer or new MLL rearrangements are poorly investigated for diagnostic purposes and not evaluable samples for cytogenetic test (for MLL-r identifications) are usually not further investigated (excluding t(4;11) by RT-PCR).

Also ABL1 and ABL-class genes (ABL1, ABL2, CSF1R, and PDGFRB) have different gene partners. Regarding treatment choices, this approach could also already applied to B-ALL Ph-like patients, a high-risk subtype characterized by genomic alterations that activate cytokine receptor and kinase signaling. Kinase more frequent alterations include fusions involving ABL-class genes (ABL1, ABL2, CSF1R, and PDGFRB) sensitive to ABL1 tyrosine kinase inhibitors (TKIs) or rearrangements that create JAK2 fusion proteins or truncating rearrangements of the erythropoietin receptor (EPOR) that are sensitive to ruxolitinib in vitro [97]. ABL-class and JAK genes are also promiscuous genes, and their fusion detection is crucial for classification and for alternative therapeutic option identification.

Ph-like clinical trials) with different inhibitors that target Ph-like markers are disclosed in https://clinicaltrials.gov/. In this scenario, the introduction of RNA-seq in the routine diagnostic practice would significantly improve the sub-classification of ALL and other cancer disease and the identification of prognostic and therapeutic markers [13,14].

However, the use of RNA-seq for clinical purposes is still very challenging due to heterogeneity and the complexity of data analysis, with a large amount of predicted fusions and false positive calls difficult to manage, validate and clinically evaluate.

To overcome this problem, different studies have compared and combined different bioinformatics pipelines in order to establish the best strategy for the detection of true positive fusion transcripts in different lymphoid malignances [16,17].

However, robust bioinformatic pipelines for the identification of fusion genes are not yet available.

Here, the inventors investigated the efficacy of a capture-based RNA-seq panel (1385 genes) to characterize a heterogeneous group of adult B-Other ALL patients, negative for t(9;22)(q34;q11), t(4;1)(q21;q23) and t(1;19)(q23;p13) translocations according to conventional methodologies.

Our cohort, hereafter referred to as “Ph−/−/−” B-ALL, was investigated for the presence of gene fusions with a in house RNA-seq pipeline integrating four different fusion-mining tools with an exhaustive B-ALL fusion database.

Inventors also identified, starting from low amount good quality RNA and applying the method of the invention, rare/novel MLL and other genes transcripts in a subset of acute leukaemia not otherwise evaluated.

This paves the way to alternative treatments otherwise precluded for these patients.

### SUMMARY OF THE INVENTION

Until few years ago, about 60% of adult patients with B-cell acute lymphoblastic leukemia (B-ALL) were classified as “B-Other ALL” due to the absence of known genetic alterations in conventional diagnostic assays. Recently, transcriptomic profile revealed novel genetic subgroups defined by specific fusion transcripts, gene expression profiles and/or sequence mutations (e.g. MEF2D, ZNF384, DUX4, BCL2-MYC, NUTM1, HLF-rearranged and PAX5-driven subtypes). B-Other is not routinely screened for fusions but novel findings strongly suggest that a RNA-sequencing approach is needed even if still challenging for genetic complexity, low subtype/fusion frequency and data analysis.

To genetically characterize 63 adult B-ALL cases negative for common t(9;22), t(4;11) and t(1;19) translocations (hereafter referred as Ph−/−/− B-ALL), the inventors performed a capture-based RNA-sequencing panel featuring 1385 genes and they optimized a bioinformatic pipeline to accurately detect known and novel gene fusions.

Ph−/−/− B-ALL cases are characterized by a high rate of fusion transcripts. The inventors identified 65 fusion transcripts in 41 out of 63 samples (65.1%) by combining four fusion-mining tools (STAR-Fusion, Manta, FusionCatcher and TopHat-Fusion) and by filtering fusions based on prior literature in B-ALL. The inventors identified 22 novel fusion transcripts in 33.8% of cases, such as THADA-CDH1, TET3-ETV6 and NUMA1-CSF1R in Ph-like cases. Notably, our approach allowed to correctly assign 33.3% of cases to a known B-ALL subtypes (e.g. ZNF384, rare KMT2A, BCL2/MYC-rearranged). Most of the fusion transcripts were expressed and included actionable/druggable targets.

The present results demonstrate that Ph−/−/− B-ALL is associated with a number of new and rare translocations that in some cases could generate druggable fusion transcripts, rapidly and precisely detected by a powerful bioinformatics pipeline, and pave the way to a more precise prognostic and therapeutic classification.

The invention provides a method to identify at least one genetic fusion in the genome of a subject affected by a disease comprising the following steps:

a) obtaining genomic raw sequencing data from a sample isolated from the subject,

b) analyzing said data with at least three informatic tools able to identify genetic fusions from said genomic sequencing data thereby obtaining a first genetic fusion list comprising fusions identified by at least one of said tools,

c) selecting genetic fusions from said first genetic fusion list, being detected by at least three of said tools used in step b) thereby obtaining a second genetic fusion list,

d) selecting genetic fusions from said first genetic fusion list being detected by one or two of said tools used in step b) and adding them to said second genetic fusion list provided that they meet at least one of the following criteria:
d1. genetic fusions are known for said disease,
d2. for fusions detected by two different tools but not known for said disease, fusion is not marked “false positive” by anyone of said tools used in b),
d3. for fusions detected by two tools, the fusion is labeled as significant for at least one of the three following events: a) positive score in a tool combined with read/s positivity in the other tool, b) fusion positive comments in the output of a tool, c) EBF1 and ERG genes read-throughs,

and optionally

e) comparing the fusions present in said obtained second genetic fusion list to at least one database of genetic fusions in order to obtain an annotated fusion list wherein for each fusion it is annotated if said fusion is known in other diseases and/or in normal samples.

In an embodiment, the invention provides a method to identify at least one genetic fusion in the genome of a subject affected by a disease comprising the following steps:
a) obtaining genomic raw sequencing data from a sample isolated from the subject,
b) analyzing said data with the following tools: Fusion Catcher, STAR-Fusion, RNA-Seq Alignment and TopHat Alignment, thereby obtaining a first genetic fusion list comprising fusions identified by at least one of said tools,
c) selecting genetic fusions from said first genetic fusion list, being detected by at least three of tools as in b) thereby obtaining a second genetic fusion list,
d) selecting genetic fusions from said first genetic fusion list being detected by one or two of tools as in b) and adding them to said second genetic fusion list provided that they meet at least one of the following criteria:
   d1. genetic fusions are known for said disease,
   d2. for fusions detected by two different tools but not known for said disease, fusion is not marked “false positive” by the tool Fusion Catcher,
   d3. for fusions detected by two tools, the fusion is labeled as significant for at least one of the three following events: a) Manta positive score combined with read/s positivity in the other tool, b) fusion positive comments in “FusionCatcher summary candidate fusion” output, c) EBF1 and ERG gene read-throughs in FusionCatcher,

and optionally
e) comparing the fusions present in said obtained second genetic fusion list to at least one genetic fusion database selected from i) tumor fusion gene data portal (https://www.tumorfusions.org/), ii) COSMIC (https://cancer.sanger.ac.uk/osmic/fusion), iii) ChimerKB (http://www.kobic.re.kr/chimerdb/chimerkb), iv) Mitelman Database of Chromosome Aberrations and Gene Fusions in Cancer (https://mitelmandatabase.isb-cgc.org/mb_search), v) Fusion Gene annotation DataBase (https://ccsm.uth.edu/FusionGDB/), in order to obtain an annotated fusion list wherein for each fusion it is annotated if said fusion is known in other diseases and/or in normal samples.

Preferably the disease is a cancer, preferably a solid or hematological cancer, preferably B-ALL.

The invention further provides a method to help to classify a subject affected by a disease into a known subtype of said disease comprising using the method above disclosed.

In particular, the invention provides a method help to classify an adult B-ALL subject that is negative for t(9;22), t(4;11) and t(1;19) translocations (Ph−/−/− B-ALL subjects) into a known B-ALL comprising using the method above.

The invention further provides a method to select a therapeutic treatment for a subject affected by a disease comprising carrying out the method of the invention in a sample from said subject in order to obtain a list of genetic fusions from said subject and selecting a suitable treatment based on said list.

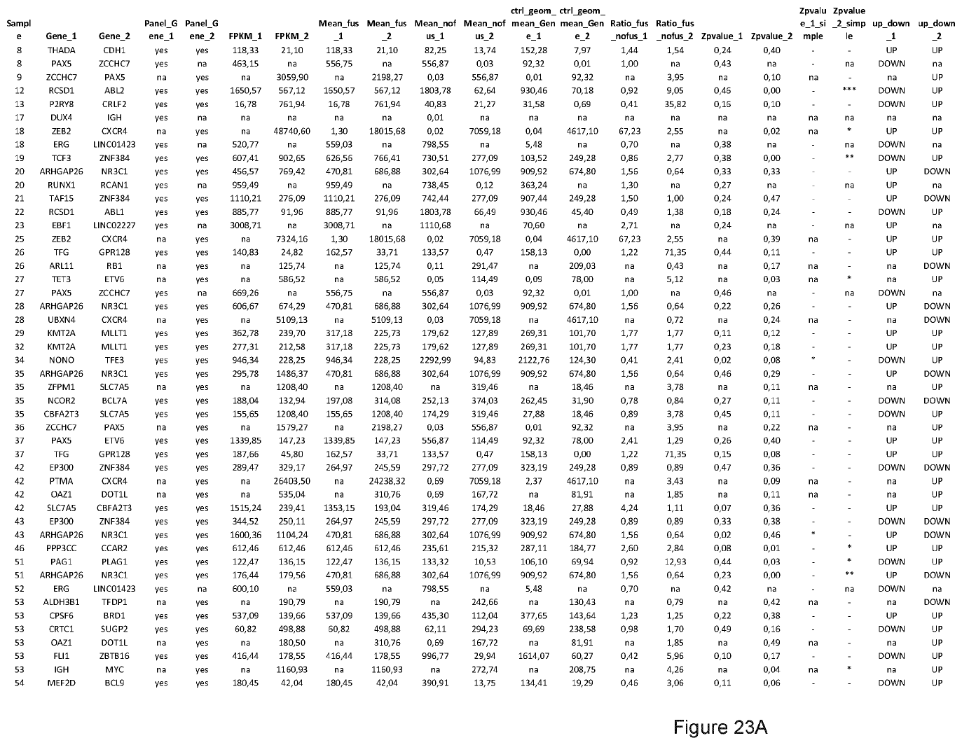

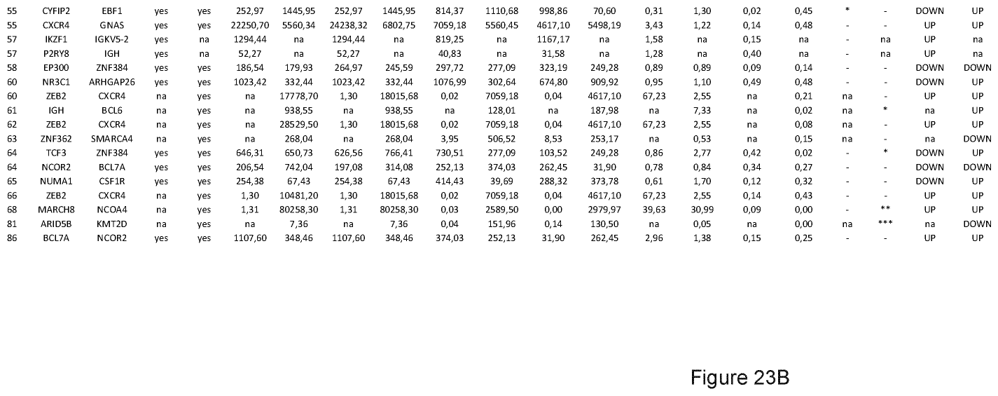

In particular, the invention also provides a method to select a therapeutic treatment for an adult B-cell acute lymphoblastic leukemia (B-ALL) subject that is negative for t(9;22), t(4;11) and t(1;19) translocations (Ph−/−/− B-ALL subject) comprising detecting in a sample of the subject the presence of at least one genetic fusion selected from the group consisting of the fusions indicated in any one of the table 1, 4, or 5 or in FIG. 23.

The invention also provides a method of treatment and/or prevention of B-cell acute lymphoblastic leukemia comprising administering in a subject having a genetic fusion identified according to the method of the invention at least one inhibitor as reported in table 5.

The invention also provides an inhibitor as reported in table 5 for use for the treatment and/or prevention of B-cell acute lymphoblastic leukemia in a subject having a genetic fusion identified according to the method of the invention.

The invention will be illustrated by means of non-limiting examples in reference to the following figures.

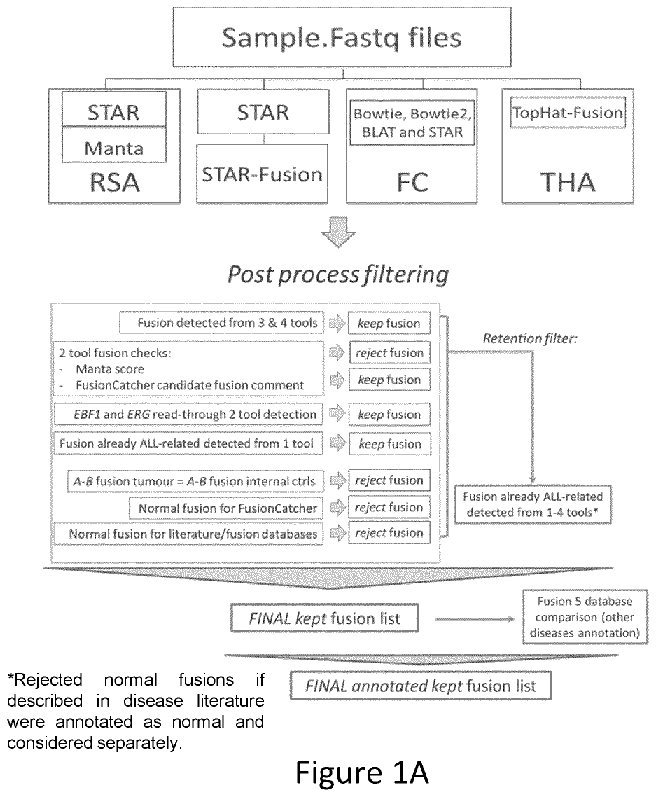

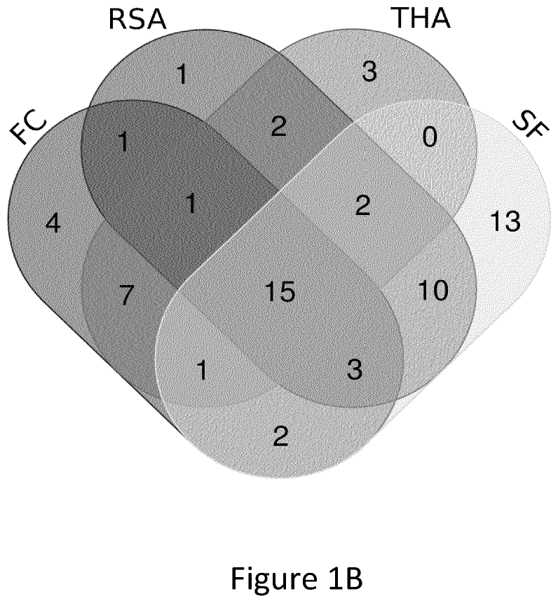

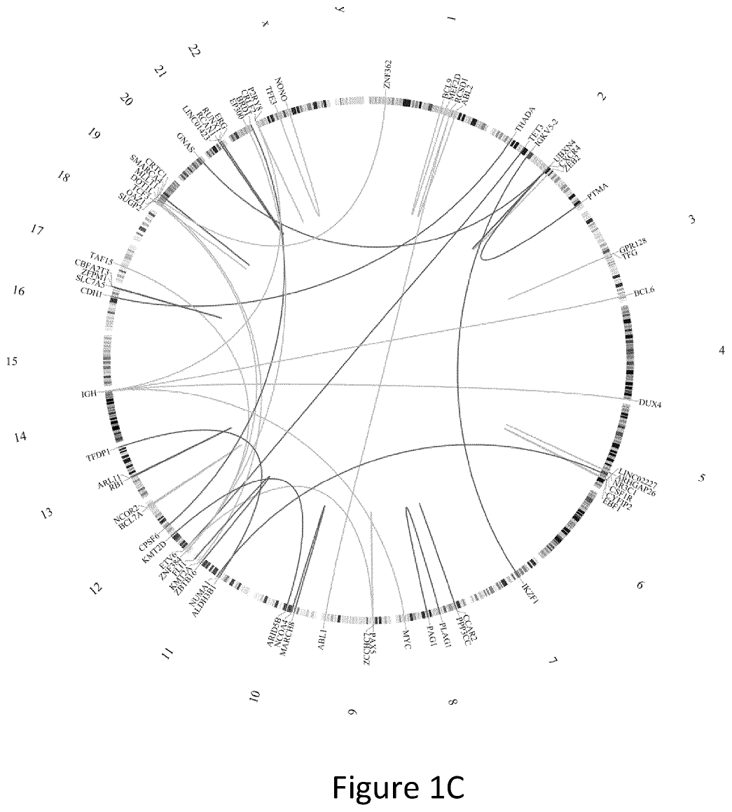

FIG. 1: Fusion detection strategy, mining-tool relationship and detected fusion transcripts in Ph−/−/− B-ALL patients. A) Schematic representation of the overall process of fusion selection and filtering strategy of an exemplative embodiment of the method of the invention; B) Venn diagram showing the overlapping between the identified fusions and their detection by four different bioinformatics tools (RSA: RNA-Seq Alignment; THA: TopHat Alignment; FC: FusionCatcher and SF: STAR-Fusion); C) Circos plot (Circos 0.69.8)[46] depicts the final list of fusions found in 63 Ph−/−/− samples of adult B-ALL. Blue links represent novel fusions, while grey links known ones.

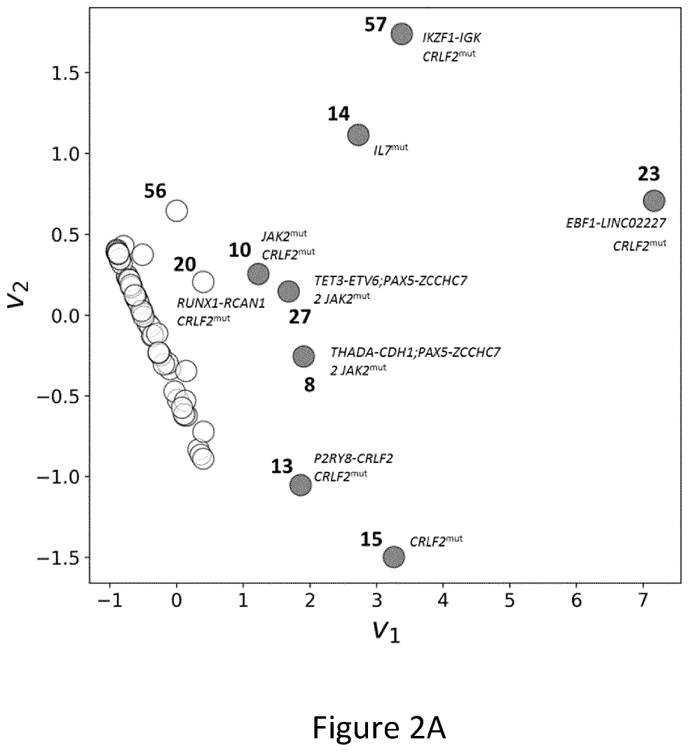

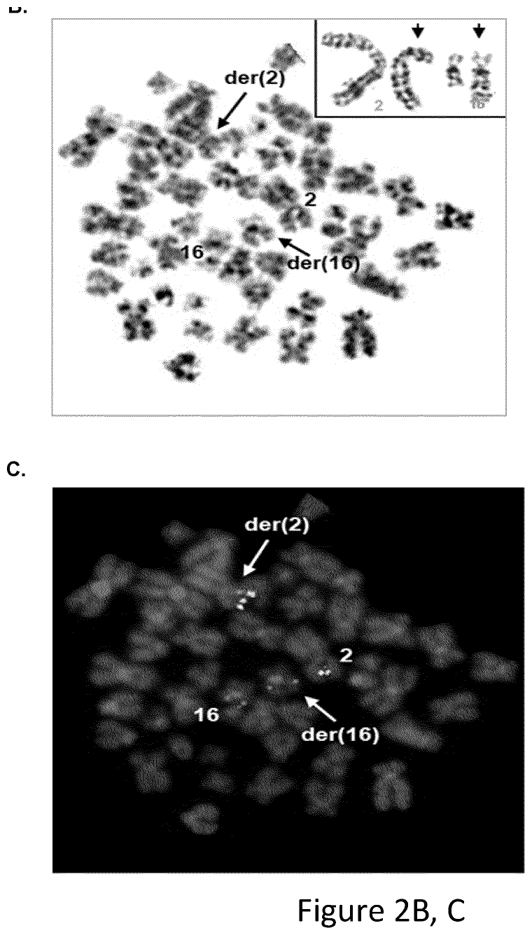

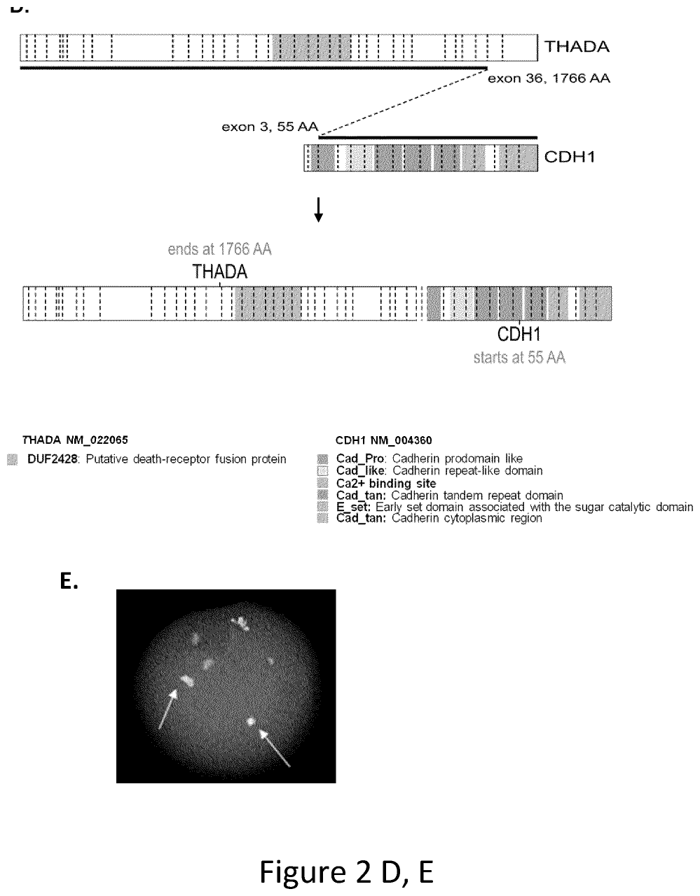

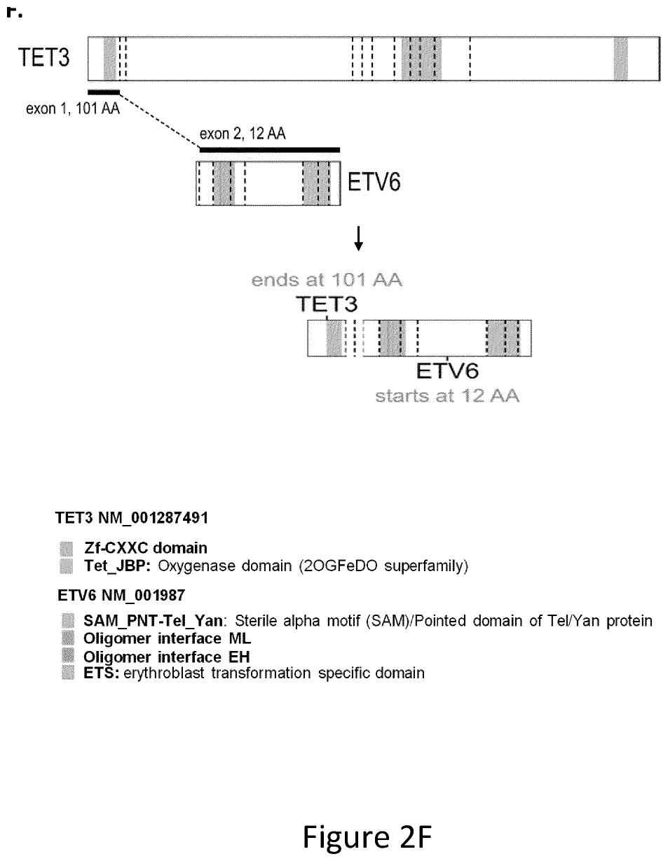

FIG. 2: B-ALL patients harbor novel fusion transcripts associated with Ph-like genetic alterations and characterization of the novel fusion transcripts: THADA-CDH1 and TET3-ETV6. A) K-means clustering of two components two dimensional PCA of a previously identify gene expression signature to identify Ph-like patients[26] (red dots). v1 and v2 represent the first two principal component of PCA applied to expression data. Ph-like associated molecular features are added next to the correspondent sample. mut=mutated. B) GAW-banded metaphase showing t (2;16)(p21;q22) identified in sample 8 (pt #1) by CBA. At top-right, a partial karyotype is shown. C) FISH analysis performed on the same metaphase with RP11-17L5 marked in green, spanning the breakpoint on THADA gene, and RP11-354N7 marked in orange, spanning the breakpoint on CDH1 gene, showed THADA-CDH1 rearrangement on derivative chromosome 2 (one fusion signal). The loss of one expected green signal on derivative chromosome 16 suggests a partial deletion of THADA. D) Schematic representation of THADA-CDH1 fusion. In the upper part of the cartoon, the THADA gene is represented in blue with no functional domain highlighted. The CDH1 gene is represented in red and starting from the N-terminal are represented the following functional domains: the signal peptide (yellow), the precursor peptide (pink), the intracellular domain (red),the transmembrane domain (TM, light orange) and the cytoplasmic domain (dark orange). In the lower part of the cartoon is represented the hypothetic fusion protein THADA-CDH1. E) FISH performed on interphase nucleus of case 27 at relapse with RP11-980B20 marked in orange, spanning the breakpoint on TET3 gene, and Vysis LSI ETV6 (TEL)/RUNX1 (AML1) ES Dual Color Translocation Probe, with LSI ETV6 marked in green. The presence of two fusion signals indicate TET3-ETV6 and ETV6-TET3 rearrangements. The arrows indicate the derivative chromosomes and the fusion signals. F) Schematic representation of TET3-ETV6 fusion. In the upper part of the cartoon, the TET3 gene is represented in blue and, starting from the N-terminal, are represented the following functional domains: the CXXC domain (green) and the CD domain containing the Cys-rich (dark blue) and DSBH regions (light blue). The ETV6 gene is represented in red and starting from the N-terminal are represented the following functional domains: the hetero/homodimerization domain (HLH, light orange), the central domain (dark orange) and the ETS domain (red). In the lower part of the cartoon is represented the hypothetic fusion protein TET3-ETV6.

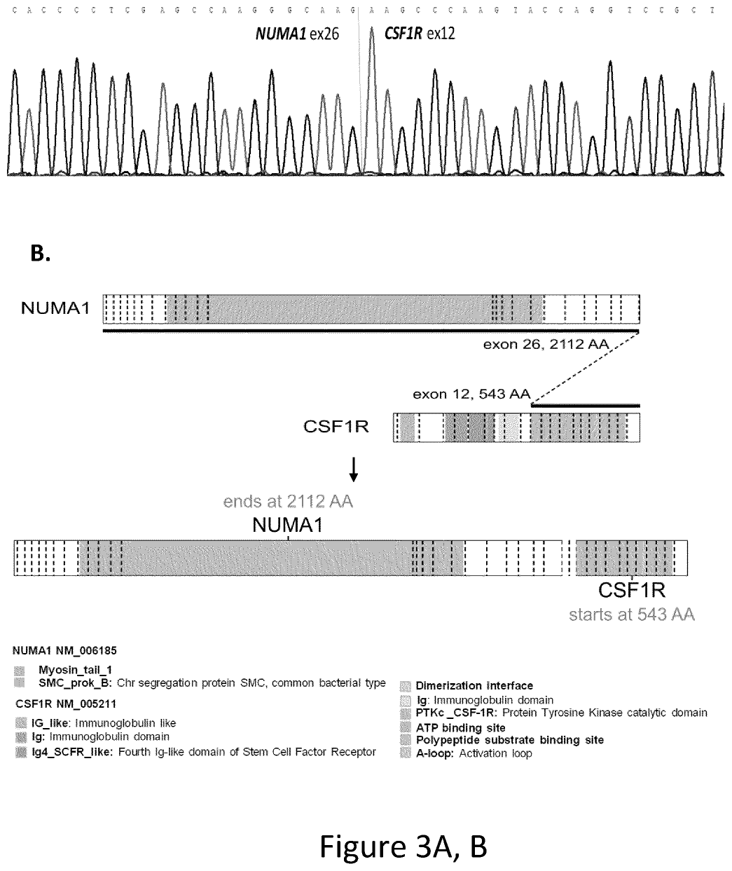

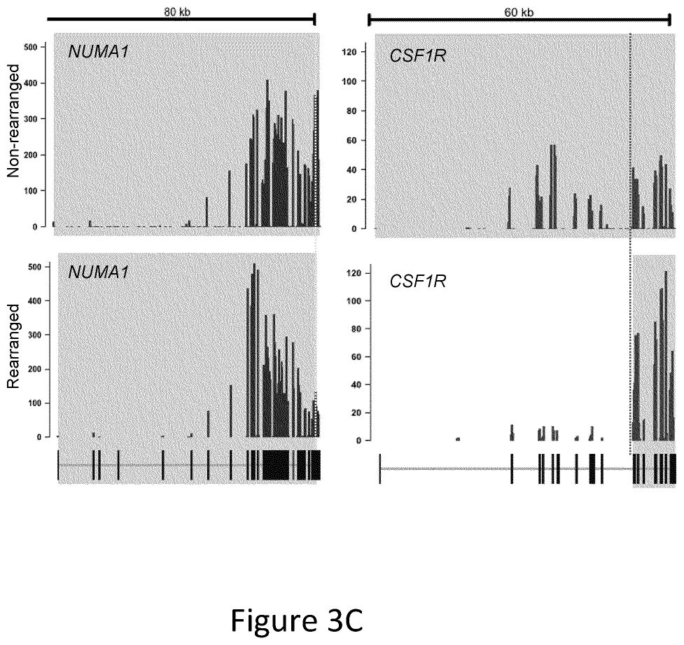

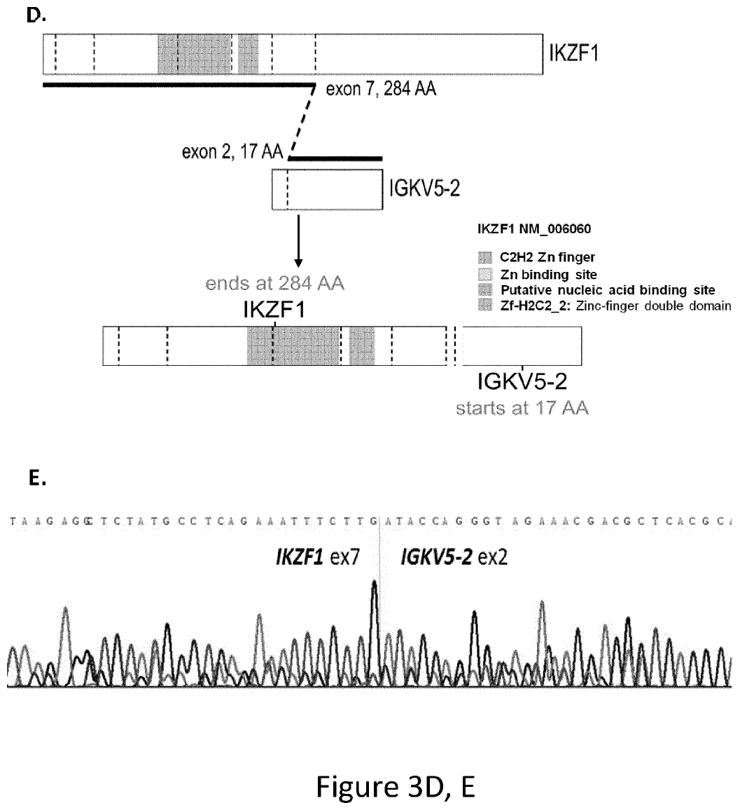

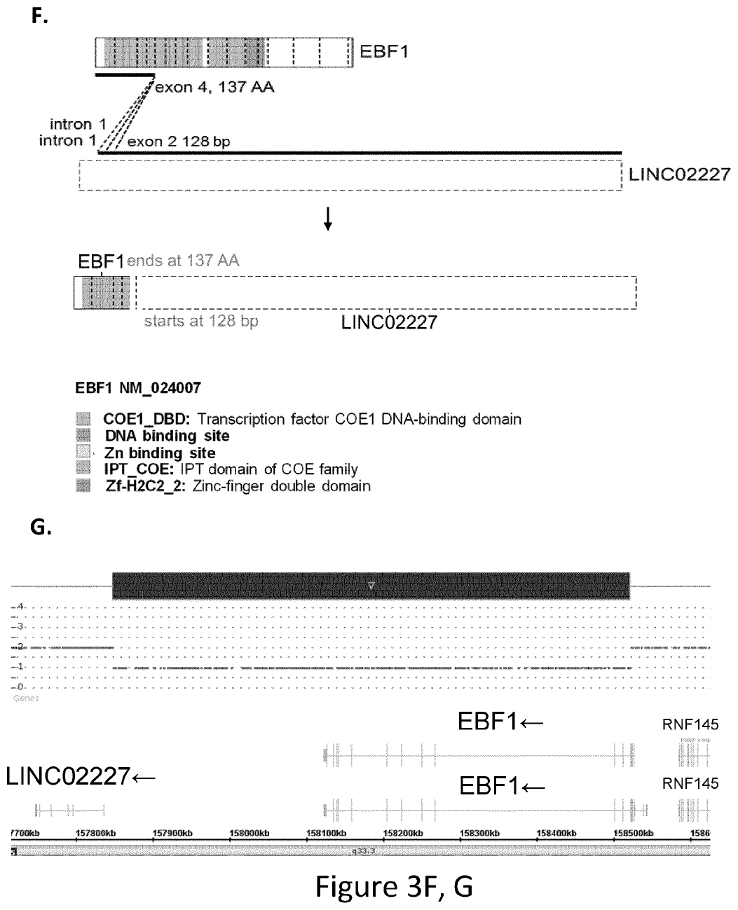

FIG. 3: Identification of the novel transcripts: NUMA1-CSF1R IKZF1-IGKV5-2 and EBF1-LINC02227: A) Sanger sequencing showing NUMA1-CSF1R fusion breakpoint (sample 65). B) Schematic representation of in frame NUMA1-CSF1R fusion. NUMA1 and CSF1R protein diagrams, domain annotations and in frame fusion scheme between NUMA1 exon 26 and CSF1R exon 12 (NUMA1, NM_006185, chr11:71714933, −; CSF1R, NM_005211, chr5:149441412, −; https://proteinpaint.stjude.org/, Human hg19). C) CSFR1 and NUMA1 average read depths across samples with no fusions involving such genes (upper images), compared with read depths of sample 65's mutual fused CSF1R and NUMA1 (lower images). The inventors report these read depths from RNA-sequencing PanCancer Panel along the forward strand. Grey background represents retained part of fusion gene partners in samples with the fusion (Rearranged) and in all other Ph−/−/− non fused samples (Non-Rearranged). D) Schematic representation of in frame IKZF1-IGKV5-2 fusion. IKZF1 and IGKV5-2 protein diagrams, domain annotations and in frame fusion scheme between IKZF1 exon 7 and IGKV5-2 exon 2 (IKZF1, NM_006060, chr7:50459561, +; IGKV5-2, ENST00000390244, chr2: 89197005, +; https://proteinpaint.stjude.org/, Human hg19). E) Sanger sequencing showing IKZF1-IGKV5-2 fusion breakpoint (sample 57). F) Schematic representation of EBF1-LINC02227 fusions. EBF1 and LINC02227 protein diagrams, domain annotations and in frame fusion scheme between EBF1 exon 4 and LINC02227 exon 2 or intron 1 (breakpoint 1: EBF1, NM_024007, chr5:158522628, −; LUNC02227, ENST00000619068, chr5: 157796618, −; breakpoint 2: EBF1, NM_024007, chr5:158522628, −; LUNC02227, ENST00000619068, chr5: 157820245, −; breakpoint 3: EBF1, NM_024007, chr5:158522628, −; LUNC02227, ENST00000619068, chr5: 157823139, −; https://proteinpaint.stjude.org/, Human hg19). G) Copy number status at chromosome 5q33.3 EBF1 and LINC02227 locus. Red rectangle represents heterozygous deletion. Modified from Chromosome Analysis Suits software (Thermo Fisher Scientific) output figure.

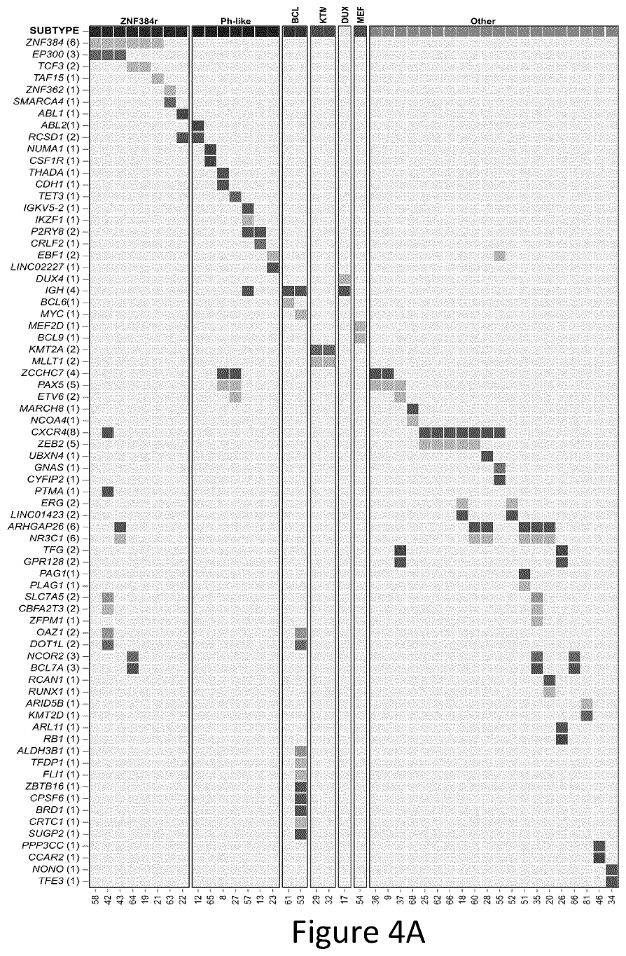

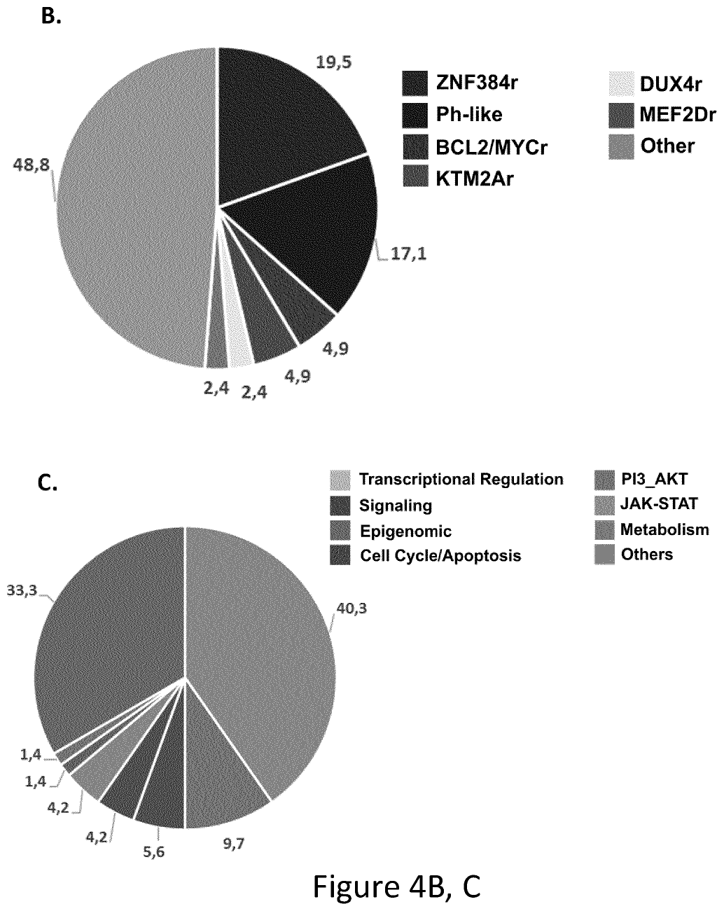

FIG. 4: All genes involved in fusion events in 41/63 Ph−/−/− B-ALL samples. A) Heatmap of all 72 detected fusion partner genes across seven Ph− subtypes. B) Pie chart of subtype frequency in our fused Ph−/−/− B-ALL cohort. C) Pie chart of frequency of selected pathways/functional subgroups.

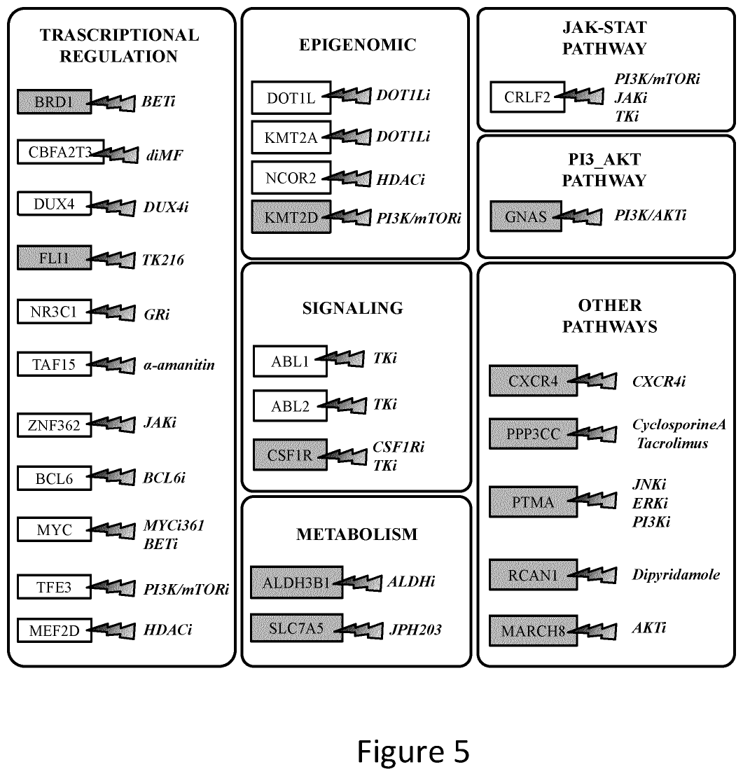

FIG. 5: Graphic representation of known protein inhibitors targeting specific genes involved in the identified fusions. In the figure “i” represents inhibitor and the genes involved in novel fusions are highlighted in light blue. In the picture JAK=janus kinase, TK=tyrosine kinase, HDAC=histone deacetylase, BET=bromodomain and extra-terminal motif, ERK=extracellular signal-regulated kinase, PI3K=Phosphatidylinositol-4,5-Bisphosphate 3-Kinase, mTOR=mechanistic target of rapamycin kinase, CSF1R=colony stimulating factor 1 receptor, DOTL1=DOT1 like histone lysine methyltransferase, DUX4=Double Homeobox 4.

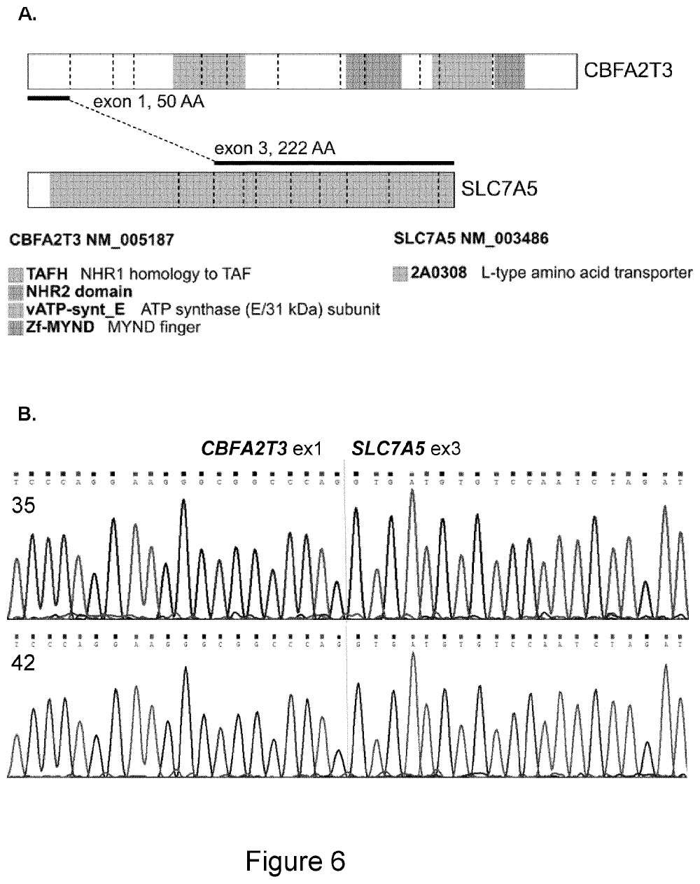

FIG. 6. CBFA2T3-SLC7A5 fusions: A) CBFA2T3 and SLC7A5 protein diagrams, domain annotations and in frame fusion scheme between CBFA2T3 exon 1 and SLC7A5 exon 3 (CBFA2T3, NM_005187, chr16: 89043065, −; SLC7A5, NM_003486, chr16: 87874761, −; https://proteinpaint.stjude.org/, Human hg19). B) CBFA2T3 exon 1-SLC7A5 exon 3 fusion Sanger sequencing chromatogram (samples 35 and 42).

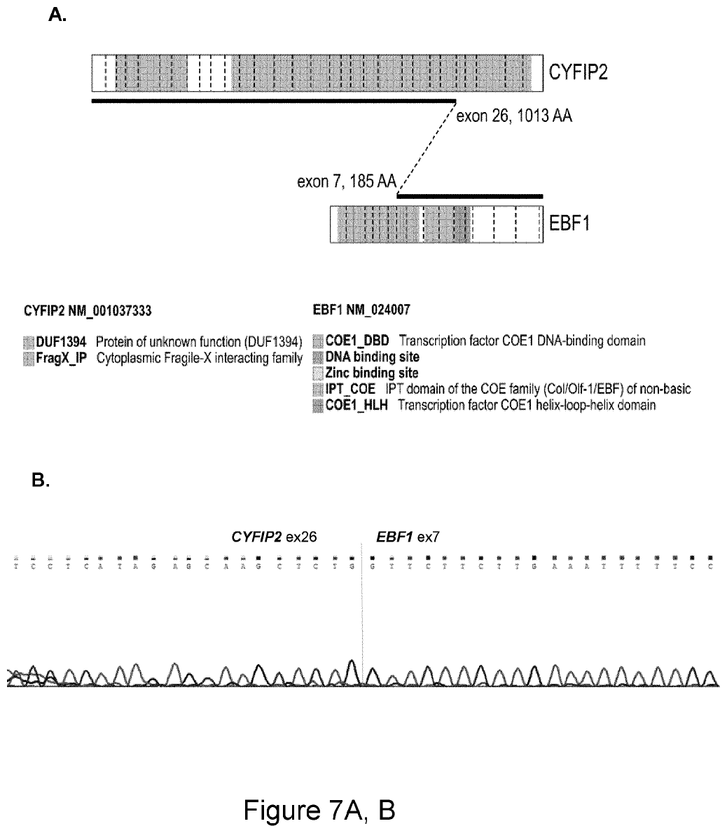

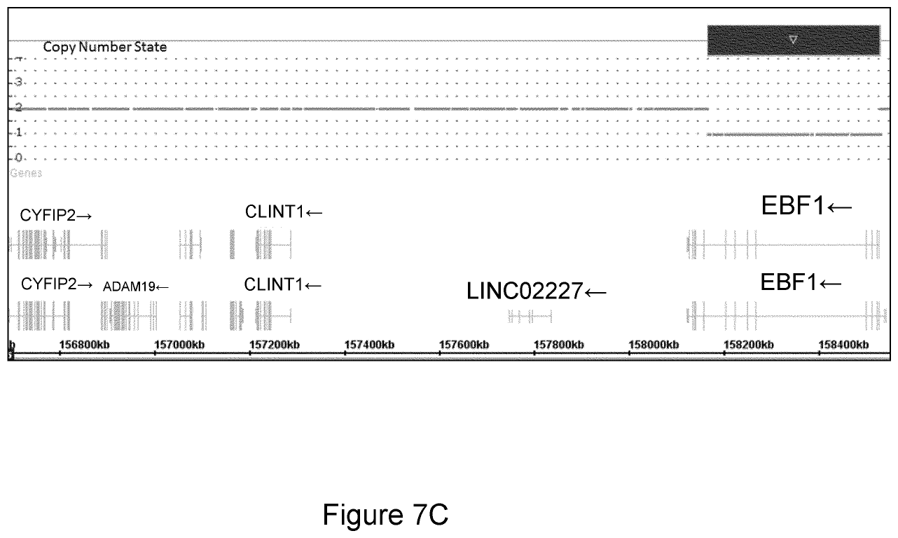

FIG. 7. CYFIP2-EBF1 fusion: A) CYFIP2 and EBF1 protein diagrams, domain annotations and in frame fusion scheme between CYFIP2 exon 26 and EBF1 exon 7 (CYFIP2, NM_001037332, chr5: 156788606, +; EBF1, NM_024007, chr5: 158500472 55, −; https://proteinpaint.stjude.org/, Human hg19). B) CYFIP2 exon 26-EBF1 exon 7 Sanger sequencing chromatogram (sample 55). C) Chromosome 5q33.3 copy number state, at CYFIP2 and EBF1 locus. Red rectangle represents EBF1 heterozygous deletion. Modified from Chromosome Analysis Suits software (Thermo Fisher Scientific) output figure.

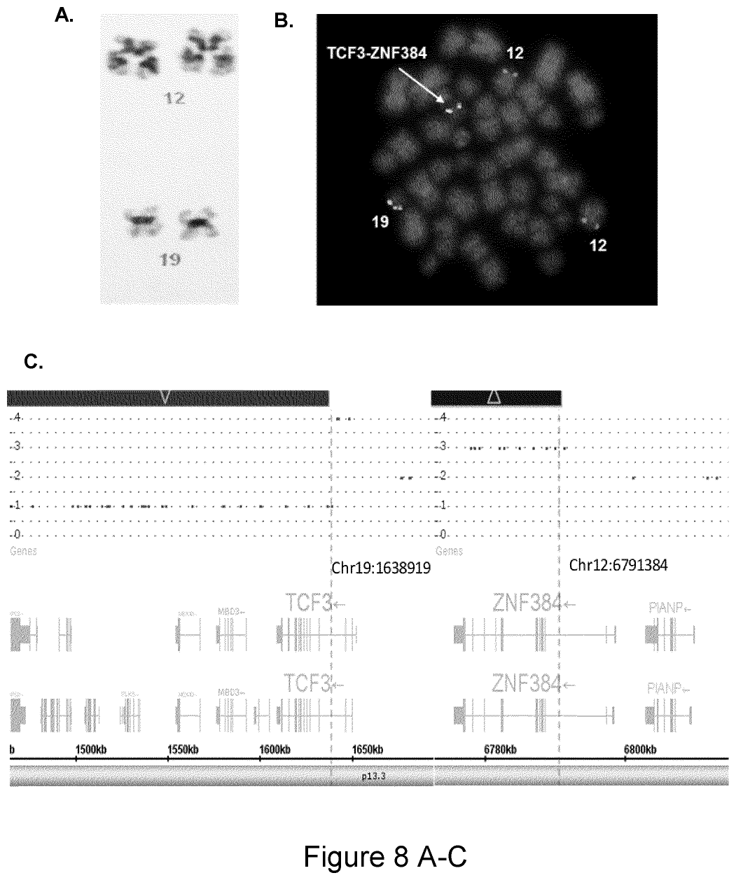

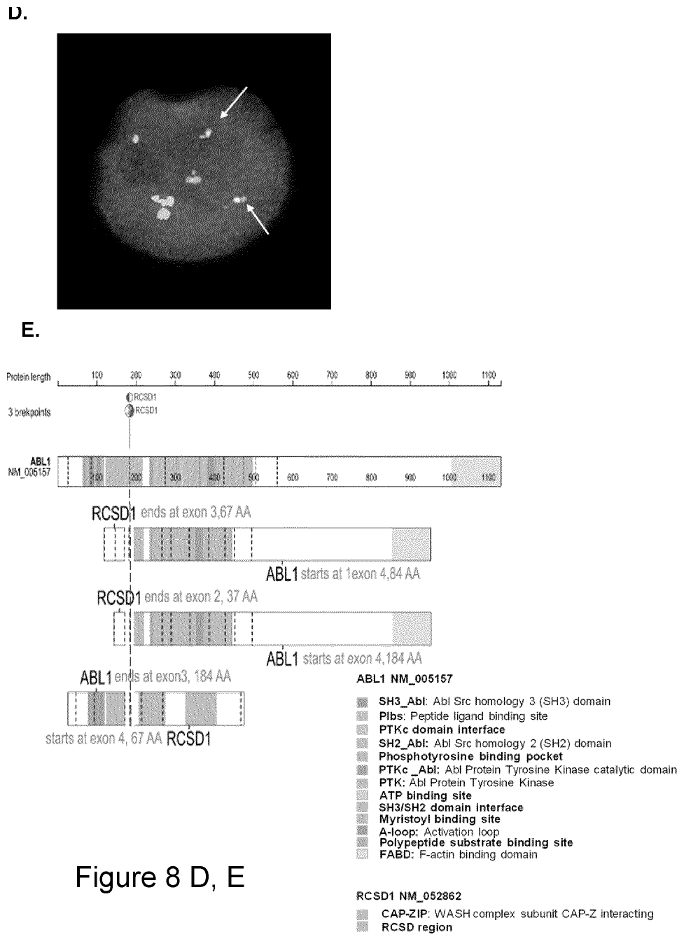

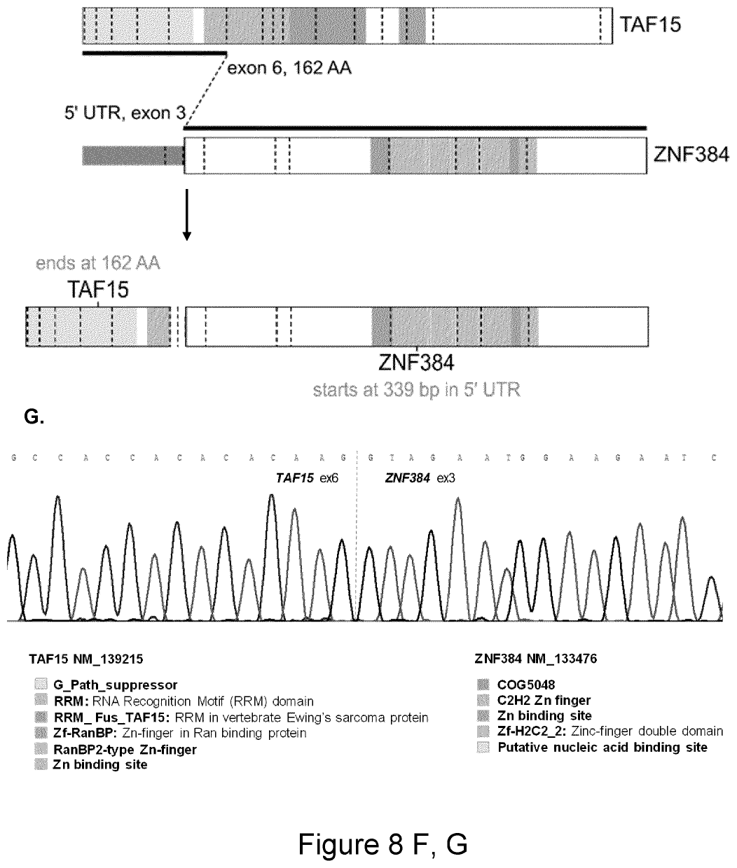

FIG. 8. Identification and validation of B-ALL associated fusion transcripts. A) Partial karyotype of patient 11 showing two normal chromosomes 12 and 19 by CBA. B) FISH performed on metaphase with RP11-433J6 marked in orange, spanning the breakpoint on ZNF384 gene, and RP11-81M8 marked in green, spanning the breakpoint on TCF3 gene, shows one fusion signal on derivative chromosome 19 indicating TCF3-ZNF384 rearrangement. The loss of one expected green signal suggests a partial deletion of TCF3. The arrow indicates the fusion signal. C) Copy Number State of the case 11 at TCF3 and ZNF384 loci (modified from Chromosome Analysis Suits software output figure). Red and blue rectangles represent heterozygous deletion and amplification respectively. D) FISH performed on interphase nucleus of case 22 (pt #13) with RP11-138P14 marked in green, spanning the breakpoint on RCSD1, and Vysis BCR/ABL1/ASS1 Tri-Color DF FISH Probe with ABL1 marked in orange and ASS1 in orange/aqua. One orange/green fusion signal shows RCSD1-ABL1 rearrangement and one orange/aqua/green shows ABL1-RCSD1 rearrangement. The arrows indicate the fusion signals. E) Schematic representation of reciprocal fusions between RCSD1 and ABL1 genes at the same breakpoint (https://proteinpaint.stjude.org/, Human hg19). F) Schematic representation of in frame TAF15-ZNF384 fusion. TAF15 and ZNF384 protein diagrams, domain annotations and in frame fusion scheme between TAF15 exon 26 and ZNF384 exon 12 (TAF15, NM_139215, chr17:34149837, +; ZNF384, NM_133476, chr12: 6788691, −; https://proteinpaint.stjude.org/, Human hg19; sample 21). G) TAF15-ZNF384 in frame fusion breakpoint. Sanger sequencing chromatogram showing the junction of TAF15 exon 6 end and ZNF384 exon 3 start (sample 21).

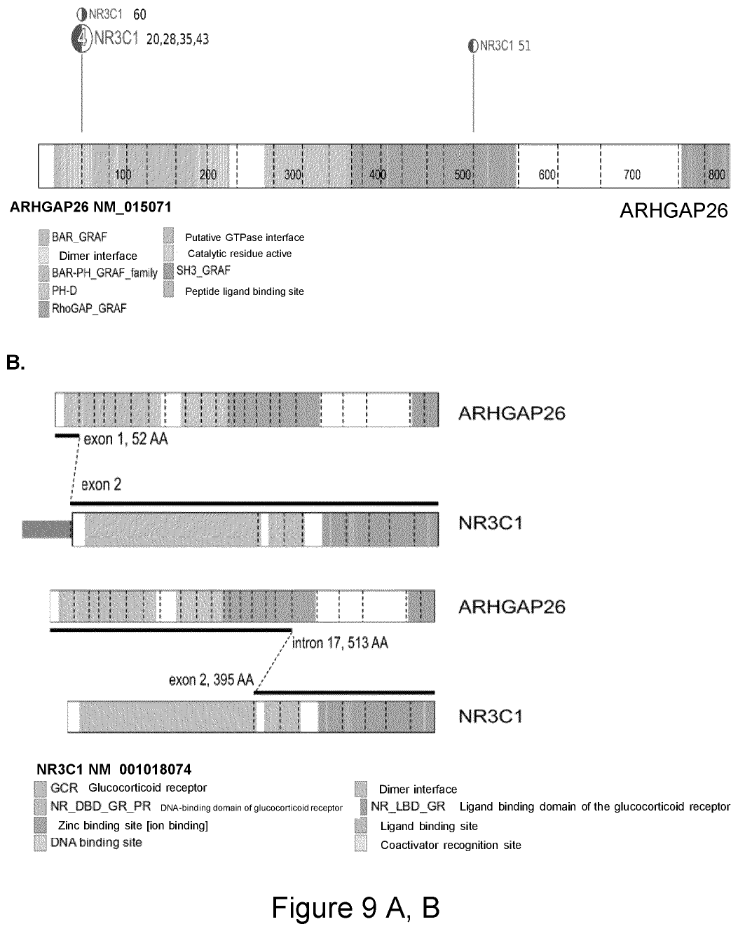

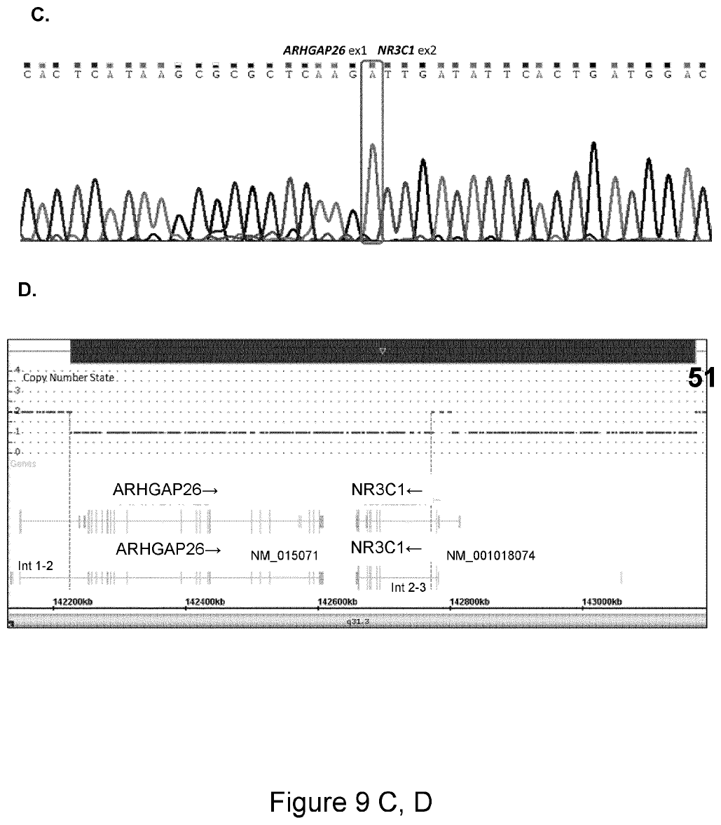

FIG. 9. ARHGAP26-NR3C1 fusions: A) ARHGAP26 protein diagram, domain annotations (https://proteinpaint.stjude.org/, NM_015071, Human hg19) and Ph−/−/− B-ALL cohort breakpoints (samples 20, 28, 35, 43, 51 and the reciprocal fusion NR3C1-ARHGAP26 in sample 60). B) on the left there is represented sample 20, 28, 35 and 43 fusion scheme between ARHGAP26 exon 2 and NR3C1 exon 2 (ARHGAP26, NM_015071, chr5:142150480, +; NR3C1, NM_001018074, chr5: 142780417, −); on the right side there is sample 51 fusion between ARHGAP26 intron 17-18 and NR3C1 exon 3 (ARHGAP26, NM_015071, chr5:142447291, +; NR3C1, NM_001018074, chr5: 142779220, −). C) Example of ARHGAP26 exon1-NR3C1 exon 2 Sanger sequencing chromatogram (sample 20). “A” in the box is shared at the end of ARHGAP26 exon1 and at the beginning of NR3C1 exon 2, in all samples with this breakpoint (20,28,35 and 43). D) sample 51 chromosome 5q31.3 copy number state, at ARHGAP26 and NR3C1 locus. Red rectangle represents heterozygous deletion. Modified from Chromosome Analysis Suits software (Thermo Fisher Scientific) output figures.

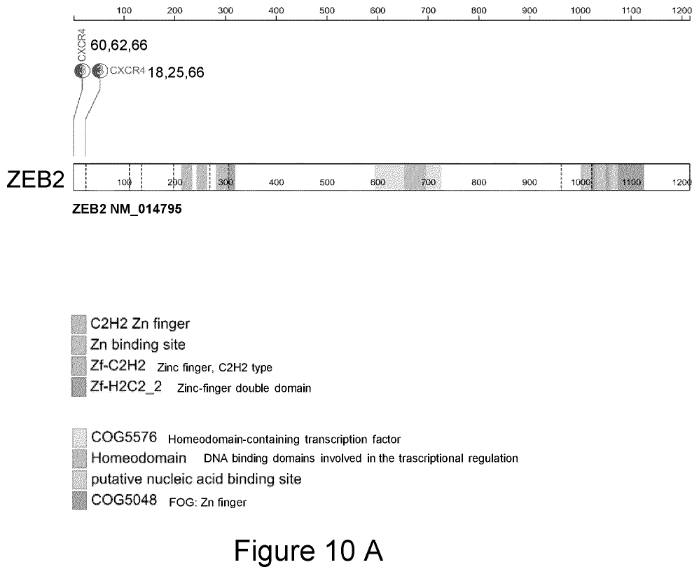

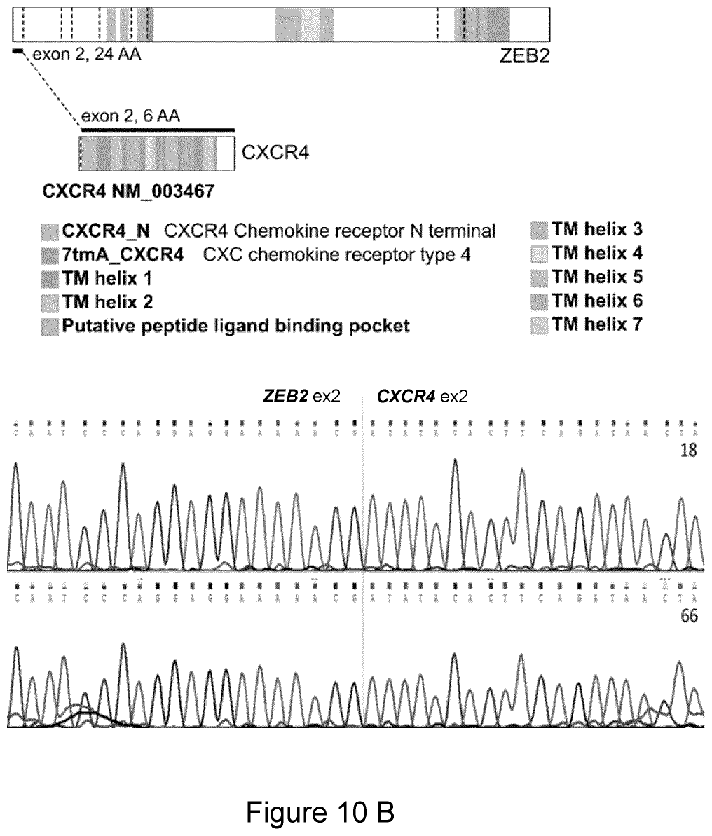

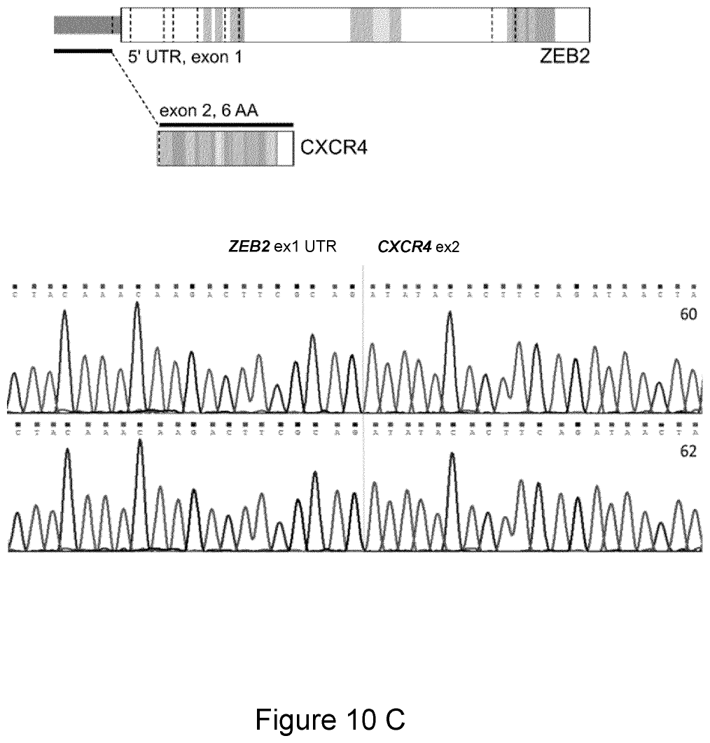

FIG. 10. ZEB2-CXCR4 fusions: A) ZEB2 protein diagram, domain annotations (https://proteinpaint.stjude.org/, NM_014795, Human hg19) and Ph−/−/− B-ALL cohort two breakpoints (samples 18,25,60, 62 and 66). B) on the left there is represented sample 18, 25 and 66 in frame fusion scheme between ZEB2 exon 2 and CXCR4 exon 2 (ZEB2, NM_014795, chr2: 145274845, −; CXCR4, NM_003467, chr2: 136873482, −); on the right side there are ZEB2 exon 2-CXCR4 exon 2 Sanger sequencing chromatogram (samples 18 and 66). C) on the left there is represented sample 60, 62 and 66 in frame fusion scheme between ZEB2 exon 1 UTR and CXCR4 exon 2 (ZEB2, NM_014795, chr2: 145277506, −; CXCR4, NM_003467, chr2: 136873482, −); on the right side there are ZEB2 exon 1 UTR-CXCR4 exon 2 Sanger sequencing chromatogram (samples 60 and 62).

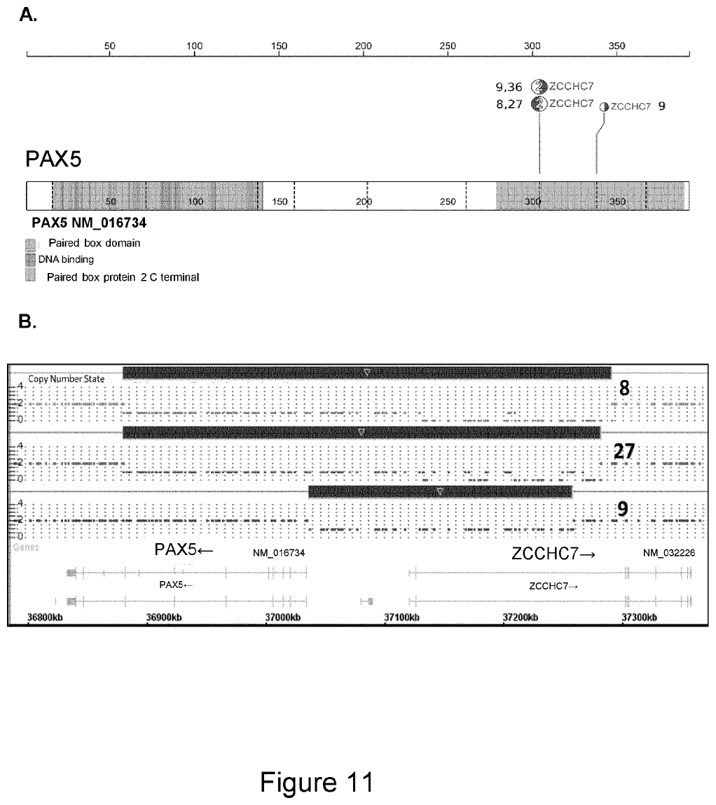

FIG. 11. PAX5-ZCCHC7 fusions: A) PAX5 protein diagram, domain annotations (https://proteinpaint.stjude.org/, NM_016734, Human hg19) and Ph−/−/− B-ALL cohort breakpoints (samples 8,9,27 and 36). B) chromosome 9p13.2 copy number state, at PAX5 and ZCCHC7 locus, of pt #1 at diagnosis (sample 8) and relapse (sample 27) and on pt #2 (sample 9). Red rectangle represents heterozygous deletion. Modified from Chromosome Analysis Suits software (Thermo Fisher Scientific) output figures.

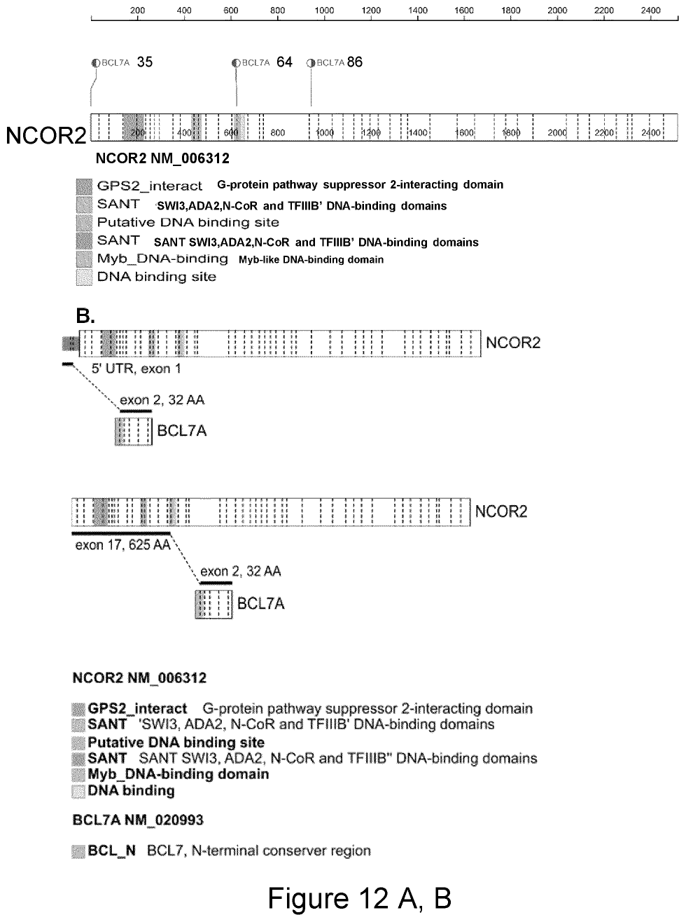

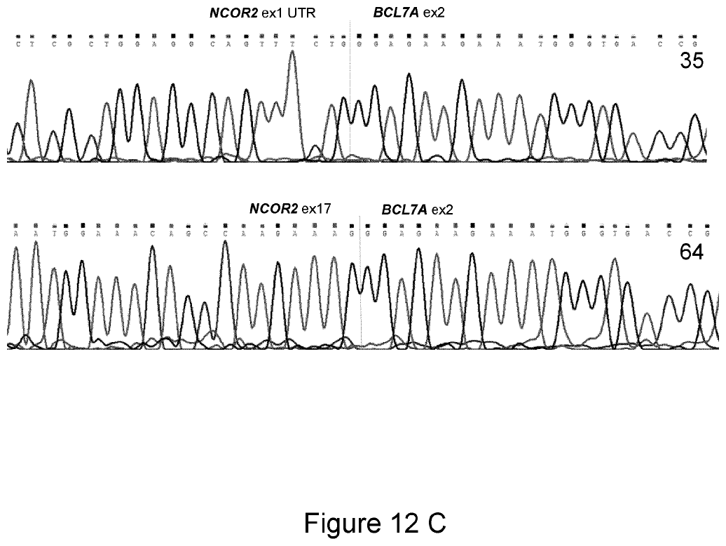

FIG. 12. NCOR2-BCL7A fusions: A) NCOR2 protein diagram, domain annotations (https://proteinpaint.stjude.org/, NM_006312, Human hg19) and Ph−/−/− B-ALL cohort three breakpoints (samples 35, 64 and 86). B) on the left there is represented sample 35 in frame fusion scheme between NCOR2 exon 1 UTR and BCL7A exon 2 (NCOR2, NM_006312, chr12: 125020111, −; BCL7A, NM_020993, chr12: 122468606, +); on the right side there is represented sample 64 in frame fusion scheme between NCOR2 exon 17 and BCL7A exon 2 (NCOR2, NM_006312, chr12: 124882665, −; BCL7A, NM_020993, chr12: 122468606, +); C) NCOR2 exon 1 UTR-BCL7A exon 2 Sanger sequencing chromatogram (samples 35) and NCOR2 exon 17-BCL7A exon 2 Sanger sequencing chromatogram (samples 64).

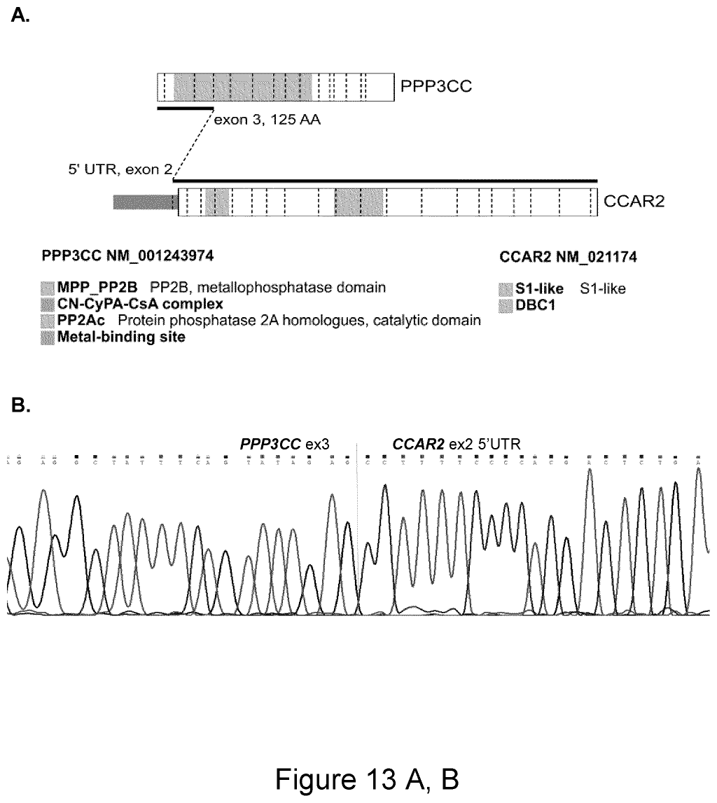

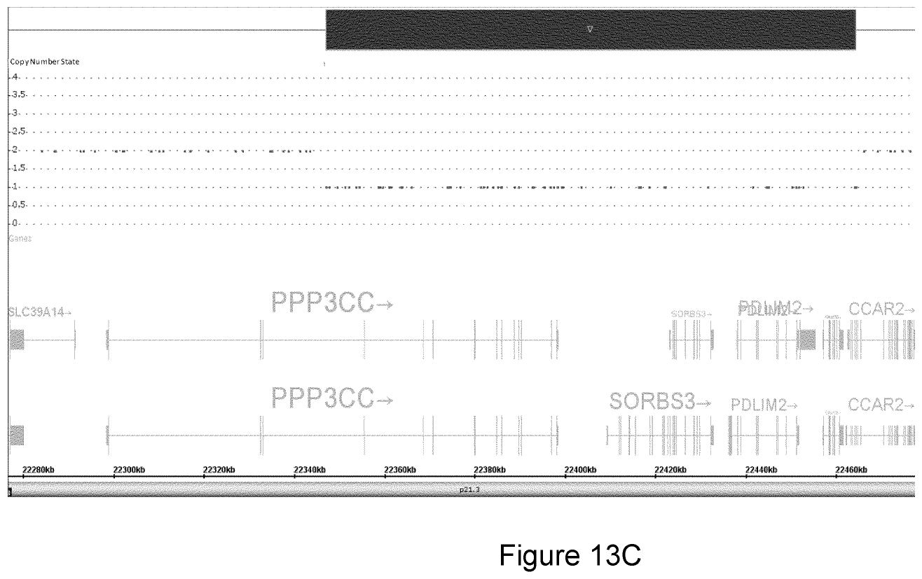

FIG. 13. PPP3CC-CCAR2 fusion: A) PPP3CC and CCAR2 protein diagrams, domain annotations and in frame fusion scheme between PPP3CC exon 3 and CCAR2 exon 2 (PPP3CC, NM_001243974, chr8: 22333137, +; CCAR2: NM_021174, chr8: 22463249, +; https://proteinpaint.stjude.org/, Human hg19). B) PPP3CC exon 3-CCAR2 exon 2 Sanger sequencing chromatogram (sample 46). C) chromosome 8q21.3 copy number state, at PPP3CC and CCAR2 locus. Red rectangle represents heterozygous deletion. Modified from Chromosome Analysis Suits software (Thermo Fisher Scientific) output figure.

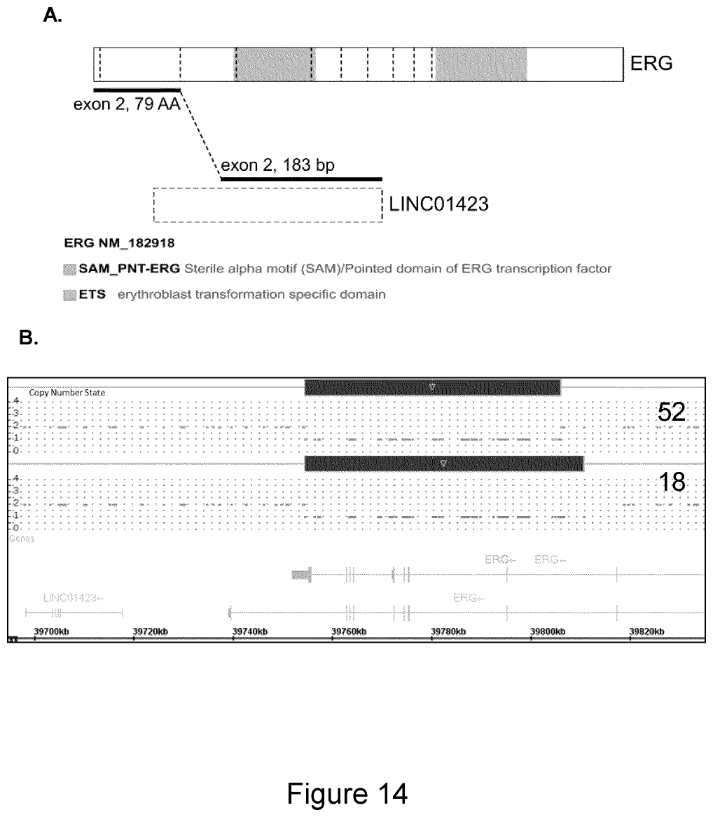

FIG. 14. ERG-LINC01423 fusions: A) ERG and LINC01423 protein diagrams, domain annotations and in frame fusion scheme between ERG exon 2 and LINC01423 exon 2 (ERG, NM_182918, chr2l: 39817327, −; LINC01423, NR_110545, chr2l: 39705298, −; https://proteinpaint.stjude.org/, Human hg19). B) Chromosome 21q22.2 copy number state, at ERG and LINC01423 locus, of pt #10 at diagnosis (sample 18) and relapse (sample 52). Red rectangle represents heterozygous ERG deletion. Modified from Chromosome Analysis Suits software (Thermo Fisher Scientific) output figures.

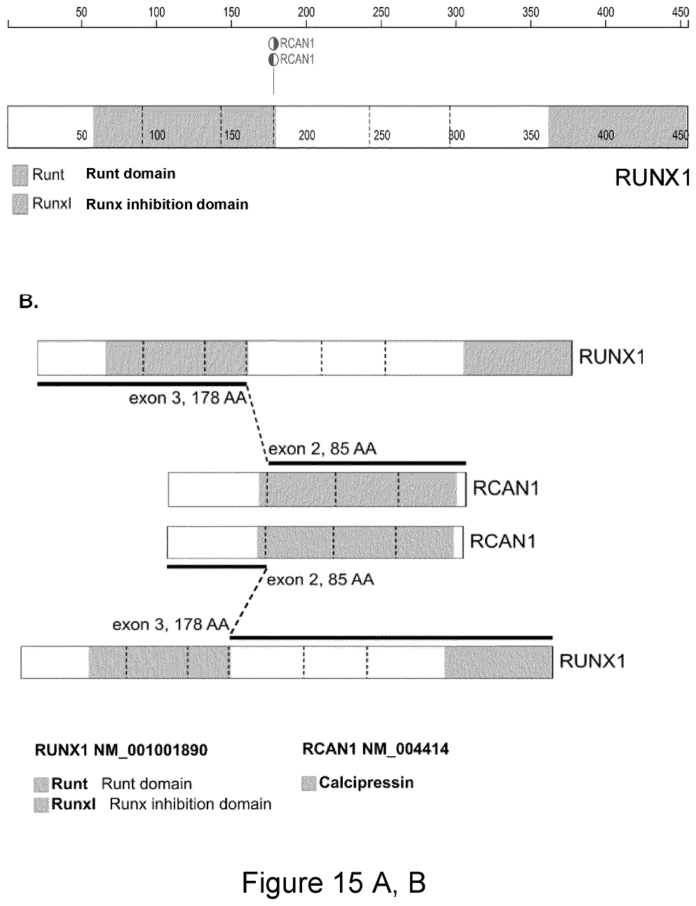

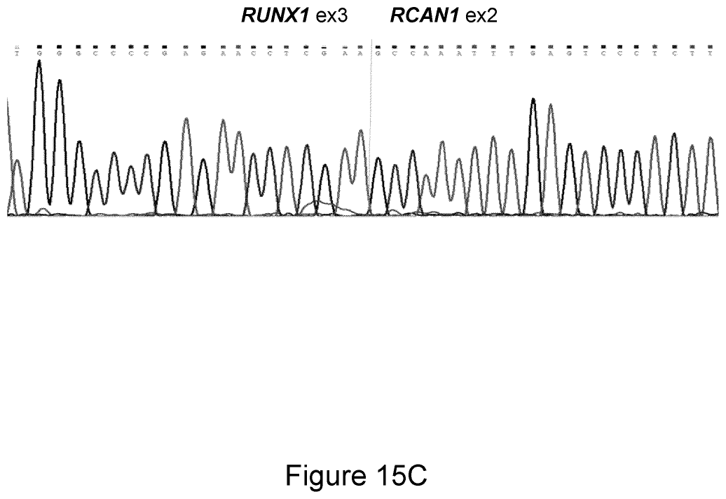

FIG. 15. RUNX1-RCAN1 fusions: A) RUNX1 protein diagram, domain annotations (https://proteinpaint.stjude.org/, NM_001001890, Human hg19) and the reciprocal breakpoint (samples 20). B) on the left is represented in frame fusion schemes between RUNX1 exon 3 and RCAN1 exon 2 (RUNX1, NM_001001890, chr2l: 36231771, −; RCAN1, NM_004414, chr2l: 35896008, −); on the right side in frame fusion schemes between RCAN1 exon 2 and RUNX1 exon 3 (RCAN1, NM_004414, chr2l: 35896007, −; RUNX1, NM_001001890, chr2l: 36231770, −). C) Sanger sequencing chromatogram of RUNX1 exon 3 and RCAN1 exon 2 fusion.

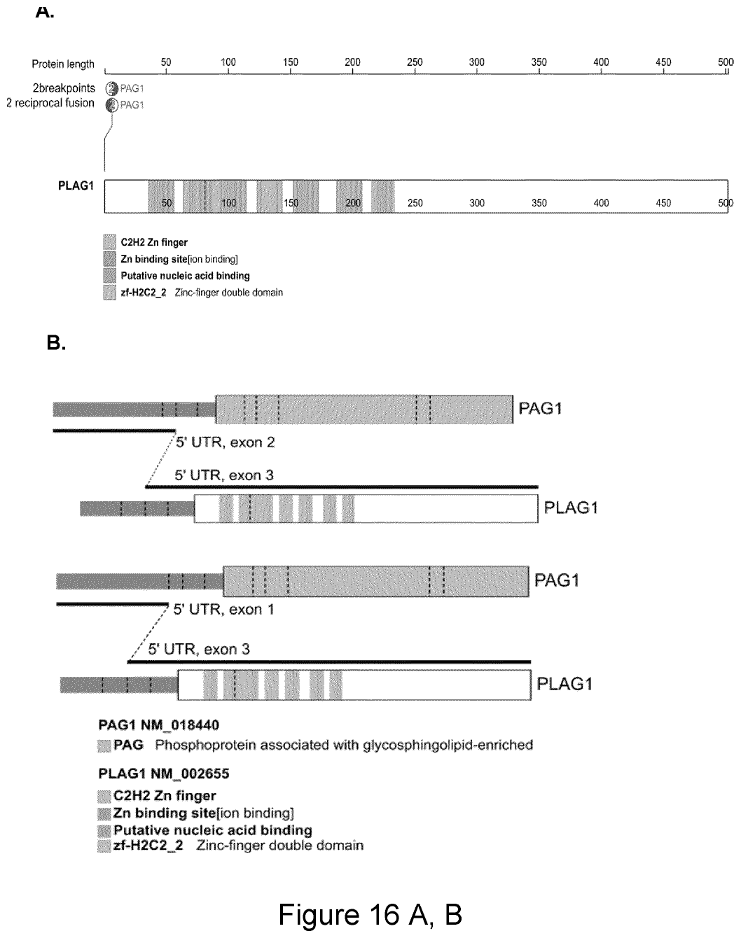

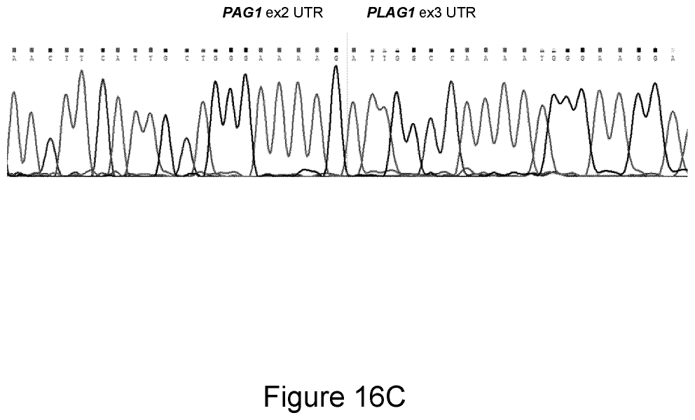

FIG. 16. PAG1-PLAG1 fusions: A) PLAG1 protein diagram, domain annotations (https://proteinpaint.stjude.org/, Human hg19) and the reciprocal breakpoints (sample 51). B) on the left is represented fusion schemes between PAG1 exon 2 UTR and PLAG1 exon 3 UTR (PAG1, NM_018440, chr8: 81982347; −; PLAG1, NM_002655, chr8: 57083748, −); on the right side is represented fusion schemes between PAG1 exon 1 UTR and PLAG1 exon 3 UTR (PAG1, NM_018440, chr8: 81982347; −; PLAG1, NM_002655, chr8: 57083748, −). C) Sanger sequencing chromatogram of PAG1 exon 2 UTR and PLAG1 exon 3 UTR fusion.

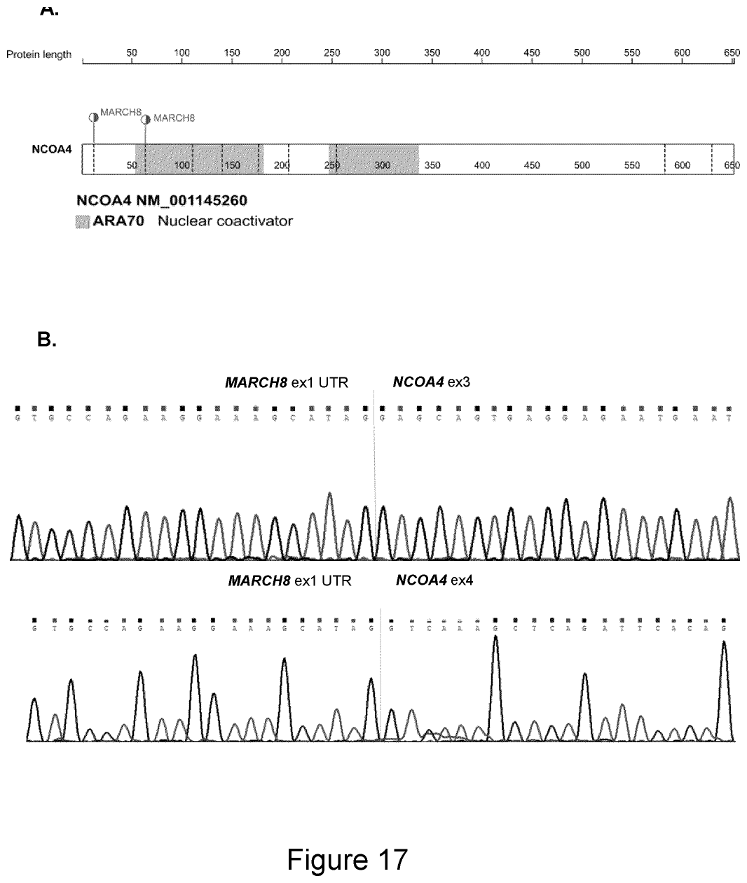

FIG. 17. MARCH8-NCOA4 fusions: A) NCOA4 protein diagram, domain annotations (https://proteinpaint.stjude.org/, NM_001145260, Human hg19) and two breakpoint positions (sample 68). B) MARCH8 exon 1 UTR-NCOA4 exon 3 Sanger sequencing chromatogram (MARCH8, NM_145021, chr10: 46089683, −; NCOA4, NM_001145260, chr10: 51579126, −) and MARCH8 exon 1 UTR-NCOA4 exon 4 Sanger sequencing chromatogram (MARCH8, NM_145021, chr10: 46089683, −; NCOA4, NM_001145260, chr10: 51580555, −).

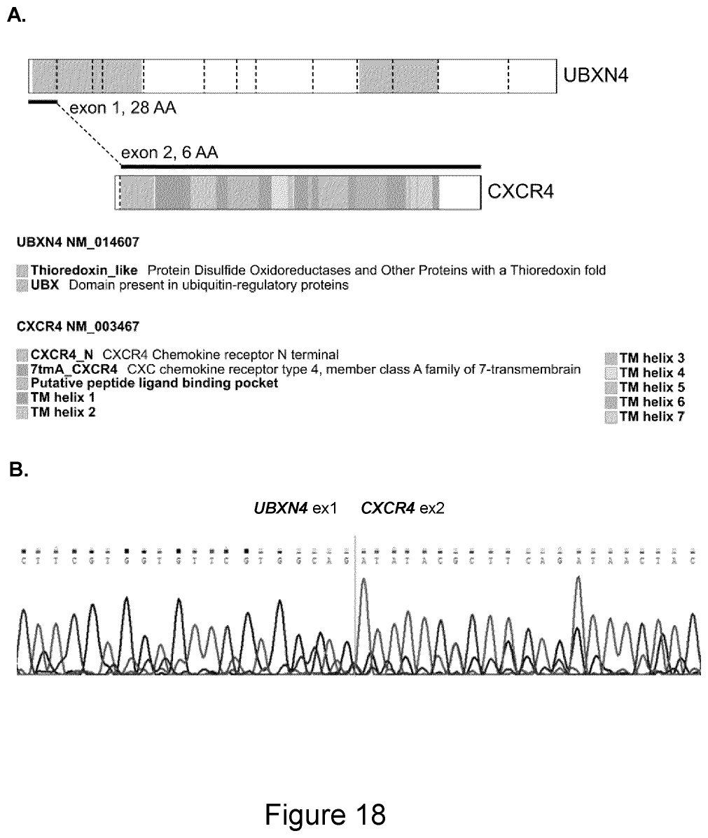

FIG. 18. UBXN4-CXCR4 fusion: A) UBXN4 and CXCR4 protein diagrams, domain annotations and in frame fusion scheme between UBXN4 exon 1 and CXCR4 exon 2 (UBXN4, NM_014607, chr2: 136499581, +; CXCR4, NM_003467, chr2: 136873482, −; https://proteinpaint.stjude.org/, Human hg19). B) UBXN4 exon 1-CXCR4 exon 2 fusion Sanger sequencing chromatogram (samples 28).

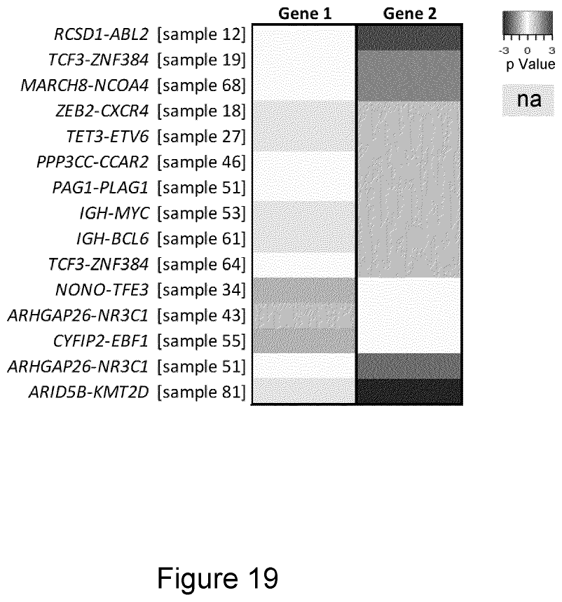

FIG. 19: Significant differential expression evaluation of both fusion gene partners in Ph−/−/− patient samples with the fusion compared with samples not harboring any fusion involving that genes. Blue and red chromatic scale rectangles represent gene 1 and gene 2 significant overexpression (>0), downregulation (<0) or equal expression (=0; white) between with or without the fusion. Not available (na) panel data are represented with grey rectangles. Heatmap was performed using online tool Heatmapper[47].

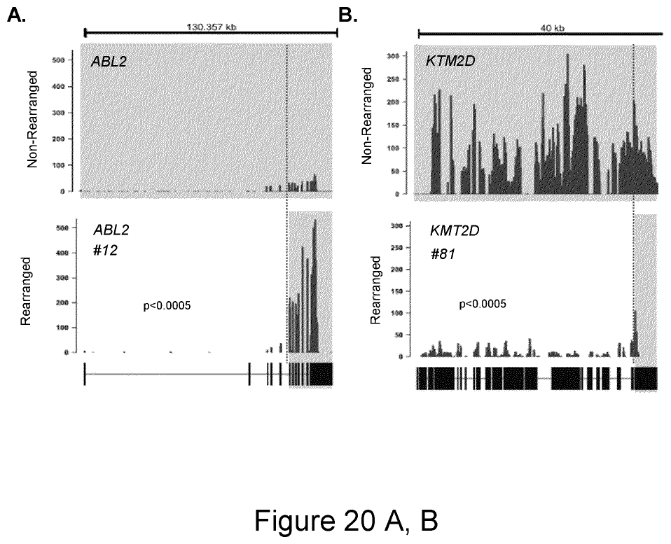

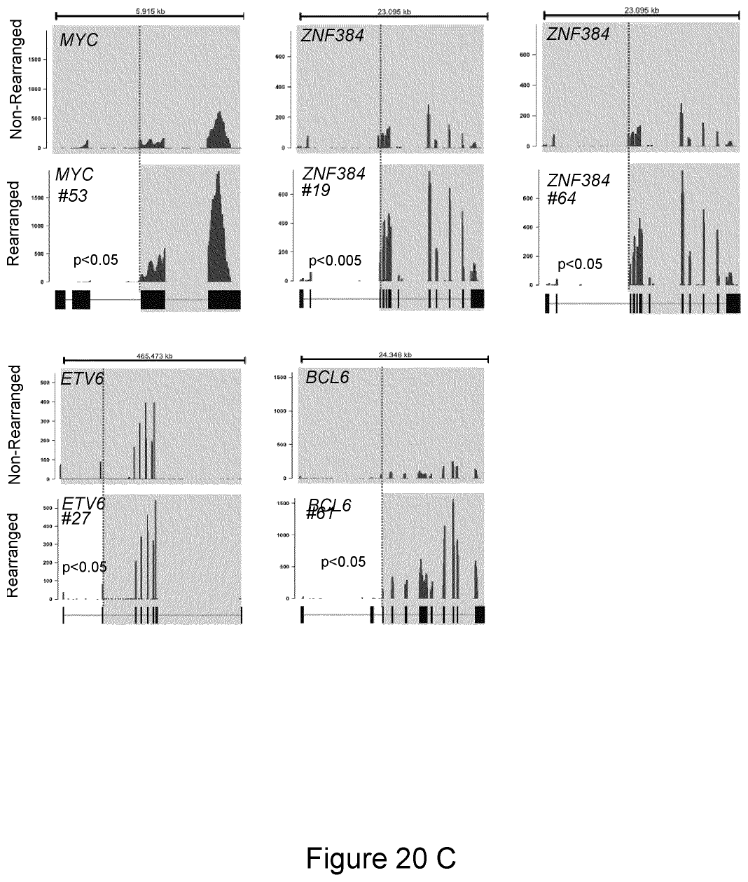

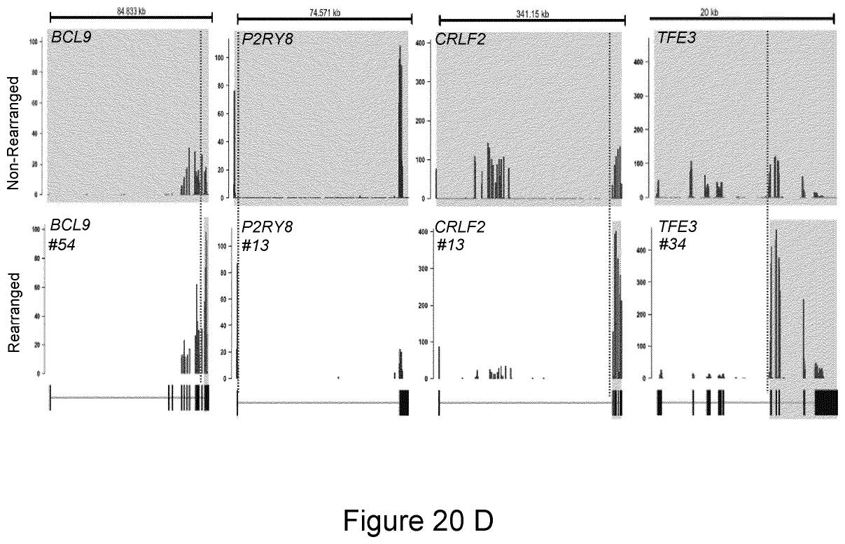

FIG. 20. Gene partner average read depth fusion plot comparisons between fused sample and samples without two gene any fusions: A) ABL2 of RCSD1-ABL2 fusion (sample 12). B) KMT2D of ARIDB5-KMT2D (sample 81). C) MYC of IGH-MYC (sample 53); ZNF384 of TCF3-ZNF384 (sample 19 and 64); ETV6 of TET3-ETV6 (sample 27) and BCL6 of IGH-BCL6 (sample 61). D) BCL9 of MEF2D-BCL9 (sample 54); P2RY8-CRLF2 (sample 13) and TFE3 of NONO-TFE3 (sample 34). Grey background represents retained part of fusion gene partners in samples with the fusion (Rearranged) and in all other Ph−/−/− non fused samples (Non-Rearranged).

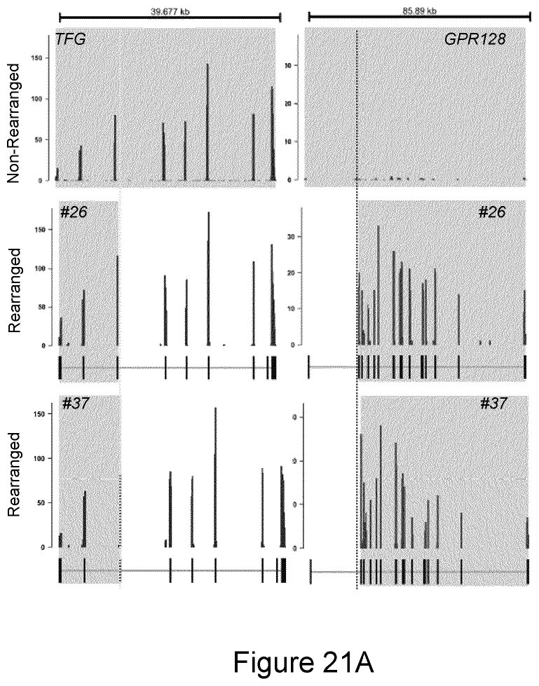

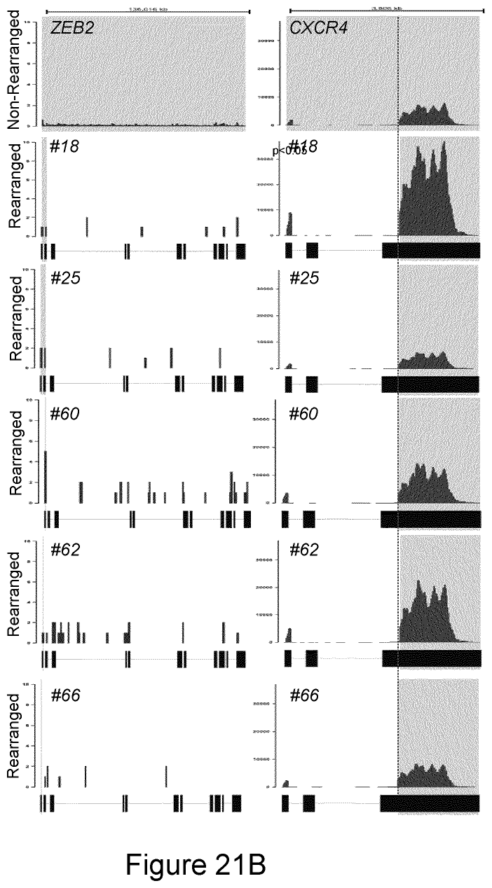

FIG. 21. Fusion gene partner read depth fusion plot comparisons between fused samples and samples without two gene any fusions: A) TFG-GPR128 fusions (samples 26 and 37). B) ZEB2-CXCR4 (samples 18, 25, 60, 62 and 66). Grey background represents retained part of fusion gene partners in samples with the fusion (Rearranged) and in all other Ph−/−/− non fused samples (Non-Rearranged).

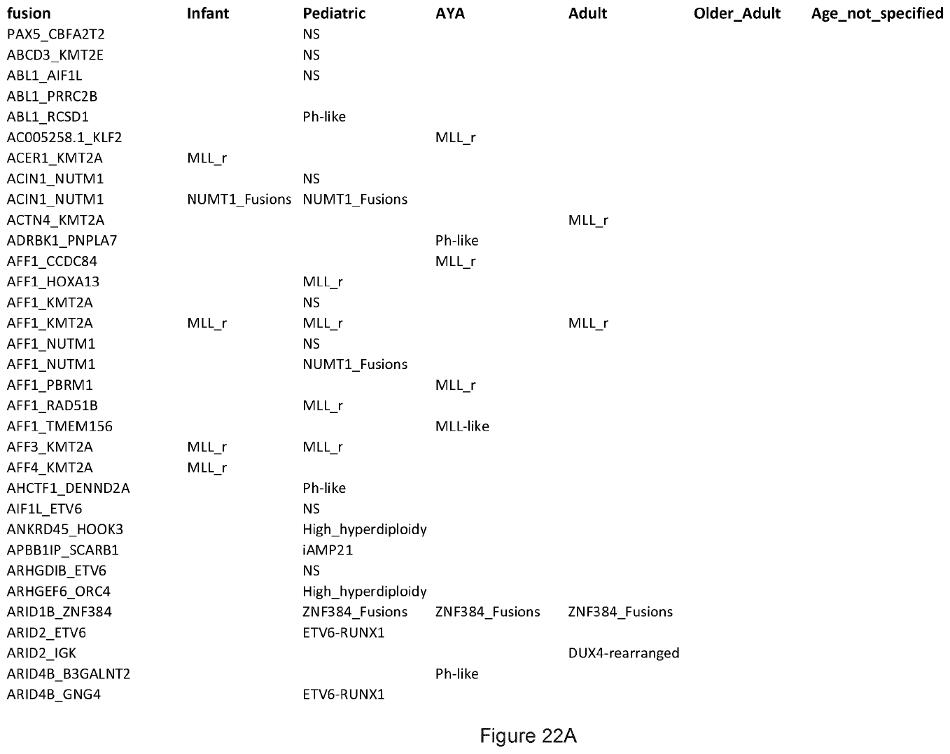

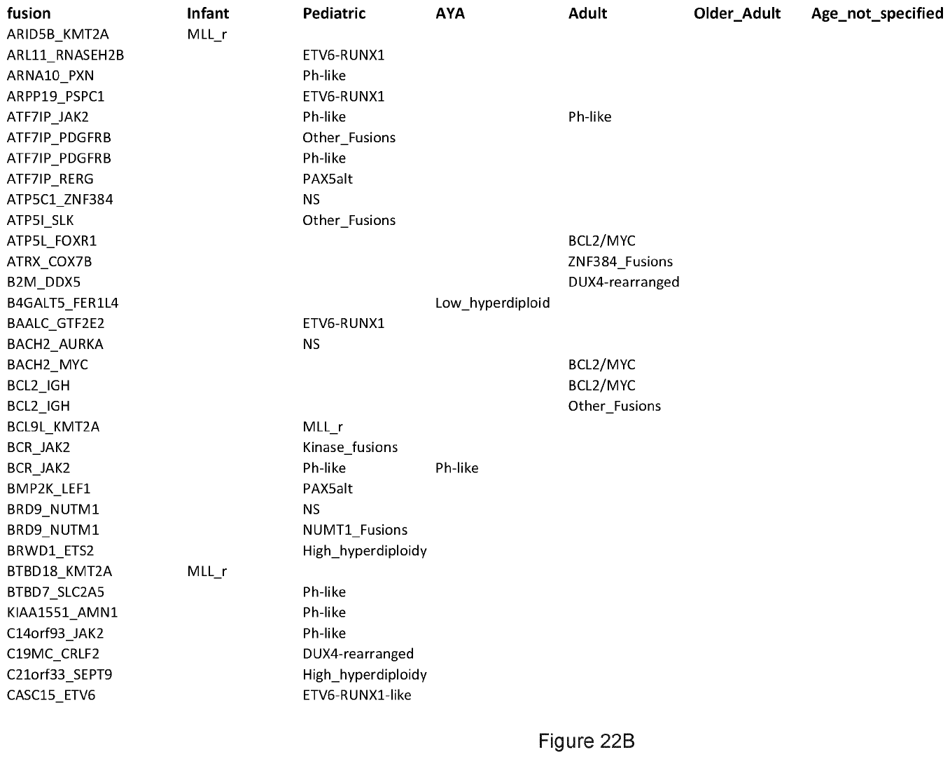

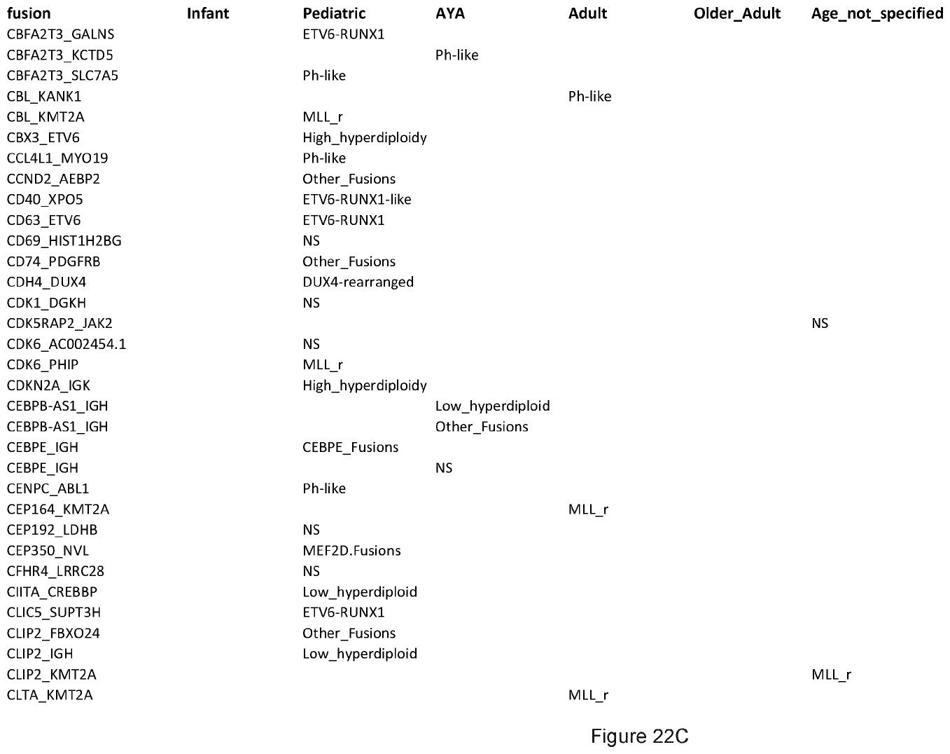

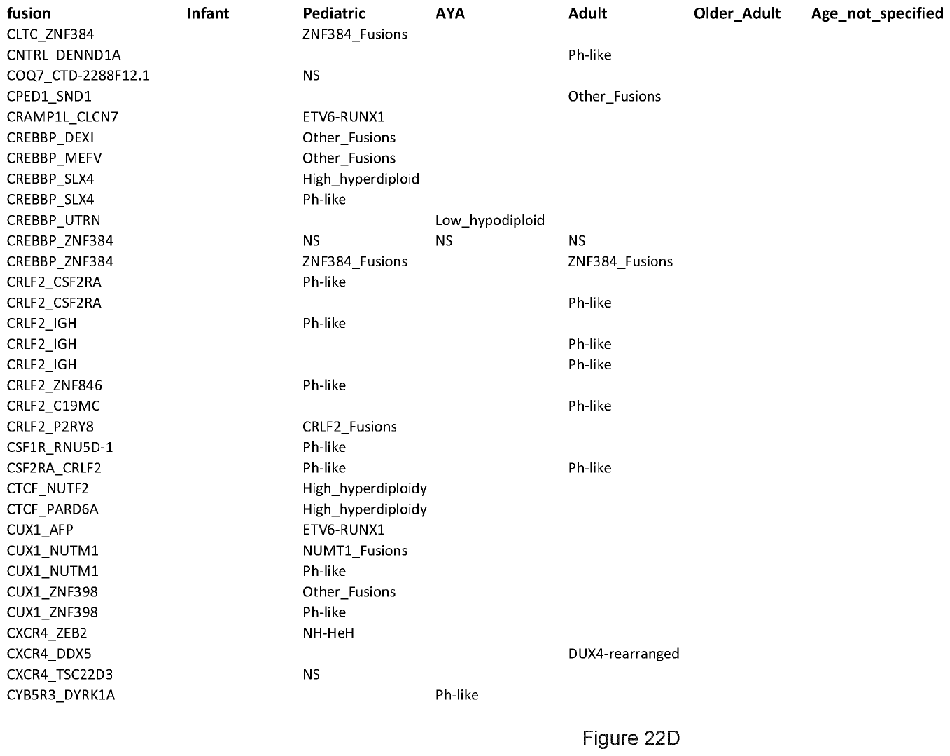

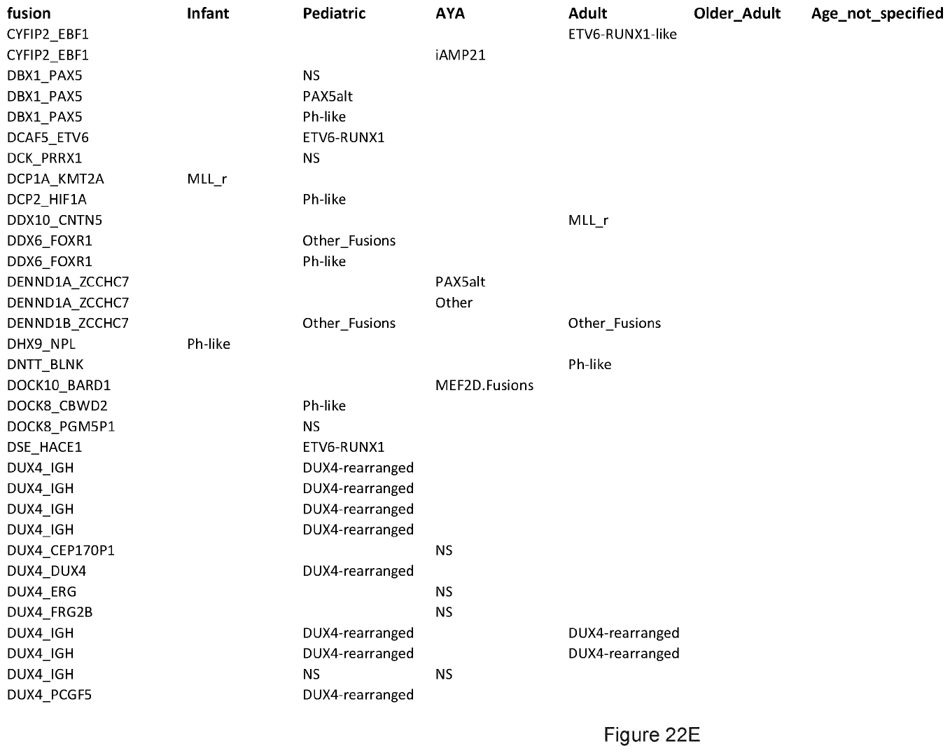

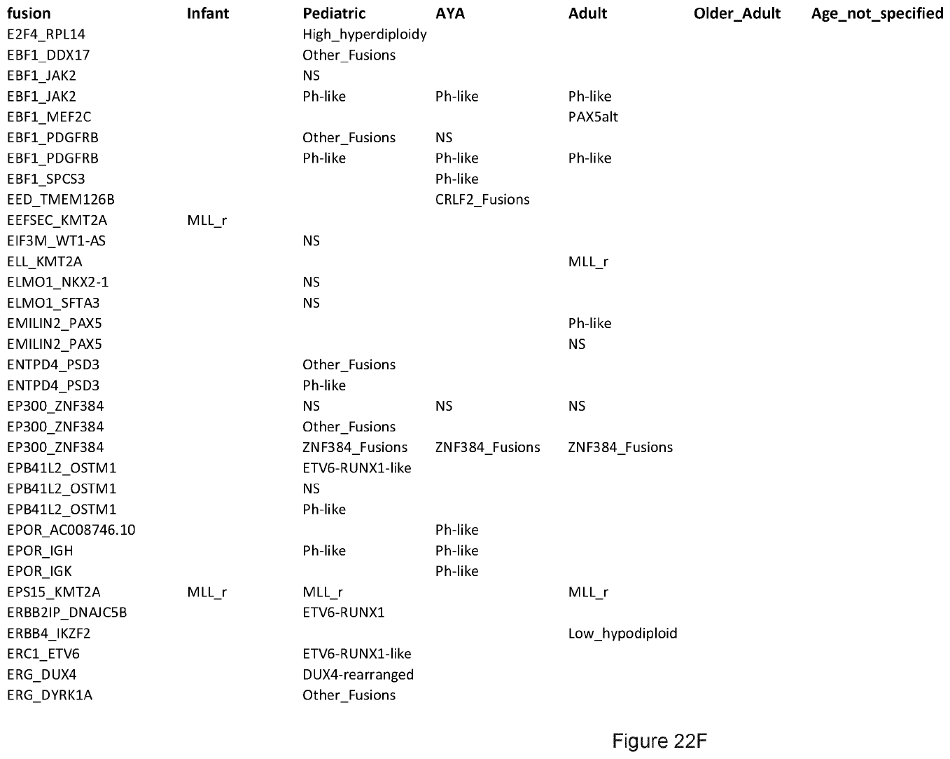

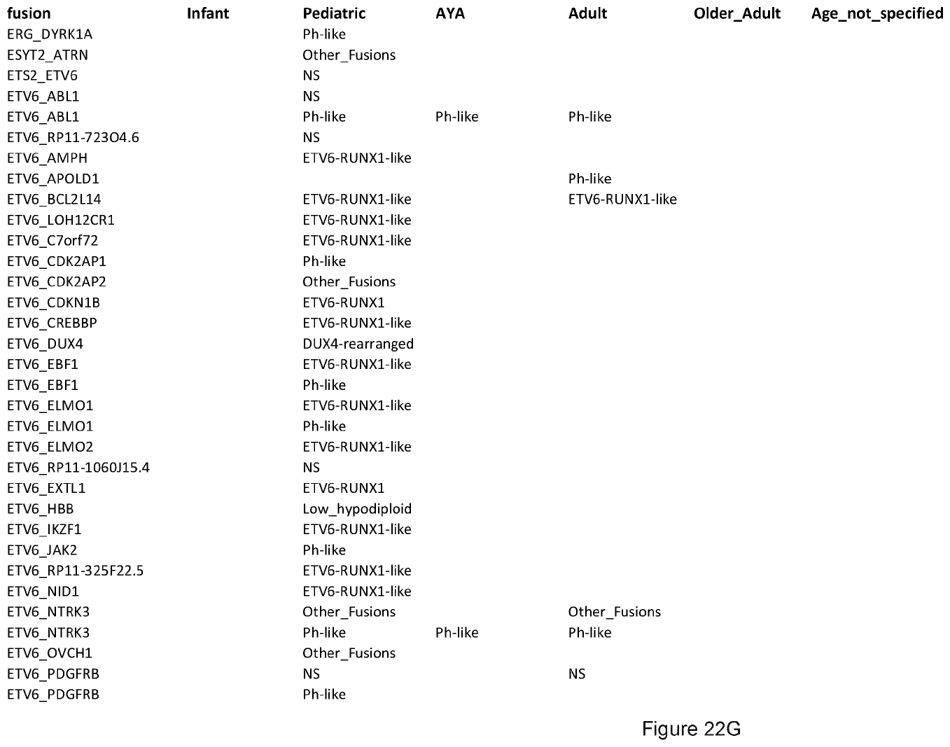

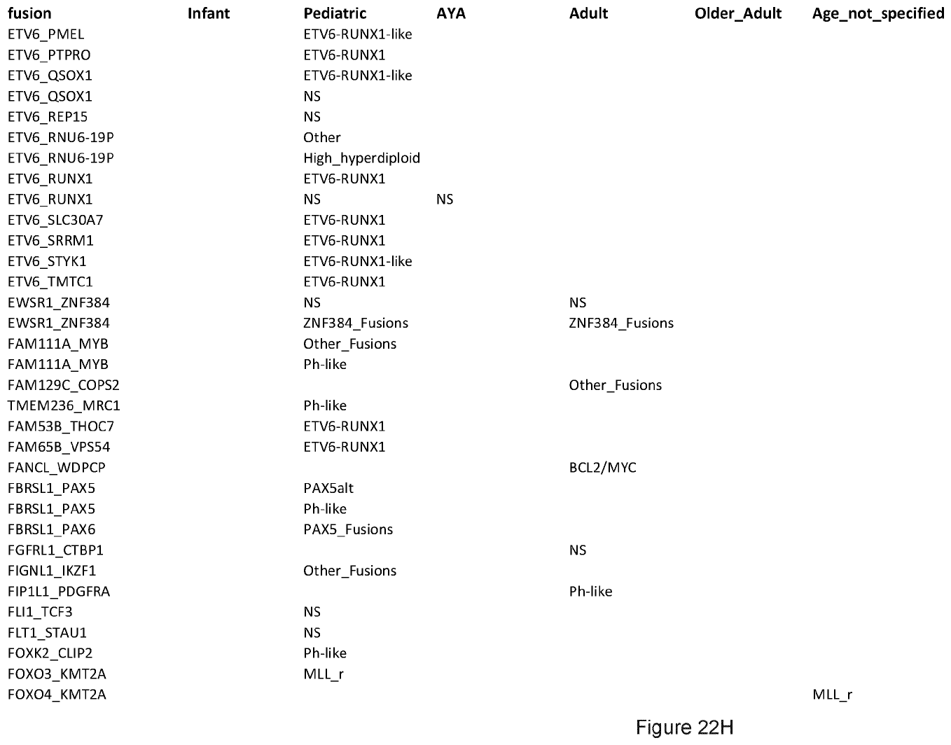

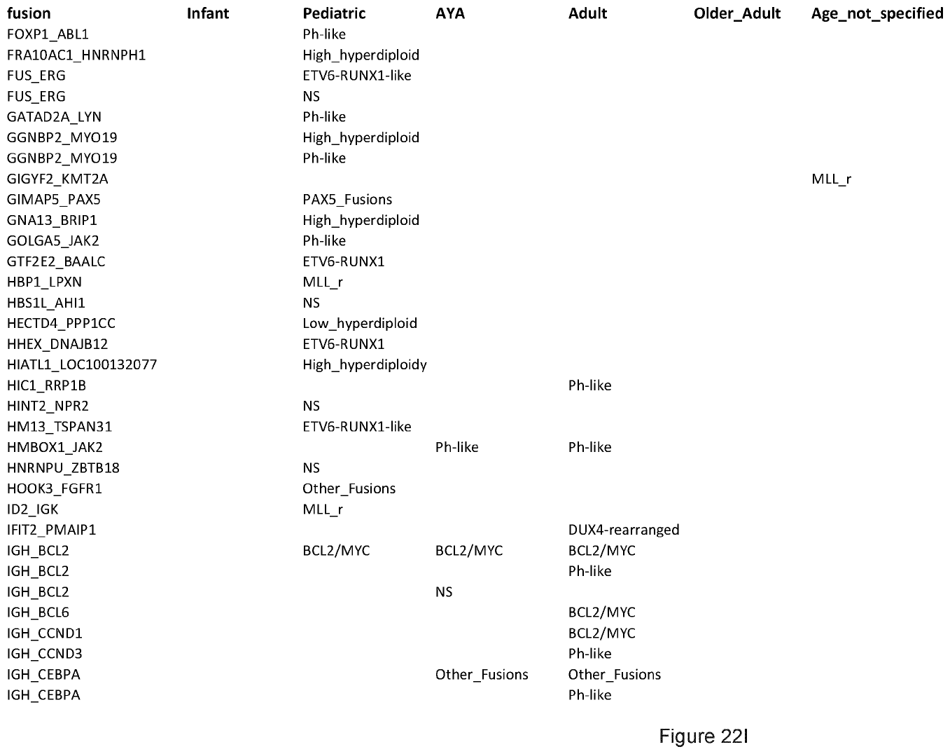

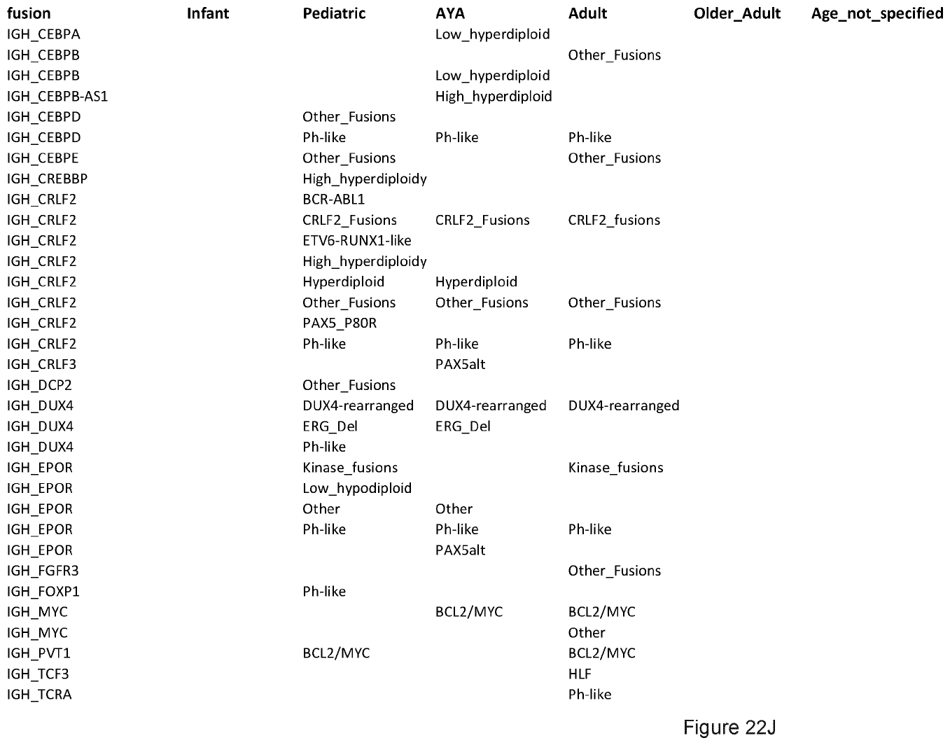

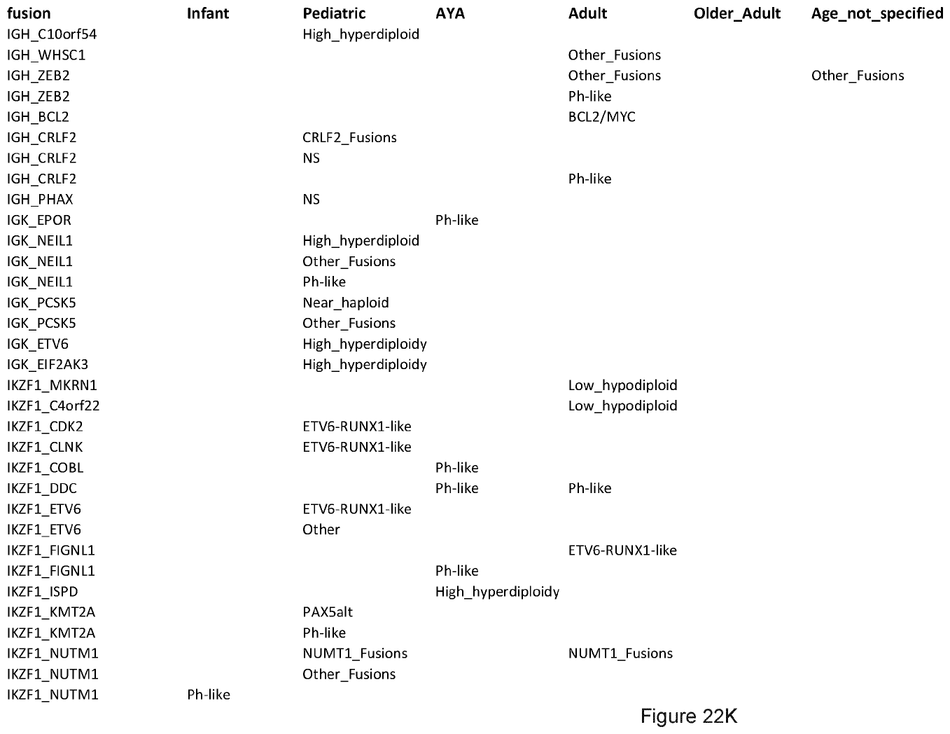

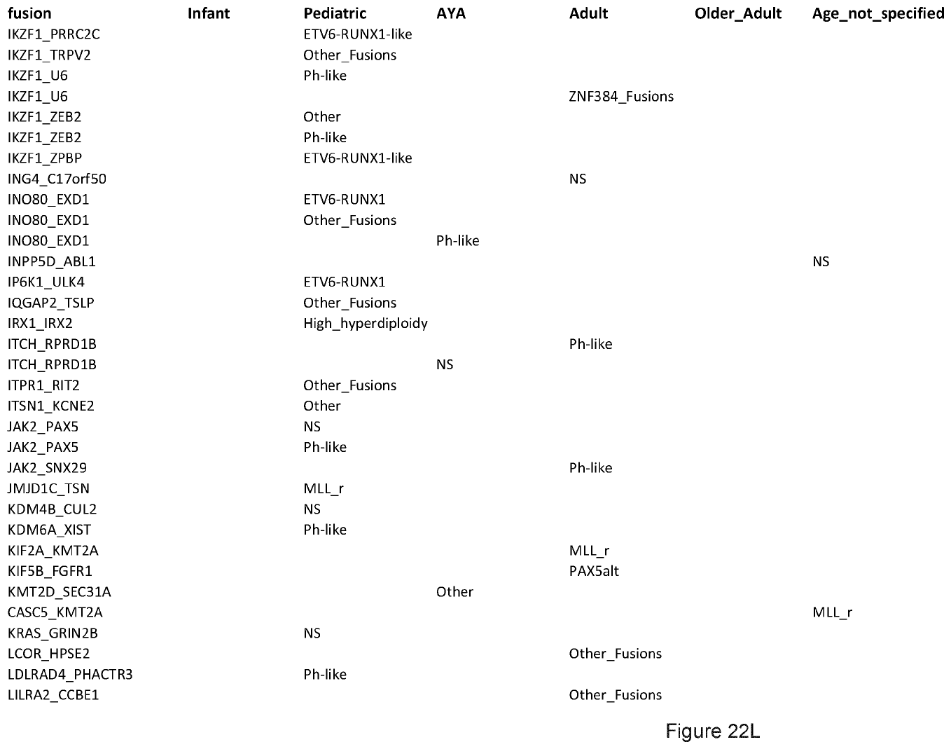

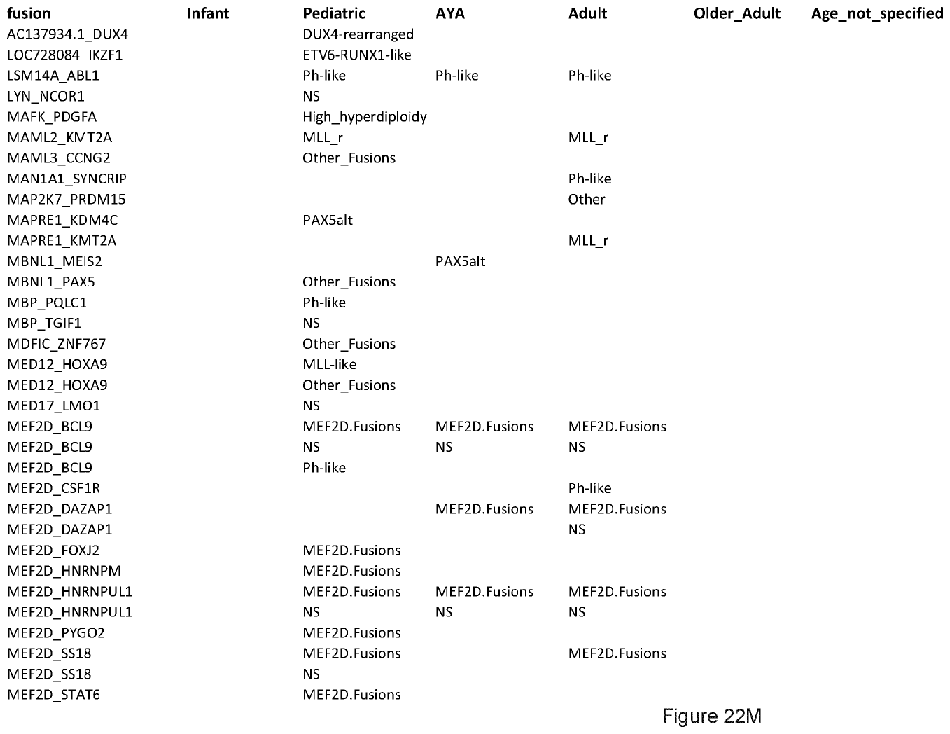

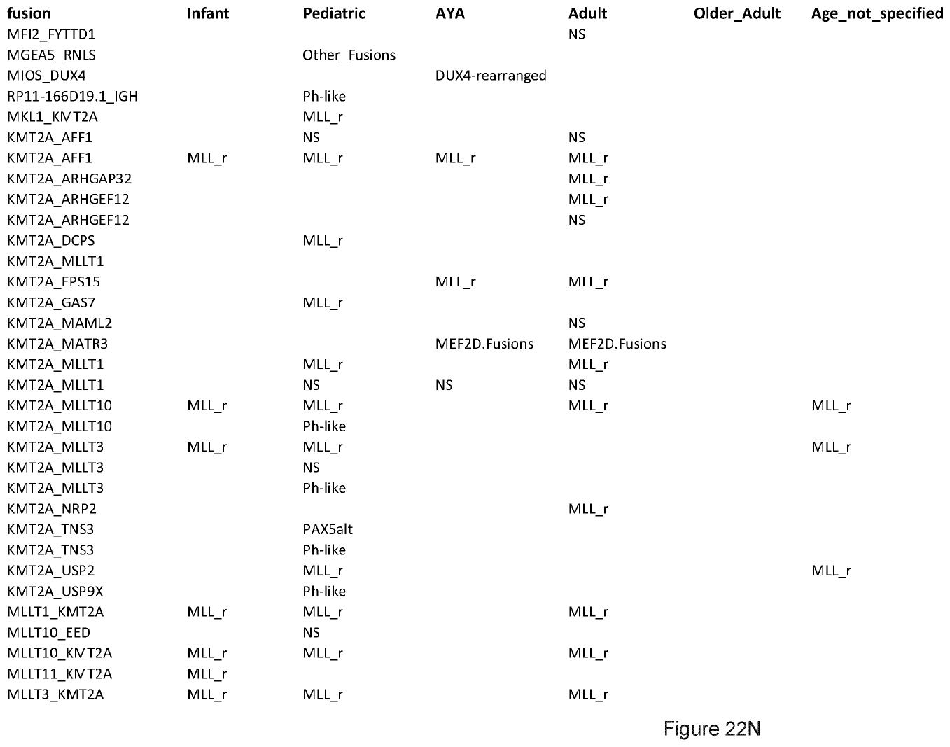

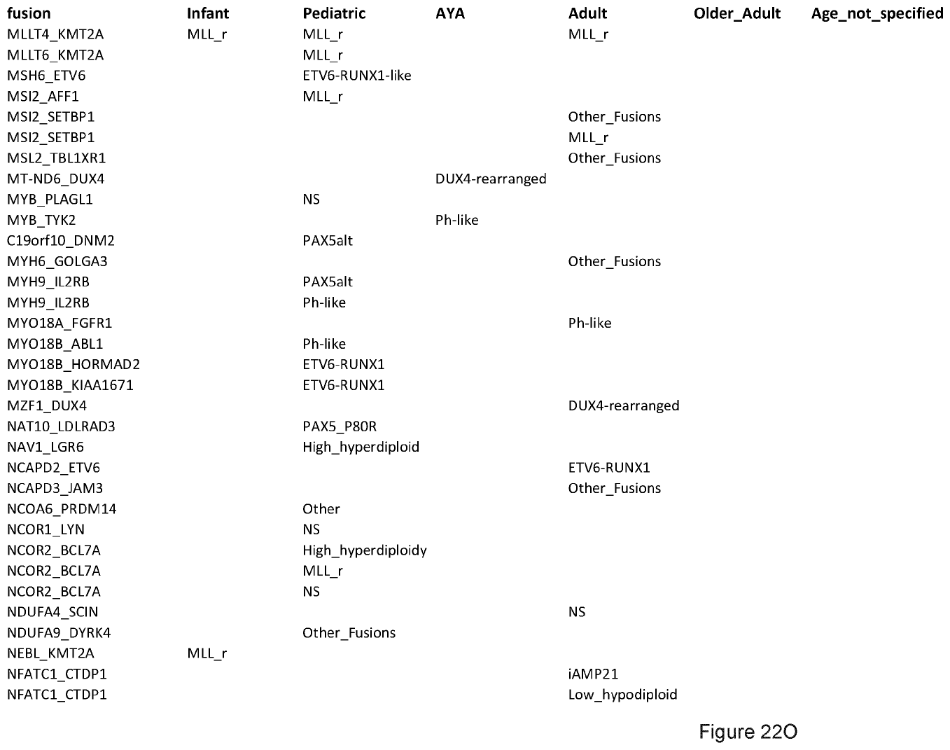

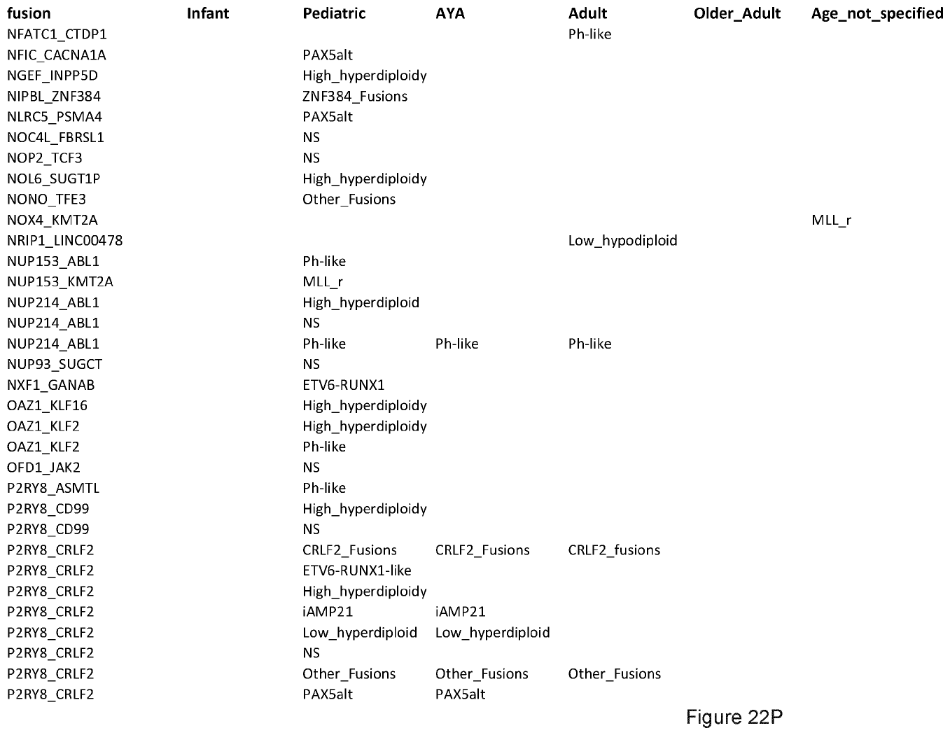

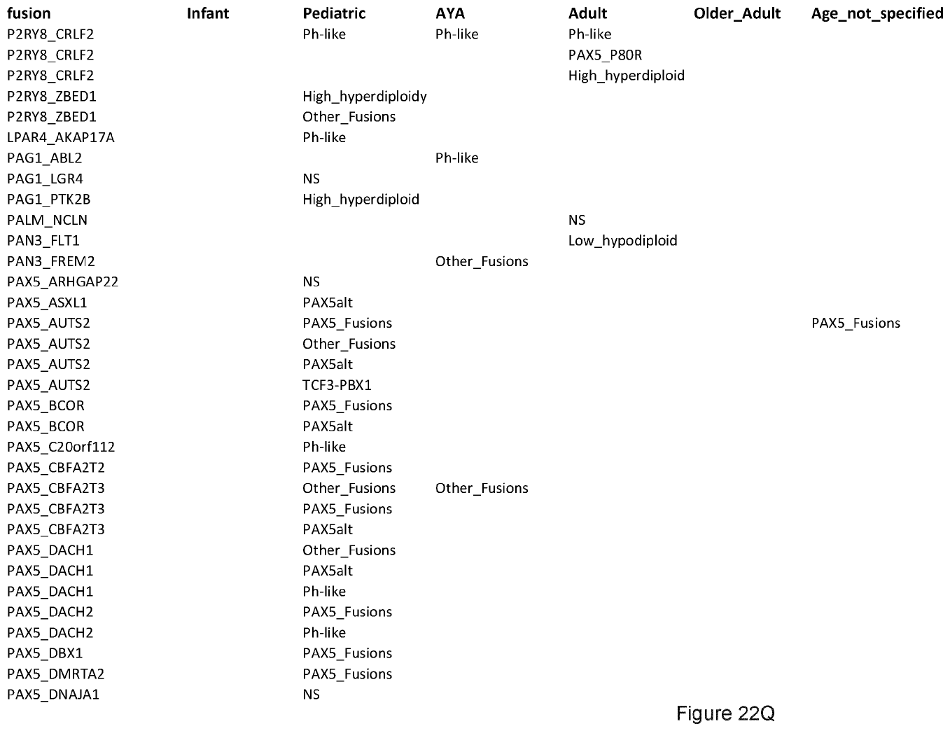

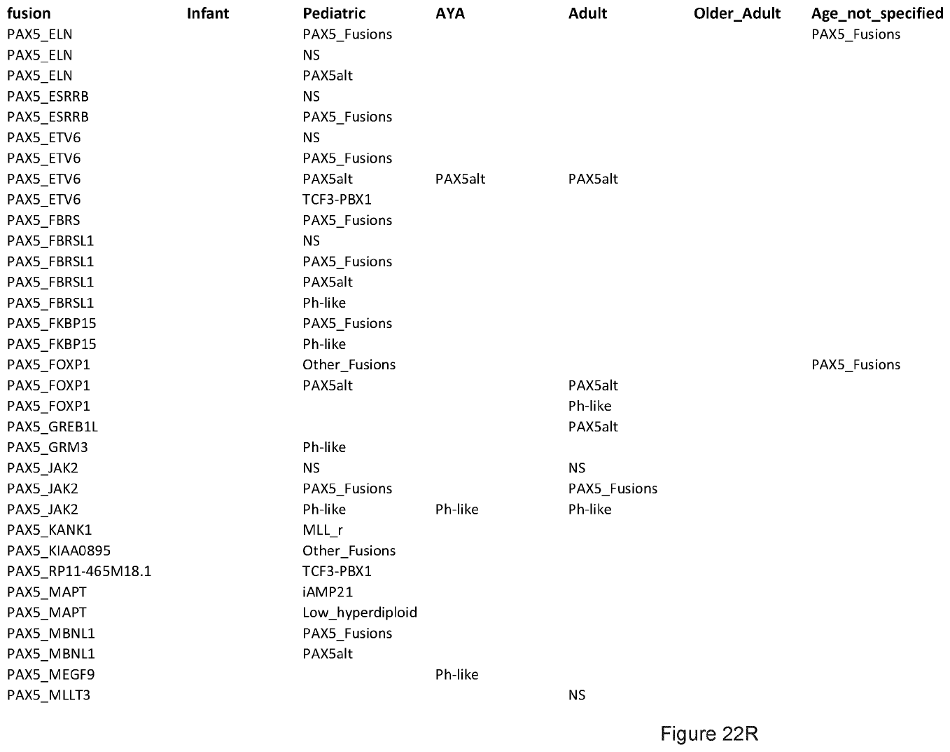

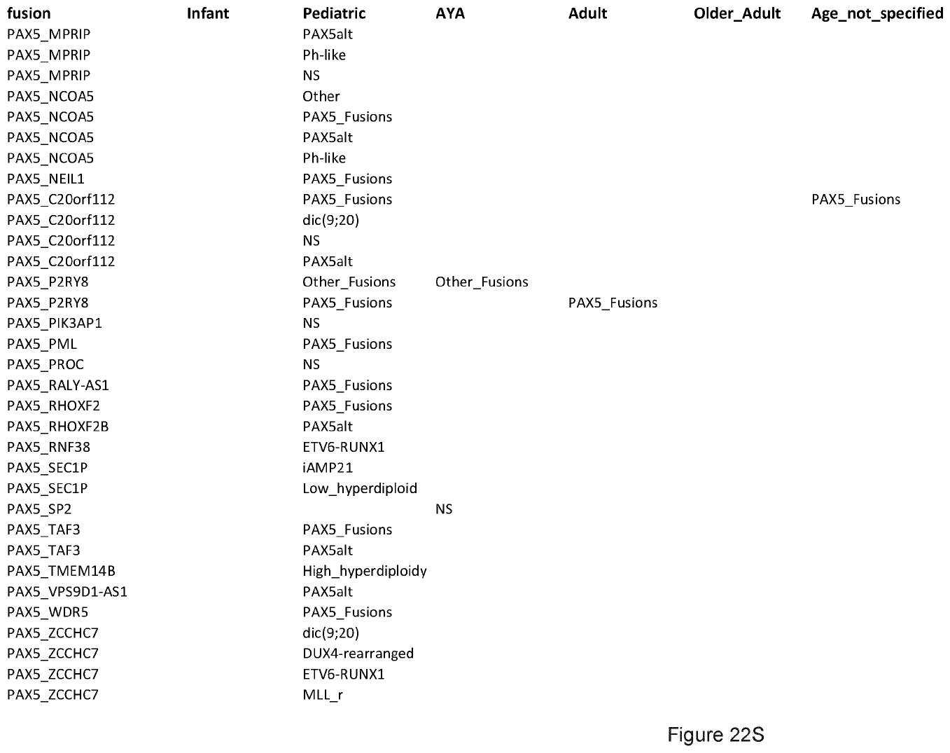

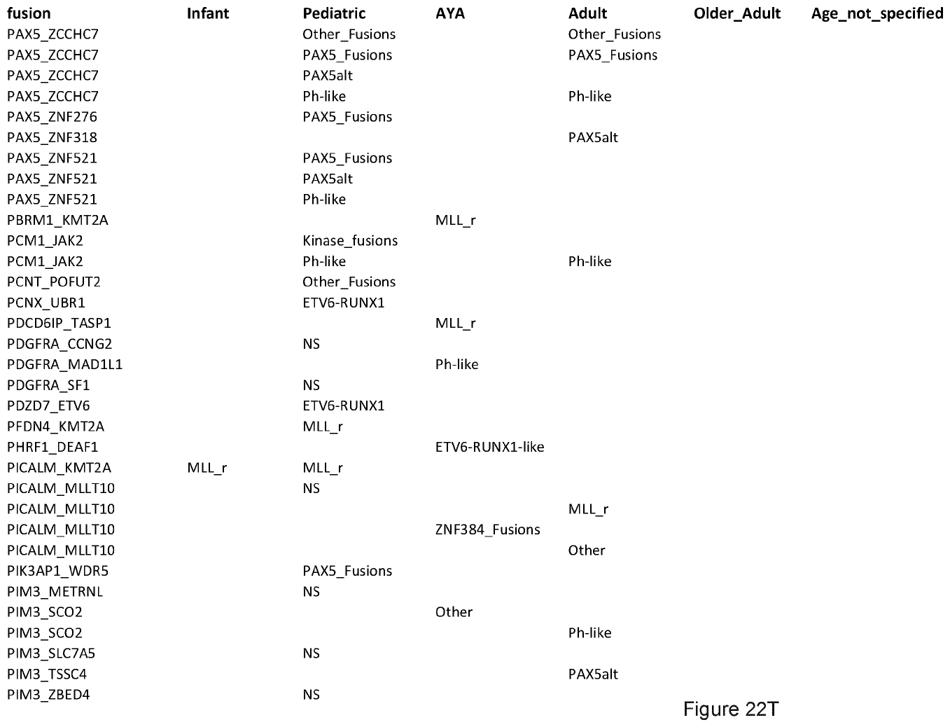

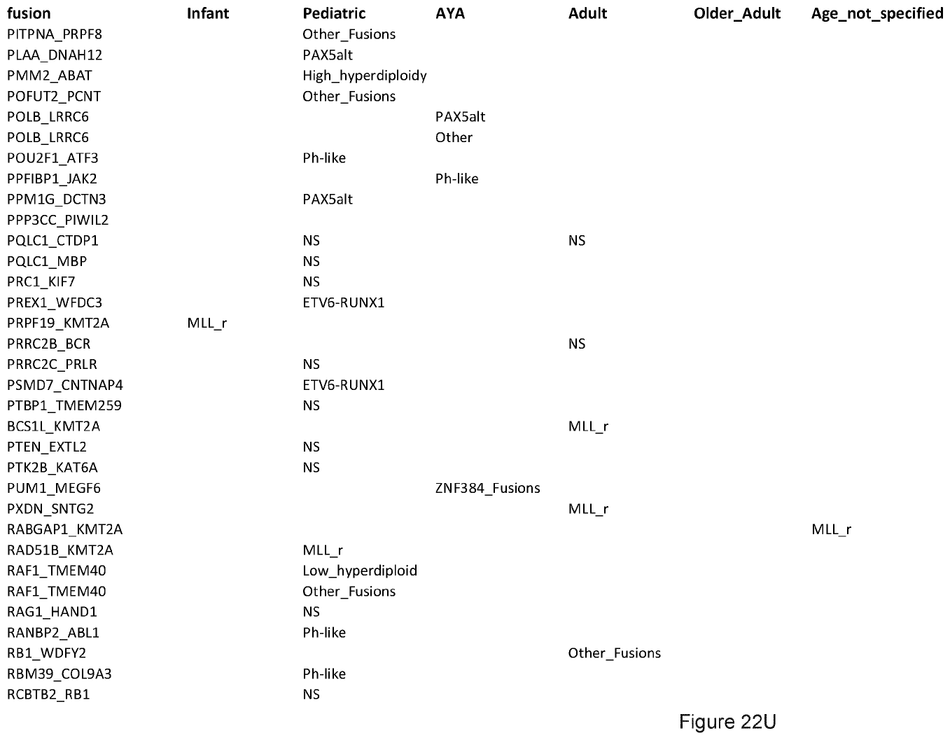

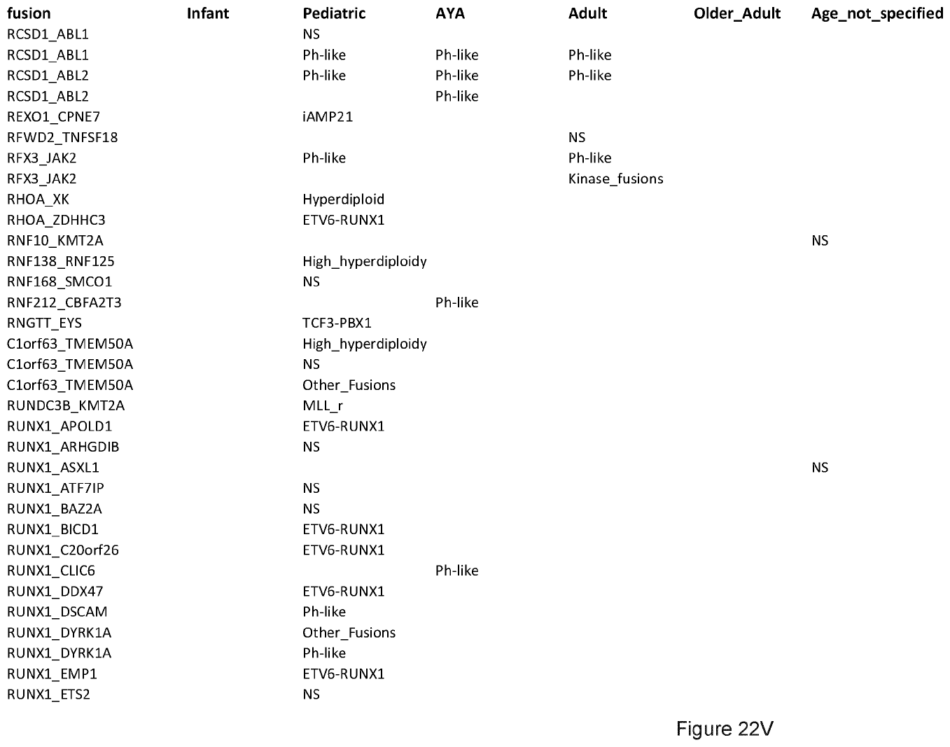

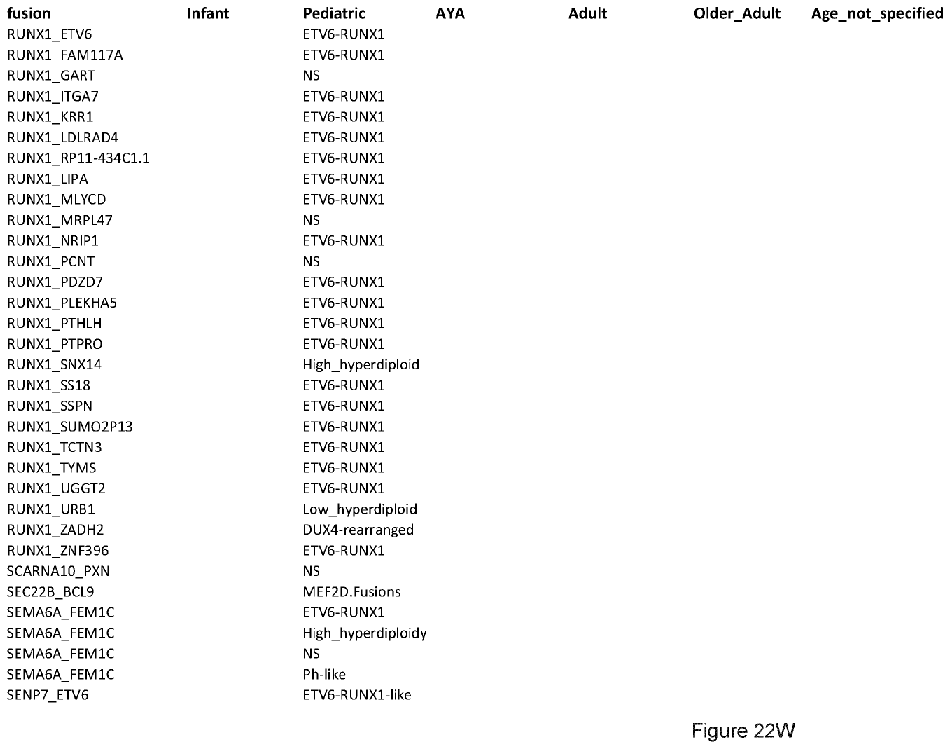

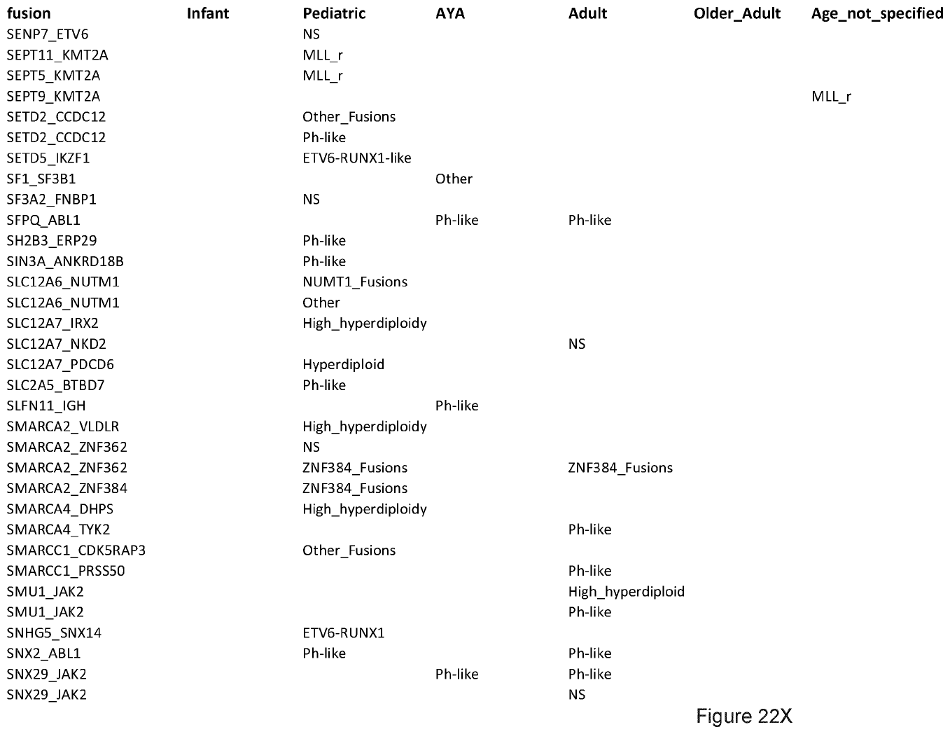

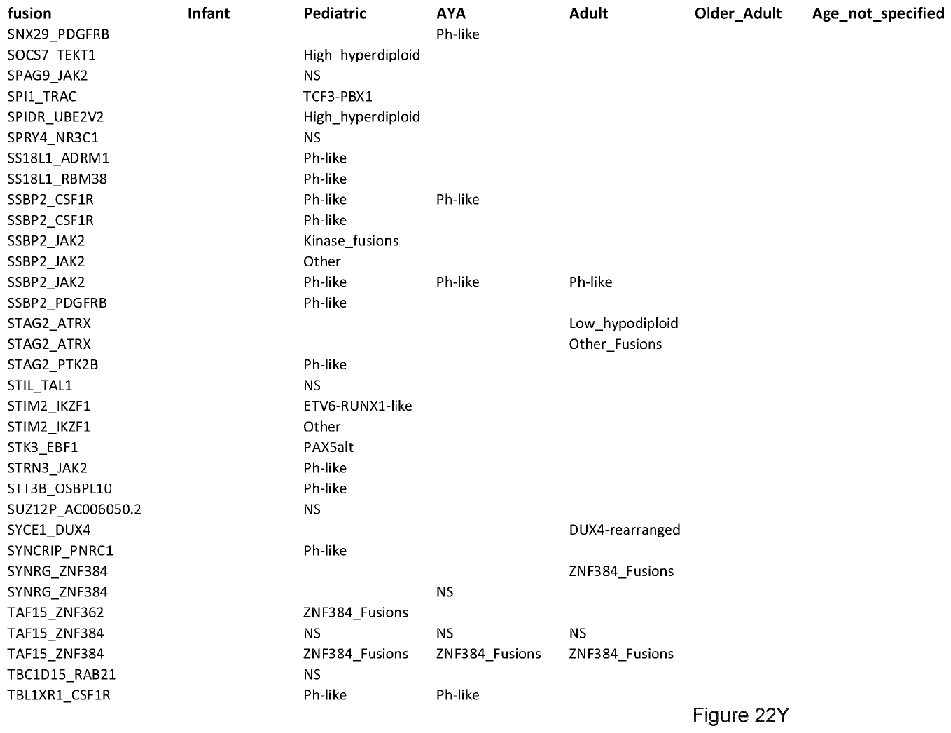

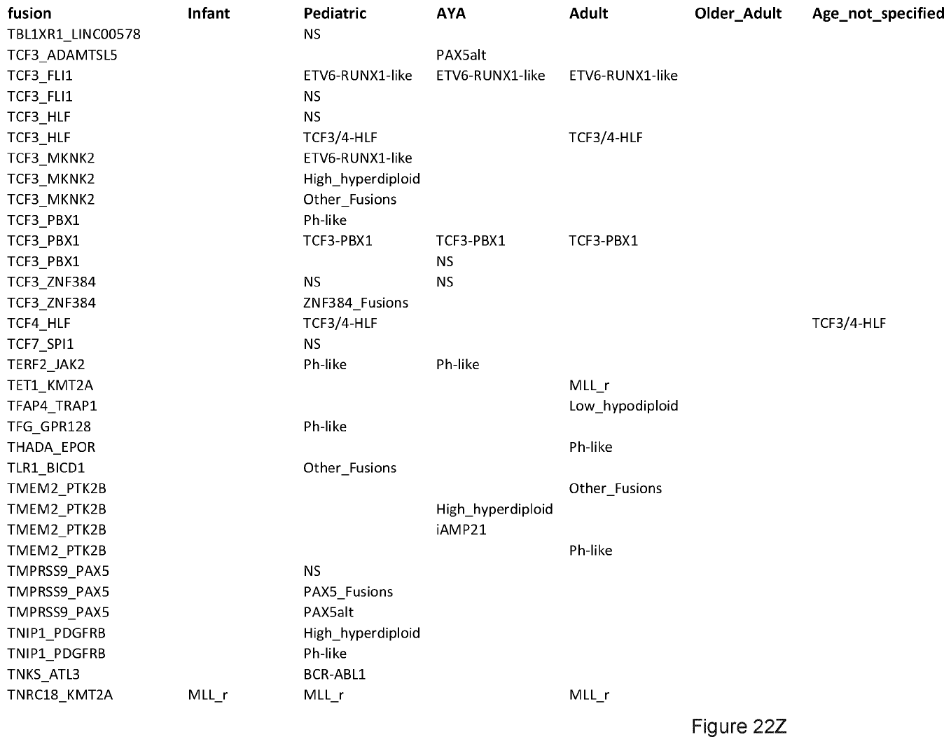

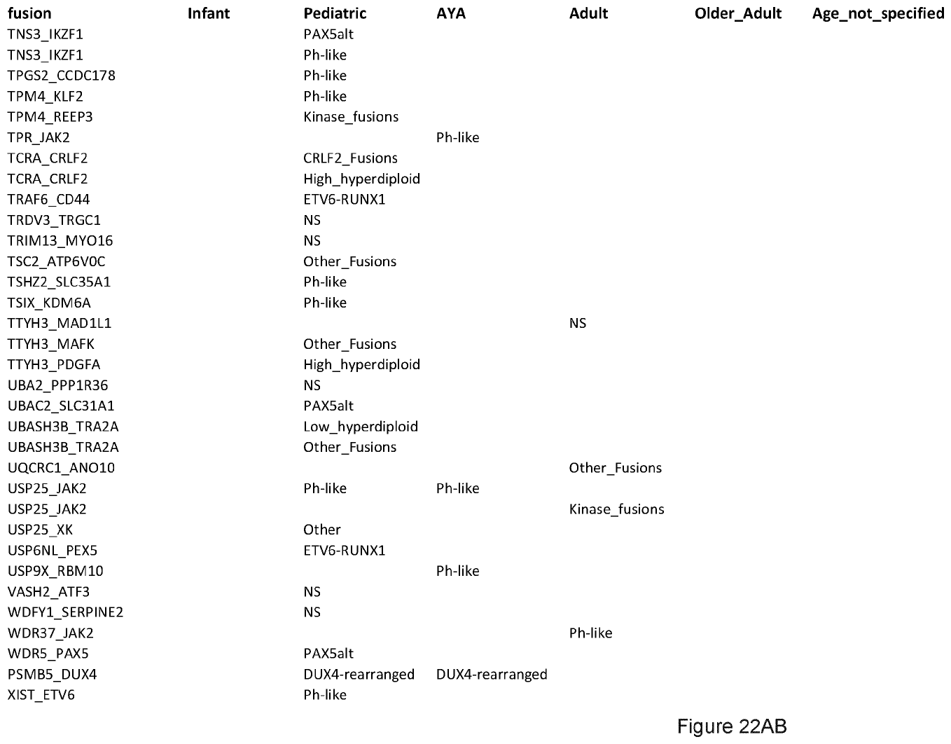

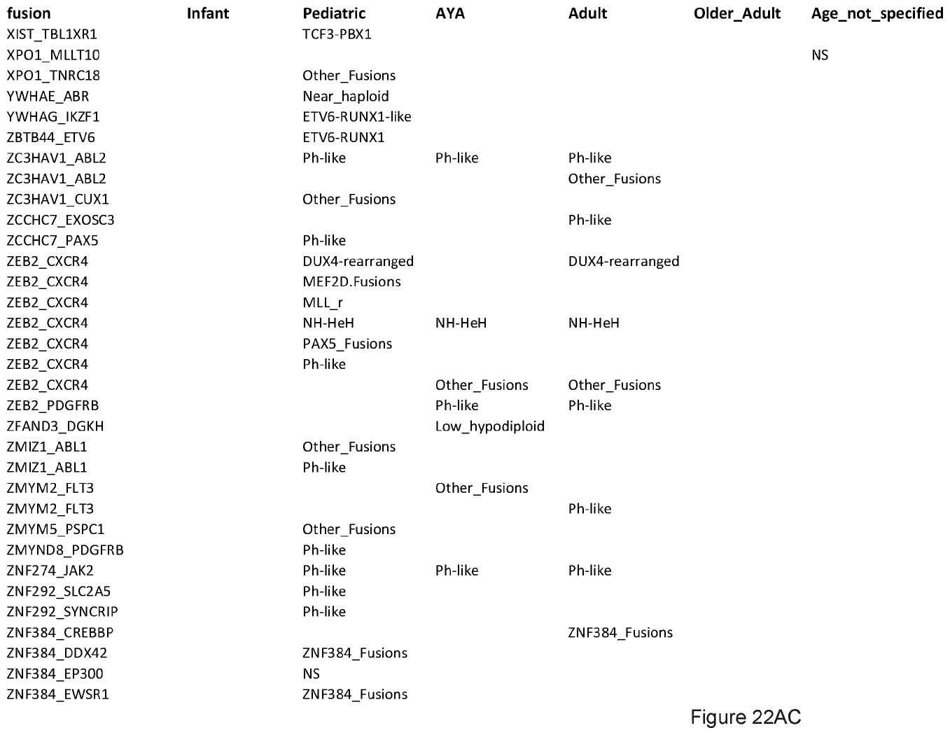

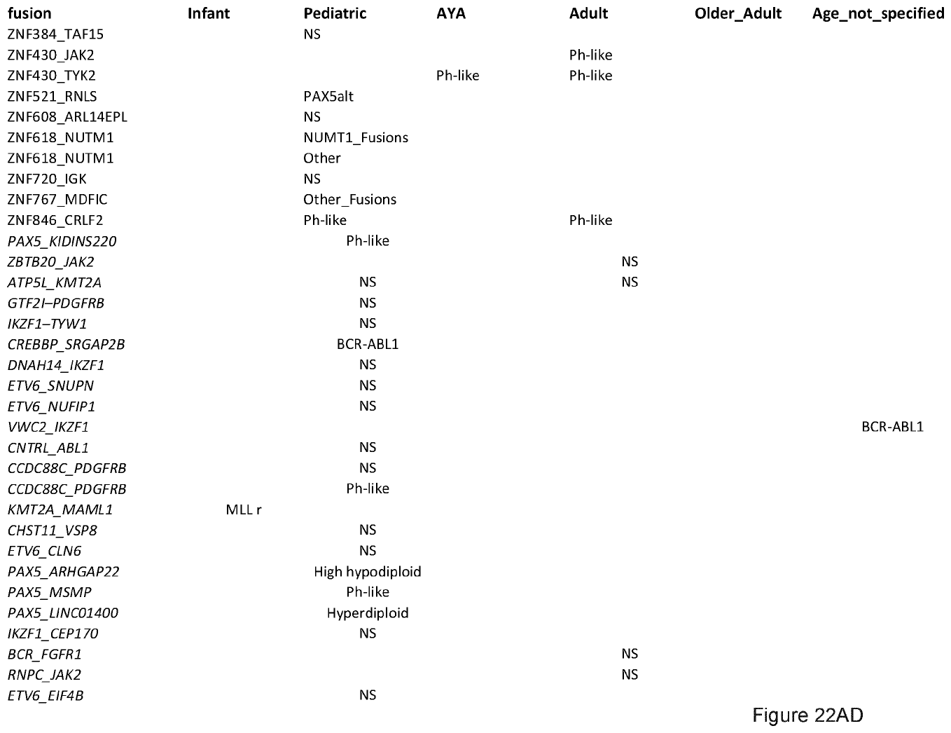

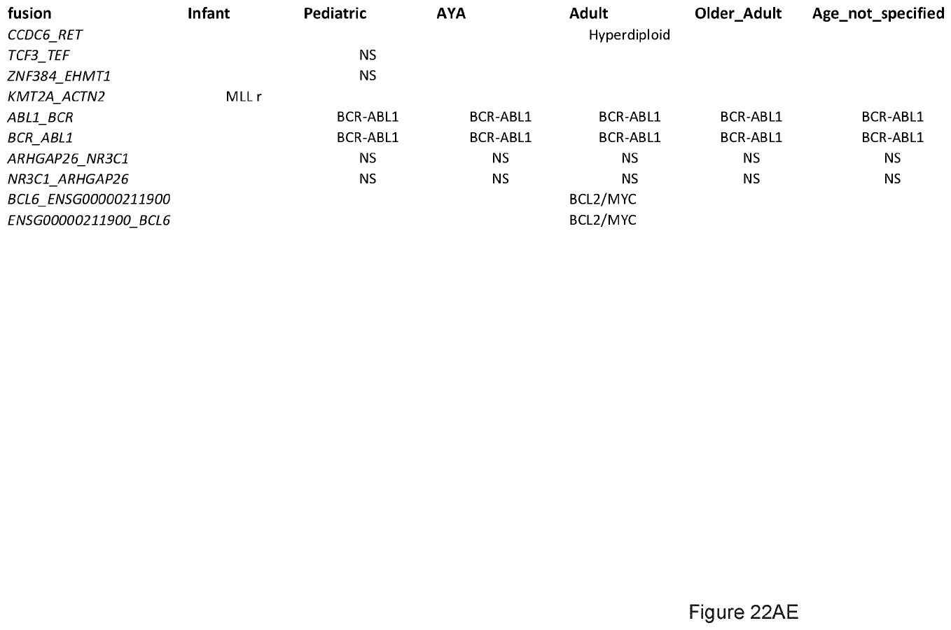

FIG. 22(A-AE). B-ALL fusion database. List of fusion transcripts described in B-ALL literature (see method section) were reported according to age [Infant (<1 year); Pediatric; AYA (Adult, Yung Adults); Adults; Elderly patients (Older Adults); Age Not Specified (NS)] and according B-ALL subclassification (if reported) in the following categories: MLL-rearranged, ETV6-RUNX1, TCF3-PBX1, BCR-ABL1, Ph-like, High hyperdiploid, Low hyperdiploid, Hyperdiploid, Low hypodiploid, Near haploid, NH-HeH (Near haploid-HighHyperdiploid), DUX4-rearranged, ZNF384 Fusions, MEF2D Fusions, PAX5alt, PAX5 Fusions, PAX5 P80R, ETV6-RUNX1-like, MLL-like, iAMP21, BCL2/MYC, CRLF2 Fusions, NUMT1 Fusions, Kinase fusions, TCF3/4-HLF, dic(9,20), ERG Del, CEBPE Fusions, HLF, Other Fusions, NS (Not Specified).

FIG. 23a-b. Table showing gene expression levels of the genes involved in fusion events and their comparison to the average expression level in the group of cases without fusion (Ratio_fus_no_fus). The significance of the difference in the expression level is determined by the p_value<0.05(*),<0.005(**),<0.0005(***). na=no expression for absence of the gene in the panel. ND=not determined.

### DETAILED DESCRIPTION OF THE INVENTION

Within the meaning of the present invention, for “genetic fusion” or “fusion” or “fusion gene” it is intended a hybrid gene formed from two previously independent genes. It can occur as a result of different mechanisms as translocation, interstitial deletion, or chromosomal inversion.

The method of the invention allows to obtain a filtered fusion list comprising fusions identified in the subject or subjects with a high degree of reliability, avoiding errors and false positives.

The method of the invention advantageously allows to:
Identify fusions relating to a disease that are frequently missed by most common pipelines,

FIG. 21. Fusion gene partner read depth fusion plot comparisons between fused samples and samples without two gene any fusions: A) TFG-GPR128 fusions (samples 26 and 37). B) ZEB2-CXCR4 (samples 18, 25, 60, 62 and 66). Grey background represents retained part of fusion gene partners in samples with the fusion (Rearranged) and in all other Ph−/−/− non fused samples (Non-Rearranged).

FIG. 22(a-o). B-ALL fusion database. List of fusion transcripts described in B-ALL literature (see method section) were reported according to age [Infant (<1 year); Pediatric; AYA (Adult, Yung Adults); Adults; Elderly patients (Older Adults); Age Not Specified (NS)] and according B-ALL subclassification (if reported) in the following categories: MLL-rearranged, ETV6-RUNX1, TCF3-PBX1, BCR-ABL1, Ph-like, High hyperdiploid, Low hyperdiploid, Hyperdiploid, Low hypodiploid, Near haploid, NH-HeH (Near haploid-HighHyperdiploid), DUX4-rearranged, ZNF384 Fusions, MEF2D Fusions, PAX5alt, PAX5 Fusions, PAX5 P80R, ETV6-RUNX1-like, MLL-like, iAMP21, BCL2/MYC, CRLF2 Fusions, NUMT1 Fusions, Kinase fusions, TCF3/4-HLF, dic(9,20), ERG Del, CEBPE Fusions, HLF, Other Fusions, NS (Not Specified).

FIG. 23a-b. Table showing gene expression levels of the genes involved in fusion events and their comparison to the average expression level in the group of cases without fusion (Ratio_fus_no_fus). The significance of the difference in the expression level is determined by the p_value<0.05(*),<0.005(**),<0.0005(***). na=no expression for absence of the gene in the panel. ND=not determined.

### Detailed Description of the Invention

Within the meaning of the present invention, for “genetic fusion” or “fusion” or “fusion gene” it is intended a hybrid gene formed from two previously independent genes. It can occur as a result of different mechanisms as translocation, interstitial deletion, or chromosomal inversion.

The method of the invention allows to obtain a filtered fusion list comprising fusions identified in the subject or subjects with a high degree of reliability, avoiding errors and false positives.

The method of the invention advantageously allows to:
Identify fusions relating to a disease that are frequently missed by most common pipelines,
detect known and unknown fusions and monitor their dynamics across disease evolution,
improve the characterization and classification of patients
identify novel target for therapeutic intervention
support therapeutic decisions.

In particular, i) candidate fusions would be lost if only those detected by 3 or 4 tools were considered, ii) the integration of a “literature filter” (step d1) allows to retain important fusions that would be otherwise discarded from the analysis.

The method will be herein described more in detail.

Reference can be made to FIG. 1A for a representation of an exemplary embodiment of the method of the invention.

In step a) genomic raw sequencing data are obtained from a subject and/or from a group of subjects.

In an exemplary embodiment, a biological sample is obtained from a subject affected by the disease. The biological sample can be chosen from the skilled person depending on the disease.

For example for subjects affected by leukemia diseases the biological sample can be peripheral blood (PB) or bone marrow (BM).

Genomic RNA is extracted from the sample and sequenced with common used methods.

Raw sequencing data obtained are converted to FASTQ file format according to known methods.

In step b) sample FASTQ files are analyzed with at least three informatic tools able to identify genetic fusions from said genomic sequencing data. Such informatic tools are known in the field and available to the skilled person.

In a preferred embodiment, such tools are:
FusionCatcher
STAR-Fusion
RNA-Seq Alignment
TopHat Alignment

In a more preferred embodiment, such 4 tools are combined in step b).

These tools are known and available in the field.

For FusionCatcher (FC) reference can be made to ref. [18], for STAR-Fusion (SF) reference can be made to ref. [19].

RNA-Seq Alignment and TopHat Alignment are two Basespace applications commercially available from Illumina, San Diego, Calif., USA.

Manta (the RNA-Seq Alignment fusion caller) and STAR-Fusion take advantage of STAR aligner, while TopHat-Fusion is built to run on TopHat alignments, and FusionCatcher combines BLAT, STAR, Bowtie and Bowtie2 [19-21].

The reference ‘Homo sapiens UCSC hg19’ (RefSeq and Gencode gene annotations) can be used for all the aligners.

The 4 tool fusion outputs are combined in a first genetic fusion list, wherein fusions identified by at least one of said tools are present.

In an embodiment, this first fusion list is further elaborated by determining for each sample how many times a fusion is called according to one or more of the following rules:
different breakpoints of the same fusion are considered as a single event for that sample,
reciprocal fusion are considered as a single event for that sample,
it is checked if a fusion gene partner is called with an alias,
it is checked if at the same breakpoint locus is present more than one gene, in case of a same breakpoint but a different gene name it can be checked on Ucsc the possible compresence of two genes in the same locus (https://genome.ucsc.edu)).

These rules are simplified in the following scheme 1:

In step c) fusions detected by at least three of said tools, i.e. three or four tools, are retained from the list thereby obtaining a second list with the retained fusions.

In step d) fusions present in said first genetic fusion list detected only by one or two tools are retained and added in said second fusion list only if they meet at least one of the following criteria:

d1. fusions are known for the disease, all the other fusions identified by one single tool are discarded,

d2. for transcripts detected by two different tools, but not previously published in the literature regarding the disease, fusion is not marked “false positive” by Fusion Catcher,

d3. For fusions detected by two tools, the ones labeled as significant for at least one of the three following events: a) Manta positive score combined with read/s positivity in the other tool, b) fusion positive comments in “FusionCatcher summary candidate fusion” output, c) EBF1 and ERG gene read-throughs (FusionCatcher) are retained.

For criteria d1 it is intended that the genetic fusion is already known as associated with the disease, for example from the literature regarding the disease. This step can be carried out by executing a research in the published scientific literature, for example using the Pubmed database from NIH (https://pubmed.ncbi.nlm.nih.gov/).

For criteria d2 it is intended that a fusion is selected if not marked as “false positive” in FusionCatcher summary candidate fusion output file.

For criteria d3 it is intended that for fusions detected by two tools, the ones labeled as significant for at least one of the three following events: a) Manta positive score (Manta attributes a score >0 in the Manta potential fusion output) combined with 21 junction read in the other tool that detected the fusion, b) fusion positive comments in “FusionCatcher summary candidate fusion” output (e.g. already known fusion, reciprocal fusion), c) EBF1 and ERG gene read-throughs (FusionCatcher) are retained.

In a preferred embodiment, all criteria d1, d2 and d3 should be satisfied in order to insert the fusion in said second fusion list.

In a preferred embodiment, all criteria d1, d2 and d3 should be satisfied and criteria d1 is considered as first, d2 as second and d3 as third criteria.

In a preferred embodiment, this second fusion list is further elaborated according to one or more of the following criteria:
reject fusions presents in run controls.
check if fusion described in normal samples are also already described in the disease. If fusion is described in the disease it is retained
check if a fusion is frequently called in the samples.

The filtered second fusion list obtained at the end of step d) can optionally be further integrated comparing the fusions with one or more of the databases of genetic fusions (step e) in order to annotate if each specific fusion was reported in other diseases, such as similar diseases, and/or in normal samples.

Such databases are known and available to the skilled person.

For example some or all of the following public fusion databases can be used:

i) tumor fusion gene data portal (https://www.tumorfusions.org/),

ii) COSMIC (https://cancer.sanger.ac.uk/cosmic/fusion),

iii) ChimerKB (http://www.kobic.re.kr/chimerdb/chimerkb),

iv) Mitelman Database of Chromosome Aberrations and Gene Fusions in Cancer (https://mitelmandatabase.isb-cgc.org/mb_search),

v) Fusion Gene annotation DataBase (https://ccsm.uth.edu/FusionGDB/)

In step e) fusions detected in normal samples (run healthy controls and/or described in literature and/or fusion databases) are rejected, but if described in disease literature are annotated as “normal” and considered separately to allow to the operator the decision if filter these cases.

In a preferred embodiment the method is used in a subject affected by B-ALL.

In this embodiment, in step d1 the database herein enclosed as FIG. 22 is used to check if fusions are already reported in B-ALL literature. If a fusion is reported in B-ALL literature cases, the fusion is retained. If a fusion is reported in B-ALL literature cases and in normal samples, the fusion is annotated as normal and considered separately to allow to the operator the decision if filter these cases.

This database was obtained as follows. Using Medline/Pubmed, literature on B-ALL from 2013 to 2019 was revised to create a B-ALL fusion database to use in the method of the invention as “literature retention filter”. Four keywords combination was used: “fusion AND acute lymphoblastic leukemia”, “transcriptome AND acute lymphoblastic leukemia”, “genomic landscape AND acute lymphoblastic leukemia” and “genomic profiling AND acute lymphoblastic leukemia”. Additionally using Medline/Pubmed, literature on B-ALL from 2019 to 2021 was revised to create a B-ALL fusion database with “fusion AND acute lymphoblastic leukemia” keywords combination. A more of 730 fusion list described in B-ALL at different ages was obtained. Considering B-ALL subgroup annotations and described reciprocal fusions a list of 967 fusion transcripts was obtained.

The method of the invention can be used to identify a genetic fusion associated with a subject affected by any disease. Preferably the disease is a cancer, more preferably a solid or hematological cancer, even more preferably B-ALL.

In an embodiment, the method is used in a B-ALL patient classified according to one of the following genomic alterations:
t(1,19)
t(4,11)
t(9,22)/Ph+
Ph−/−/−

In an embodiment, the method is used in a subject affected by acute myeloid leukaemia or acute lymphoblastic leukaemia, in particular the subtype wherein a MLL gene fusion is involved.

In other embodiments, the method is used in one of the following haematological tumors:
T-ALL
B-Cell Lymphoblastic Lymphoma
T-Cell Lymphoblastic Lymphoma
High grade Lymphoma
Lympho/myeloid acute leukemia
Mieloid leukemias, in particular acute myeloid leukemia, essential thrombocythemia, myelodysplastic syndrome, hypereosinophilic syndrome

In other embodiments, the method is used in one of the following solid tumors:
esophageal carcinoma
sarcomas
lynch syndrome
skin carcinoma
breast cancer

In an embodiment, the method is repeated on the same subject to evaluate progression of the disease, i.e. to verify if additional fusions were generated.

The method of the invention allows to detect gene fusions which can be used to classify the subject and to identify alternative therapeutic option.

The invention further provides a method to help to classify a subject affected by a disease into a known subtype of said disease comprising using the method above disclosed.

In particular, the fusion list obtained with the method can be compared to information known in the literature regarding the disease to classify the subject into a known subclass or subtype or subgroup of the disease.

In an embodiment, the invention provides a method to help to classify an adult B-ALL subject that is negative for t(9,22), t(4,11) and t(1,19) translocations (Ph−/−/− B-ALL subjects) into a known B-ALL subgroup comprising using the method above disclosed.

The invention further provides a method to identify a therapeutic treatment for a subject affected by a disease comprising performing the method of the invention in a sample from said subject in order to obtain a fusion list from said subject and select a suitable treatment based on said list. The selection of the suitable treatment can be made using common general knowledge in the field.

For example with regard to the disease B-ALL, reference for suitable treatments can be made to Table 5 below and/or to FIG. 5. Indeed, several pharmacological inhibitors targeting 30 out of 43 gene fusions are known. Some of these inhibitors have been experimentally tested in ALL samples and are currently known for their efficacy such as TKi against ABL-class fusions, while other fusions have been predicted as potential targets of commercially available inhibitors (e.g. Ruxolitinib, HDACi, BCL6i).

In an exemplary embodiment, the subject is a B-ALL Ph-like patient and if fusions involving ABL-class genes (ABL1, ABL2, CSF1R, and PDGFRB) is identified ABL1 tyrosine kinase inhibitors (TKIs) can be used as treatment. In another embodiment, JAK2 fusion proteins or truncating rearrangements of the erythropoietin receptor (EPOR) is identified in the subject and ruxolitinib can be used as treatment.

Indeed, B-ALL Ph-like is a high-risk subtype characterized by genomic alterations that activate cytokine receptor and kinase signaling. Kinase more frequent alterations include fusions involving ABL-class genes (ABL1, ABL2, CSF1R, and PDGFRB) sensitive to ABL1 tyrosine kinase inhibitors (TKIs) or rearrangements that create JAK2 fusion proteins or truncating rearrangements of the erythropoietin receptor (EPOR) that are sensitive to ruxolitinib in vitro [49]. ABL-class and JAK genes are also promiscuous genes, and their fusion detection is crucial for classification and for alternative therapeutic option identification.

The method of the invention can be implemented in a data processing device, such as a computer.

Any suitable data processing device can be used for implementing the method of the invention.

A data processing device comprising means adapted for carrying out the steps of the method of the invention is also within the scope of the present invention.

A computer program comprising instructions which, when the program is executed by a computer, cause the computer to carry out the steps of the method of the invention is also within the scope of the present invention.

A computer-readable storage medium comprising instructions which, when executed by a computer, cause the computer to carry out the steps of the method of the invention is also within the scope of present invention.

The present invention will be now illustrated by the following examples.

### EXAMPLES

### Material and Methods

### Patients Characteristic and Inclusion Criteria

The study included 63 RNA samples [extracted from total mononuclear cells isolated from peripheral blood (PB) or bone marrow (BM) samples of Ph−/−/− B-ALL samples and from the PB of adult healthy donors] of 57 B-ALL adult “Ph−/−/−” B-ALL patients at different time points and three healthy donors. The samples included in the study were defined “Ph−/−/−” B-ALL when negative for t(9,22)(q34,q11), t(4,11)(q21,q23) and t(1,19)(q23,p13) translocations using conventional methodologies. Forty samples were collected at the time of diagnosis, 12 at relapse, for 5 cases the inventors sequenced both diagnosis and relapse and for one case the inventors analyzed two different relapses (Table 1).

TABLE 1
Summary of fusion detection in 63 samples of 57 Ph—/—/—
B-ALL adult patients and their characteristics.

| Pt | | | | |
|---|---|---|---|---|
| N | Phase | Sample | Fusion Summary | Karyotype |
| 1 | D P | 8 | PAX5-ZCCHC7 | 46, XY [15]; 46, XY, t (2; 16) (p15; q22) [5] |
| | | | THADA-CDH1 | |
| | R P | 27 | PAX5-ZCCHC7 | 46, XY |
| | | | TET3-ETV6 | |
| 2 | D | 9 | PAX5-ZCCHC7 | 46, XY |
| 3 | D P | 10 | | 46, XX, del (9) (13p22p) [12]/46, XX [8] |
| | R P | 20 | ARHGAP26-NR3C1 | NV |
| | | | RUNX1-RCAN1 | |
| 4 | D P | 11 | | NV |
| | R P | 56 | | 46, XY |
| 5 | D | 12 | RCSD1-ABL2; | 46, XY |
| | | | ABL2-RCSD1 | |
| 6 | D | 13 | P2RY8-CRLF2 | 46, XY |
| 7 | D | 14 | | NA |
| 8 | D | 15 | | 46, XX |
| 9 | D | 17 | DUX4-IGH | 46, XY |
| 10 | D P | 18 | (ENSG00000231231) | 46, XY |
| | | | LINC01423-ERG | |
| | R P | 52 | (ENSG00000231231) | NV |
| | | | LINC01423-ERG | |
| 11 | R | 19 | TCF3-ZNF384 | 45, XY, −7 [9]/45XY, −7, del (9) (p11) [3]/46, XY [12] |
| 12 | D | 21 | TAF15-ZNF384 | 46, XX, t (12; 17) (p13; q21) [8]/46, XX [12] |
| 13 | R | 22 | RCSD1-ABL1; | 46, XY |
| | | | ABL1-RCSD1 | |
| 14 | D | 23 | EBF1-LINC02227 | 46, XY |
| 15 | D | 24 | | 46, XX |
| 16 | R | 25 | ZEB2-CXCR4 | NA |
| 17 | D | 26 | ARL11-RB1 | 46, XY |
| | | | TFG-GPR128 | |
| | | | (ADGRG7) | |
| 18 | D | 28 | UBXN4-CRCX4 | NA |
| | | | ARHGAP26-NR3C1 | |
| 19 | D | 29 | KMT2A-MLLT1 | NA |
| 20 | D | 30 | | NV |
| 21 | D | 31 | | Hyperdiploid |
| 22 | D | 32 | KMT2A-MLLT1 | 47, XY, del (3) (q13), +6, t (11; 19) (q23; p13) [4]/46, |
| | | | | XY [6] |
| 23 | D P | 33 | | NV |
| | R P | 81 | ARID5B-KMT2D | Complex karyotype [3]/46XY [1] |
| 24 | D | 34 | NONO-TFE3 | 46, XX |
| 25 | D | 35 | ARHGAP26-NR3C1 | 46, XX |
| | | | CBFA2T3-SLC7A5 | |
| | | | ZFPM1-SLC7A5 | |
| | | | NCOR2-BCL7A | |
| 26 | D | 36 | PAX5-ZCCHC7 | NA |
| 27 | D | 37 | PAX5-ETV6 | 46, XY |
| | | | TFG-GPR128 | |
| | | | (ADGRG7) | |
| 28 | D | 38 | | 46, XY |
| 29 | D | 39 | | 46, XY |
| 30 | D | 40 | | 46, XY |
| 31 | D | 41 | | 47, XX, +8 [16]/46, XX [4] |
| 32 | D | 42 | EP300-ZNF384; | 46, XX |
| | | | ZNF384-EP300 | |
| | | | CBFA2T3-SLC7A5 | |
| | | | PTMA-CXCR4 | |
| 33 | D | 43 | EP300-ZNF384 | NV |
| | | | ARHGAP26-NR3C1 | |
| 34 | D | 44 | | NA |
| 35 | D | 45 | | 46, XX |
| 36 | D | 46 | PPP3CC-CCAR2 | 46, XY, t (2; 16) (q13; q22), t (11; 14) (q23; q32) |
| | | | | [3]/46, XY [17] |
| 37 | D | 50 | | 68-71, |
| | | | | XXY, −X, −X, −Y, +1, +2, +5, −7, −9, −10, +11, −13, −18 |
| | | | | [9]/46, XY [11] |
| 38 | R | 51 | PAG1-PLAG1 | 46, XY |
| | | | ARHGAP26-NR3C1 | |
| 39 | D | 35 | IGH-MYC | 46XX, der (6) t (1; 6) (q11; q11), t (8; 14) (q24; q32), |
| | | | | del (9) (q21), add (13) (q34) |
| | | | FLI1-ZBTB16 | |
| | | | CRTC1-SUGP2 | |
| | | | ALDH3B1-TFDP1 | |
| | | | CPSF6-BRD1 | |
| | | | OAZ1-DOT1L | |
| 40 | R | 54 | MEFD2-BCL9 | NA |
| 41 | D | 55 | CXCR4-GNAS | High hiperdiploid (>50Chrs) |
| 42 | D | 57 | IKZF1-IGKV5-2 | NA |
| | | | P2RY8-IGH | |
| 43 | R | 58 | EP300-ZNF384; | NA |
| | | | ZNF384-EP300 | |
| 44 | D | 60 | NR3C1-ARHGAP26 | 46, XX |
| | | | ZEB2-CXCR4 | |
| 45 | D | 61 | (IGHJ5 & J6) | 47, XY, +4, −6, i (6) (p10), add (11) (q23), +mar [8]/46, |
| | | | IGH-BCL6 | XY [12] |
| 46 | D | 62 | ZEB2-CXCR4 | High hiperdiploid (>50Chrs) |
| 47 | D | 63 | ZNF362-SMARCA4 | 46, XX |
| 48 | D | 64 | TCF3-ZNF384; | 46, XX |
| | | | ZNF384-TCF3 | |
| 49 | D | 65 | NUMA1-CSF1R | 46, XY |
| 50 | R | 66 | ZEB2-CXCR4 | 54-56, XY, +der (X) del |
| | | | | (Xq22)?, +der (22), +8, +10, +11, +21, +2-4mar |
| 51 | R | 67b | | 46, XX, der (4), del (4) (q21q35), del (7) (q22q36), der |
| | | | | (10q) add (10) (q23), der (X)add (X) (q26) [2]/46, XX [22] |
| | R2 | 68 | MARCH8-NCOA4 | NA |
| 52 | R | 79 | | Complex karyotype |
| 53 | R | 82 | | NA |
| 54 | R | 84 | | NA |
| 55 | D | 86 | NCOR2-BCL7A | 47, XXY |
| 56 | D | 87 | | 46, XY |
| 57 | R | 92 | | NA |

D = Diagnosis, D P = Diagnosis Paired, R = Relapse, R P = Relapse Paired and R2 = Second Relapse.
Pt = patient.
NA = Not Available.
NV = Not evaluable.

The study was approved by the local Institutional Review Boards. Informed consent was obtained in accordance with the Declaration of Helsinki.

### Targeted RNA Sequencing and Fusion Prediction

Genomic RNA was extracted, using RNeasy Mini Kit (QIAGEN, Hilden, Germany), from total mononuclear cells isolated from peripheral blood (PB) or bone marrow (BM) samples of Ph−/−/− B-ALL samples and from the BM (n. 3) and PB (n. 5) of adult healthy donors (following sign informed consent). Additionally we extracted RNA from BM CD34+(n. 3) and cord blood CD34+(n. 4) healthy donors. RNA libraries were prepared using the TruSight RNA Pan-Cancer Panel Kit (Illumina, San Diego, Calif., USA), following the manufacturer's protocol. The panel is enriched for 1385 cancer-associated genes and permit fusion detection if at least one of the two gene partners is present in the panel genes. Paired-end RNA-sequencing was performed (Reagent Kit v3-150 cycles, MiSeq, Illumina, San Diego, Calif., USA) and raw sequencing data were converted to FASTQ file format and analyzed combining FusionCatcher (FC[18]), STAR-Fusion (SF) and two Basespace applications [RNA-Seq Alignment v.1.1.0 (RSA) and TopHat Alignment v.1.0.0 (THA), Illumina, San Diego, Calif., USA]. Manta (the RNA-Seq Alignment fusion caller) and STAR-Fusion take advantage of STAR aligner, while TopHat-Fusion is built to run on TopHat alignments, and FusionCatcher combines BLAT, STAR, Bowtie and Bowtie2[19-21]. The reference ‘Homo sapiens UCSC hg19’ (RefSeq and Gencode gene annotations) was used for all the aligners.

The inventors retained fusions detected by at least three tools and the inventors introduced two further criteria to retain or reject fusions detected by two or one tool (FIG. 1A). In order of priority, the inventors firstly retained fusions (and their reciprocal transcripts) that were already reported in ALL literature and included in the list of known fusions created as described below. All the other fusions identified by one single tool were discarded. Second, for transcripts detected by two different tools, but not previously published in ALL, the inventors excluded fusion marked “false positive” by Fusion Catcher. Third, of the remaining fusions detected by two tools, the inventors retained the ones labeled as significant for at least one of the three following events: a) Manta positive score, b) fusion positive comments in “FusionCatcher summary candidate fusion” output, c) EBF1 and ERG read-throughs (FusionCatcher).

Using Medline/Pubmed, the inventors revised literature on B-ALL from 2013 to 2019, to create a B-ALL fusion database to use in their pipeline for “literature retention filter”. The inventors used four keywords combination: “fusion AND acute lymphoblastic leukemia”, “transcriptome AND acute lymphoblastic leukemia”, “genomic landscape AND acute lymphoblastic leukemia” and “genomic profiling AND acute lymphoblastic leukemia”. Additionally using Medline/Pubmed, literature on B-ALL from 2019 to 2021 was revised to create a B-ALL fusion database with “fusion AND acute lymphoblastic leukemia” keywords combination and the inventors obtained a more of 730 fusion list described in B-ALL at different ages was obtained. Considering B-ALL subgroup annotations and described reciprocal fusions a list of 967 fusion transcripts was obtained (FIG. 22A-AE).

In addition, the definitive filtered fusion list was further integrated with five public fusion database: a) https://www.tumorfusions.org/, b) https://cancer.sanger.ac.uk/cosmic/fusion, c) http://www.kobic.re.kr/chimerdb/chimerkb, d) https://mitelmandatabase.isb-cgc.org/mb_search, e) https://ccsm.uth.edu/FusionGDB/ in order to annotate if each specific fusion was reported in other solid and hematological tumors (excluding ALL) and/or in normal samples.

Finally, the inventors used a Venn diagram (http://bioinformatics.psb.ugent.be/webtools/Venn/) to illustrate the logical relationships between fusions and mining-tools (FIG. 1B).

### Fusion Gene Partner Differential Gene Expression Analysis

Gene expression was calculated with Cufflinks software (http://cole-trapnell-lab.github.io/cufflinks/, v2.2.1), using as input the same STAR alignments employed by STAR-Fusion (STAR v2.5.2b[19,22]). Gene and isoform expression is calculated in terms of FPKM (fragments per kilobase of exon model per million reads mapped). A confidence interval and a quality score of the estimated value is also given for each gene by the software.

The average expression of the gene of interest in the group of samples in which the fusion is present was compared with the expression of the same gene in the pool of samples in which the gene is not fused. For example, to perform differential expression of the gene A in the fusion A-B the inventors consider the group “fused” as composed by the samples where the fusion A-B (or B-A) is present, and the group of “non-fused” as composed by samples where the gene A is not fused with any gene.

The comparison between the groups “fused” and “non-fused” is performed by calculating the fold change (expression log ratio) of gene A in the two groups.

Fold change is estimated by calculating for each gene the log ratio (T) between the average expression in “fused” (R) and “not-fused” (G) groups:

T=R−G

R=(Σi fi)/N, i=1, 2, . . . N samples in “fused”

G=(Σi fi)/M, i=1, 2, . . . M samples in “not-fused”

Where fi represents the natural logarithm of FPKM value of the gene under consideration in sample “i”. The log ratio spans both positive and negative values: T<0 indicates under-expressed fused genes and T>0 indicates over-expressed fused genes. If the gene involved in the fusion is not part of the panel, its expression value is not available (or reliable) and T cannot be determined. Panel genes which are unexpressed (FPKM<=10\hat{ }(−5)) have all been shifted to value FPKM=10\hat{ }(−5), to evaluate the fold change between the groups.

Despite in general this is not the most appropriate method to estimate differential expression between two conditions, the expression log ratio is preferable for this specific task. In fact, the two groups of samples “fused/non-fused” are defined on the bases of a single gene only (A). Instead, bioinformatic tools commonly used to assess differential expression, for example DeSeq or Cuffdiff[23], normalize expression values at the level of the entire pool of samples and by considering all available genes. This could not account for further fusion events present in the samples, thus including in the same pool the expression levels of both fused and non-fused genes without any distinction.

The significance of up or down regulation of a gene in a specific sample is quantified through the Z-score of the log expression across all the samples, to determine the significance of the deviation respect to the global average. The Z-score is performed to determine the significance of the deviation respect to the global average, and it is calculated respect to the logarithms of the FPKM values for better controlling overdispersion of the expression levels, and for assuming a normal distribution of the FPKM log values in the computation of p-values. Then, the corresponding p-value is computed under a normal distribution constrain. The inventors consider three level of significance of the Z-score p-value: * <0.05, ** <0.005 and *** <0.0005 (FIG. 23a-b).

### Fusions Validation Using Conventional and Molecular Cytogenetic Analyses

To confirm the gene fusions identified by transcriptome sequencing approaches, the inventors performed Chromosome Binding Analysis (CBA) and Fluorescent In Situ Hybridization (FISH). CBA was performed on BM cells as previously reported[24]. FISH analysis was carried out on fixed nuclei and previously GAW-banded metaphases obtained from CBA technique, according to manufacturer's recommendations. Dual color dual fusion FISH was performed with BAC clones RP11-17L5, RP11-354N7, RP1l-81M8, RP11-433J6, RP11-980B20, RP11-138P4, LSI ETV6 (TEL)-RUNX1 (AML1) ES Dual Color Translocation Probe Set (Vysis, Abbott Molecular, IL, USA) and BCR/ABL1/ASS1 Tri-Color DF FISH Probe (Vysis). Further details are reported in Table 2.

TABLE 2
FISH probe details.

| Gene | Probe and label | Start-End position |
|---|---|---|
| RCSD1 | RP11-138P14-Green | chr1:167586570-167756733 |
| THADA | RP11-17L5-Green | chr2:43428488-43583624 |
| TET3 | RP11-980B20-Orange | chr2:74076600-74264678 |
| ZNF384 | RP11-433J6-Orange | chr12:6709475-6885639 |
| CDH1 | RP11-354N7-Orange | chr16:68761062-68921293 |
| TCF3 | RP11-81M8-Green | chr19:559524-2400395 |
| ETV6 | LSI ETV6 | NA |
| | Spectrum Green* | |
| | LSI ALB1 Spectrum | |
| | Orange, LSI | |
| ABL1 | ASS/ABL1 Spectrum | NA |
| | Aqua/Spectrum | |
| | Orange* | |

NA: not applicable,
*these probes are included in two commercially available FISH Probe (Vysis). They were used in combination with RP11-980B20 and RP11-138P14 to create a specific dual color dual fusion probe to detect TET3-ZNF384 and RCSD1-ABL1 rearrangements.

BAC clones were selected according to the breakpoint position identified by RNA sequencing. BAC clones were marked in Spectrum Orange or Spectrum Green (Empire Genomics, New York, USA). The slides were counter stained with DAPI and analyzed using fluorescent-microscopes equipped with FITC/TRITC/AQUA/DAPI filter sets and the Genikon imaging system software (Nikon Instruments, Tokyo, Japan). At least 200 nuclei were analyzed for each sample.

### Fusions Validation Using Conventional Molecular Analyses

Reverse transcription (RT) was performed using SuperScript III Reverse Transcriptase or MultiScribe Reverse Transcriptase enzyme mix (Invitrogen and AppliedBiosystem). Sanger sequencing was performed on the fusion breakpoint region. Sequencing was performed using the BigDye Terminator V.3.1 Sequencing Kit (Applied Biosystems, Foster City, Calif., USA). The complete list of primers and annealing temperatures used in the study is reported in Table 3.

TABLE 3
Primer sequences for RT-PCR and for Sanger sequencing along with
amplicon size (bp) and annealing temperature (T °C.).

| Target gene | Primers sequences (5′-3′) | Size (bp) | Annealing T° (C) | SEQ ID N. |
|---|---|---|---|---|
| RUNX1 ex3F | GGAAAAGCTTCACTCTGACCA | 207 | 61.5 | SEQ ID N. 1 |
| RCAN1 ex2R | GGAGAAGGGGTTGCTGAAGT | 207 | 61.5 | SEQ ID N. 2 |
| PAG1 ex2F | TTTTCTCCTATTTCAGCAGTTGG | 119 | 60 | SEQ ID N. 3 |
| PLAG1 ex3R | ACTTTGATCTTAGCCAGTCCCA | 119 | 60 | SEQ ID N. 4 |
| PAG1 ex1F | CGGAGTTTGCAGAGG | 200 | 52 | SEQ ID N. 5 |
| PLAG1 ex3R | ACTTTGATCTTAGCCAGTCCCA | 200 | 52 | SEQ ID N. 6 |
| PPP3CC ex3F | GGATCACCTAGTAACACACGC | 161 | 61.5 | SEQ ID N. 7 |
| CCAR2 ex2R | CTGAGAAGTTGCGTCCCC | 161 | 61.5 | SEQ ID N. 8 |
| MARCH8 ex1UTR | GGAAGCTCGGACTAGTGATCC | 300 | 61 | SEQ ID N. 9 |
| NCOA4 ex4R | AAGACATTCCAGGTGACGG | 300 | 61 | SEQ ID N. 10 |
| NUMA1 ex26F | CCTCAACACACCCAAGAAGC | 230 | 61.5 | SEQ ID N. 11 |
| CSF1R ex12R | TGCCCTCATAGCTCTCGATG | 230 | 61.5 | SEQ ID N. 12 |
| IKZF1 ex7F | ACAGTGAAATGGCAGAAGACC | 164 | 61.5 | SEQ ID N. 13 |
| IGKV5-2 ex2R | CGCTGACATGAATGCTGGAG | 164 | 61.5 | SEQ ID N. 14 |
| CYFIP2 ex5F | GCTTGCCCCGACATGAGTAT | 299 | 56 | SEQ ID N. 15 |
| EBF1 ex7R | CCGCATGTCACGTGGGTT | 299 | 56 | SEQ ID N. 16 |
| UBXN4 ex1F | GAGACTACACACCGAGCGAG | 393 | 57.5 | SEQ ID N. 17 |
| CXCR4 ex2R | TCTTCACGGAAACAGGGTTC | 393 | 57.5 | SEQ ID N. 18 |
| ZFPM1 ex1F | CGCGGGTTCCATTGAGAAAA | 424 | 55.5 | SEQ ID N. 19 |
| SLC7A5 ex3R | AATGCCAGCACAATGTTCCC | 424 | 55.5 | SEQ ID N. 20 |
| CBFA2T3 ex1F | ATGCCGGCTTCAAGACTGA | 227 | 62.5 | SEQ ID N. 21 |
| SLC7A5 ex3R | AATGCCAGCACAATGTTCCC | 424 | 62.5 | SEQ ID N. 22 |
| NCOR2 ex1F | GAGTCTTTGAGGACACAGCC | 118 | 58 | SEQ ID N. 23 |
| BCL7A ex3R | ATCAACCTTGGGCTCCGTC | 118 | 58 | SEQ ID N. 24 |
| NCOR2 ex17F | TCCATGGAGCTGAATGAGAGT | 140 | 57 | SEQ ID N. 25 |
| BCL7A ex3R | ATCAACCTTGGGCTCCGTC | 140 | 57 | SEQ ID N. 26 |
| TAF15 ex6F2 | AGAGCACCTTCCTATGACCAGCCAGAC | 252 | 66.5 | SEQ ID N. 27 |
| ZNF384_ex4R3 | GCCAGACCACAGCCCTTCTCTGGCA | 252 | 66.5 | SEQ ID N. 28 |
| PAX5 ex9F | GGAGTCCCTACAGCCACCCTC | 135 | 62 | SEQ ID N. 29 |
| ZCCHC7e5R2 | GGGGGCTGGACAGGAATACAGGAGA | 135 | 62 | SEQ ID N. 30 |
| ZEB2 ex2F | TCTTATCAATGAAGCAGCCGATC | 203 | 60.5 | SEQ ID N. 31 |
| CXCR4 ex2R | GAGTAGATGGTGGGCAGGAA | 203 | 60.5 | SEQ ID N. 32 |
| ZEB2 ex1UTRF | AGCTGTTTCTTCGCTTCCAC | 225 | 60.5 | SEQ ID N. 33 |
| CXCR4 ex2R | GAGTAGATGGTGGGCAGGAA | 225 | 60.5 | SEQ ID N. 34 |
| IL7R_ex6F | TGCATGGCTACTGAATGCTC | 349 | 57 | SEQ ID N. 35 |
| IL7R_ex6R | GGACAGCGTTTGCCTAATGT | 349 | 57 | SEQ ID N. 36 |
| CRLF2_ex6F | CGCACGTCATGTTGAAAACT | 304 | 56.5 | SEQ ID N. 37 |
| CRLF2_ex6R | CCATCATAAGAGTGGGCATTG | 304 | 56.5 | SEQ ID N. 38 |
| JAK2_ex16F | CTCAATGCATGCCTCCAA | 341 | 62 | SEQ ID N. 39 |
| JAK2_ex16R | ACAACATGCCCTTTACACC | 341 | 62 | SEQ ID N. 40 |

### Fusions Validation Using Total RNA-Seq Analyses

Twelve samples were analyzed by RNA-seq. Libraries for RNA-seq were prepared with the TruSeq stranded mRNA kit (Illumina, San Diego, Calif., USA), as previously described[25]. The FASTQ files were processed to extract fusions. Alignment was performed with STAR v2.5.2b. STAR-Fusion v0.8.0 has been applied to detect fusion events.

### Copy Number Analyses

Genome-wide Copy Number Alterations (CNAs) was, in our laboratory, carried out on 34 patients. Experiments were performed by using Human Cytoscan HD (n=20) or SNP 6.0 arrays (n=14, Affymetrix, Santa Clara, Calif.). Data were processed by Affymetrix Genotyping Console to evaluate CEL files quality and then analyzed by Chromosome Analysis Suits software (version 4.0.0.385, Applied Biosystems by ThermoFisher). CNAs were filtered as follow: >1kb for CN loss and gains and probe count >8.

Moreover, CNAs of genes frequently amplified or deleted in ALL (e.g. IKZF1, PAX5, ETV6, JAK2, RB1, CDKN2A/B, BIG1, EBF1, ZFY, CRLF2, IL3RA, CSF2RA, P2RY8, SHOX) was performed on 41 cases using the SALSA MLPA (Multiplex Ligation-dependent Probe Amplification) P335 ALL-IKZF1 kit (MRC Holland, Amsterdam, the Netherlands). Coffalyser.Net software was used to analyze CNAs.

Identification of pH-Like Molecular Features

In order to identify possible Ph-like samples the inventors combine RNA-seq panel gene expression data, mutational screening of the three Ph-like associated mutated genes and fusion data[1,6].

The inventors performed an agglomerative clustering (ward linkage, euclidean distance) on the gene expression matrix for the genes identified in literature as possible signature for Ph-like samples[26]. Then, the inventors performed a principal component analysis (PCA) to visualize the group of suspected Ph-like. All the samples labeled as Ph-like show log FPKM for CRLF2 >5. Sanger sequencing primers and annealing temperatures for CRLF2, IL7 and JAK2 mutations are reported in Table 3.

### Fusion Pipeline

B-ALL fusion database creations: Using Medline/Pubmed, inventors revised literature on B-ALL from 2013 to 2019, to create a B-ALL fusion database to use in their pipeline for “literature retention filter”. They used four keywords combination: “fusion AND acute lymphoblastic leukemia”, “transcriptome AND acute lymphoblastic leukemia”, “genomic landscape AND acute lymphoblastic leukemia” and “genomic profiling AND acute lymphoblastic leukemia”. Additionally using Medline/Pubmed, literature on B-ALL from 2019 to 2021 was revised to create a B-ALL fusion database with “fusion AND acute lymphoblastic leukemia” keywords combination. A more of 730 fusion list described in B-ALL at different ages was obtained. Considering B-ALL subgroup annotations and described reciprocal fusions a list of 967 fusion transcripts was obtained.
raw sequencing data were converted to FASTQ file format
sample FASTQ files were analyzed combining 4 tools:
   FusionCatcher
   STAR-Fusion
   RNA-Seq Alignment
   TopHat Alignment

Manta (the RNA-Seq Alignment fusion caller) and STAR-Fusion take advantage of STAR aligner, while TopHat-Fusion is built to run on TopHat alignments, and FusionCatcher combines BLAT, STAR, Bowtie and Bowtie2[19-21]. The reference ‘Homo sapiens UCSC hg19’ (RefSeq and Gencode gene annotations) was used for all the aligners.
The 4 tool fusion outputs were combined in a list. This list will be elaborated:
   for each sample inventors determine how many times a fusion is called. To do this, further checks are needed:
   inventors consider different breakpoint of the same fusion as a single event for that sample
   inventors consider reciprocal fusion as a single event for that sample
   inventors check if a fusion gene partner is called with an alias
   inventors check if at the same breakpoint locus is present more than one gene (in case of a same breakpoint but a different gene name we checked on Ucsc the possible compresence of two genes in the same locus (https://genome.ucsc.edu))

These steps are simplified in the scheme 1 above.
inventors reject fusions presents in run controls. If fusion is described in B-ALL, fusion will be retained
inventors annotate fusions described in normal samples
inventors check if fusion described in normal samples are also already described in ALL (acute lymphoblastic leukemia). If fusion is described in B-ALL, fusion will be retained
inventors check if a fusion is frequently called in their samples
inventors retained fusions detected by at least three tools
inventors introduced two further criteria to retain or reject fusions detected by two or one tool.

In order of priority:
inventors firstly retained fusions (and their reciprocal transcripts) that were already reported in ALL literature and included in the list of known fusions created as described below.
All the other fusions identified by one single tool were discarded.
Second, for transcripts detected by two different tools, but not previously published in ALL, inventors check fusion marked “false positive” by Fusion Catcher (FusionCatcher summary candidate fusion output file)
Third, of the remaining fusions detected by two tools, inventors retained the ones labeled as significant for at least one of the three following events: a) Manta positive score, b) fusion positive comments in “FusionCatcher summary candidate fusion” output, c) EBF1 and ERG read-throughs (FusionCatcher).

Some post processing filtering steps are shown simplified in the following scheme 2:
In addition, the definitive filtered fusion list was further integrated with five public fusion database: a) https://www.tumorfusions.org/, b) https://cancer.sanger.ac.uk/cosmic/fusion, c) http://www.kobic.re.kr/chimerdb/chimerkb, d) https://mitelmandatabase.isb-cgc.org/mb_search, e) https://ccsm.uth.edu/FusionGDB/in order to annotate if each specific fusion was reported in other solid and hematological tumors (excluding ALL) and/or in normal samples.

### Results

### Example 1

### Fusion Transcripts are Common in Ph−/−/− B-ALL Cases and are Heterogeneously Detected by Diverse Mining-Fusion Tools

Ph−/−/−. Analysis of the sequencing data by four mining-fusion tools, led to the identification of 797 candidate fusions. In agreement with previous studies[16,17], the inventors found that fusion calling was heterogeneous across different tools: Manta (n=345)>STAR-Fusion (n=311)>FusionCatcher (n=99)>TopHat-Fusion (n=62). To reduce the number of false-positives, the inventors applied a stringent filtering process (FIG. 1A and methods section) by using the following criteria: 1) the inventors kept candidate fusions detected simultaneously by three or four tools in the same sample, 2) the inventors applied a customized pipeline on fusions detected only by one or two tools. Applying the above mentioned criteria, the inventors identified 65 bona fide fusion transcripts characterized by 108 different breakpoints, in 41 out of 63 samples (Table 1).

The majority of the fusions were detected by one (n=21) or two (n=22) tools while fifteen and seven fusions were detected by four and three tools, respectively (FIG. 1B, Table 4).

TABLE 4
Summary of fusions detected in the 41/63 samples of Ph—/—/— B-ALL patients
using four different tools of analyses with relative B-ALL and other tumor literature.

| | | | | | | | | | Adult B-ALL | | Normal (N), |
|---|---|---|---|---|---|---|---|---|---|---|---|
| | | | | | | | | Validation | Subgroup | | Solid & other |
| | | | | | | | | Methods | Literature | | Hematological |
| | | | | | | | Fusion | (Indirect | (Other Age | BALL | Malignancy |
| Pt N | Phase | Sample | FC | RSA | THA | SF | Summary | Validation) | Groups) | References | Literature |
| 1 | D P | 8 | ● | ● | ● | ● | THADA-CDH1 | FISH, (RNA- | NEW | | |
| | | | | | | | | Seq. Karyotype) | | | |
| | | | | | ● | | PAX5-ZCCHC7 | RT-PCR, (RNA- | PAX5, Ph-like | [13] | |
| | | | | | | | | Seq, SNP-A) | | | |
| | R P | 27 | ● | ● | ● | ● | TET3-ETV6 | FISH | NEW | | |
| | | | | | ● | | PAX5-ZCCHC7 | (SNP-A) | PAX5, Ph-like | [9, 13, 14] | |
| 2 | D | 9 | | | | ● | PAX5-ZCCHC7 | (SNP-A) | PAX5, Ph-like | [9, 13, 14] | |
| 3 | R P | 20 | | ● | ● | | ARHGAP26-NR3C1 | RT-PCR | NS | [16] | N[27] |
| | | | ● | | ● | | RUNX1-RCAN1^{R} | RT-PCR | NEW | | a: BIC, UCS; |
| | | | | | | | | | | | b: AC; |
| | | | | | | | | | | | e: CSCC, ECA |
| 5 | D | 12 | ● | ● | ● | ● | ABL2-RCSD1^{R} | | Ph-like | [10, 13, 14, | |
| | | | | | | | | | | 28, 29] | |
| 6 | D | 13 | ● | | ● | | P2RY8-CRLF2^{R} | | CRLF2, Ph-like, | [13, 14, 30, | |
| | | | | | | | | | PAX5 P80R, HeH, | 31] | |
| | | | | | | | | | NS | | |
| 9 | D | 17 | ● | | | | DUX4-IGH | (RNA-Seq) | DUX4r | [14, 32] | |
| 10 | D P | 18 | ● | | ● | | (ENSG00000231231) | (SNP-A, MLPA) | NEW | | |
| | | | | | | | LINC01423-ERG^{R} | | | | |
| | | | | | | ● | ZEB2-CXCR4 | RT-PCR | DUX4-r, NH- | [10] | |
| | | | | | | | | | HeH, NS, LH | | |
| | | | | | | | | | (AYA), NS | | |
| | | | | | | | | | (AYA) | | |
| | R P | 52 | ● | | ● | | (ENSG00000231231) | (SNP-A, MLPA) | NEW | | |
| | | | | | | | LINC01423-ERG^{R} | | | | |
| 11 | R | 19 | ● | ● | ● | ● | TCF3-ZNF384 | FISH, (RNA- | ZNF384 | [33] | |
| | | | | | | | | Seq, SNP-A) | | | |
| 12 | D | 21 | ● | ● | ● | ● | TAF15-ZNF384^{R} | RT-PCR.(RNA- | ZNF384 | [13, 14, 34, | |
| | | | | | | | | Seq, Karyotype) | | 35] | |
| 13 | R | 22 | ● | ● | ● | ● | ABL1-RCSD1^{R} | FISH | Ph-like | [10, 36] | |
| 14 | D | 23 | ● | | | | EBF1-LINC02227 | (SNP-A) | NEW | | |
| 16 | R | 25 | | | | ● | ZEB2-CXCR4 | | DUX4-r, NH- | [10] | |
| | | | | | | | | | HeH, NS, LH | | |
| | | | | | | | | | (AYA), NS | | |
| | | | | | | | | | (AYA) | | |
| 17 | D | 26 | ● | ● | ● | ● | TFG-GPR128 | (RNA-Seq) | Ph-like (Ped) | [31] | d: AML, AC, |
| | | | | | | | (ADGRG7)^{R} | | | | Acy, MM; |
| | | | | | | | | | | | N[27, 37] |
| | | | | ● | | ● | ARL11-RB1 | (RNA-Seq) | NEW | | |
| 18 | D | 28 | | ● | | ● | ARHGAP26-NR3C1 | RT-PCR | NS | [16] | N[27] |
| | | | | ● | | ● | UBXN4-CRCX4 | RT-PCR | NEW | | |
| 19 | D | 29 | ● | ● | ● | ● | KMT2A-MLLT1 | | MLLr | [5, 10, 13, | |
| | | | | | | | | | | 14] | |
| 22 | D | 32 | ● | ● | ● | ● | KMT2A-MLLT1 | (Karyotype) | MLLr | [5, 10, 13, | |
| | | | | | | | | | | 14] | |
| 23 | R P | 81 | | ● | ● | | ARID5B-KMT2D | | NEW | | |
| 24 | D | 34 | ● | ● | ● | | NONO-TFE3 | | NS | [9] | b, c, d: RC |
| 25 | D | 35 | | ● | ● | ● | ARHGAP26-NR3C1 | RT-PCR | NS | [16] | N[27] |
| | | | | ● | | ● | ZFPM1-SLC7A5 | | NEW | | |
| | | | ● | | | ● | CBFA2T3-SLC7A5^{R} | RT-PCR | Ph-like (Ped) | [31] | |
| | | | | | | ● | NCOR2-BCL7A | RT-PCR | HeH, MLLr, NS | [27] | |
| 26 | D | 36 | | ● | | ● | PAX5-ZCCHC7 | | PAX5, Ph-like, | [9, 13, 14] | |
| | | | | | | | | | NS | | |
| 27 | D | 37 | ● | ● | ● | ● | PAX5-ETV6 | | PAX5 Fusions | [13] | |
| | | | ● | ● | ● | ● | TFG-GPR128 | | Ph-like (Ped) | [31] | d: AML, AC, |
| | | | | | | | (ADGRG7)^{R} | | | | Acy, MM; |
| | | | | | | | | | | | N [27, 37] |
| 32 | D | 42 | ● | ● | ● | ● | ZNF384-EP300^{R} | | ZNF384 | [10, 13, 14, | |
| | | | | | | | | | | 32, 38] | |
| | | | | | | ● | CBFA2T3-SLC7A5 | RT-PCR | Ph-like (Ped) | [31] | |
| | | | | ● | | ● | PTMA-CXCR4 | | NEW | | |
| | | | | | | ● | OAZ1-DOT1L | | NS | [16] | e: OSC, N[39] |
| 33 | D | 43 | ● | ● | | ● | EP300-ZNF384 | | ZNF384 | [10, 13, 14, | |
| | | | | | | | | | | 32, 38] | |
| | | | | | | ● | ARHGAP26-NR3C1 | | NS | [16] | N[27] |
| 36 | D | 46 | ● | | ● | | PPP3CC-CCAR2 | RT-PCR, (SNP-A) | NEW | | e: OSC, HNSCC |
| 38 | R | 51 | ● | ● | ● | ● | PAG1-PLAG1^{R} | RT-PCR | NEW | | |
| | | | | | ● | | ARHGAP26-NR3C1 | (SNP-A) | NS | [16] | N[27] |
| 39 | D | 53 | ● | | | | IGH-MYC | (Karyotype) | BCL2/MYC | [13] r | d: BL, B-PLL, |
| | | | | | | | | | | | DLBCL, FL, HD, |
| | | | | | | | | | | | MCL, MM, PCL |
| | | | | ● | ● | ● | FLI1-ZBTB16^{R} | | NEW | | |
| | | | | ● | | ● | CRTC1-SUGP2 | | NEW | | |
| | | | | ● | | ● | ALDH3B1-TFDP1 | | NEW | | |
| | | | | ● | | | CPSF6-BRD1 | | NEW | | |
| | | | ● | ● | | ● | OAZ1-DOT1L | | NS | [16] | e: OSC, N [39] |
| | | | | | | ● | | | | | |
| 40 | R | 54 | ● | | ● | | MEF2D-BCL9 | | MEF2D | [10, 13, 14] | |
| 41 | D | 55 | | | | ● | CXCR4-GNAS | | NEW | | N[27] |
| | | | | | | | CYFIP2-EBF1 | RT-PCR | ETV6-RUNX1- | [13, 14] | |
| | | | | | | | | | like, iAMP21 | | |
| | | | | | | | | | (AYA) | | |
| 42 | D | 57 | ● | | ● | ● | IKZF1-IGKV5-2^{R} | RT-PCR | NEW | | |
| | | | ● | | | | P2RY8-IGH | | NEW | | d: MM |
| 43 | R | 58 | ● | ● | ● | ● | ZNF384-EP300^{R} | | ZNF384 | [10, 13, 14] | |
| 44 | D | 60 | | | | ● | NR3C1-ARHGAP26 | | NS | [16] | N[27] |
| | | | | ● | | ● | ZEB2-CXCR4 | RT-PCR | DUX4-r, NH- | [10] | |
| | | | | | | | | | HeH, NS, LH | | |
| | | | | | | | | | (AYA), NS | | |
| | | | | | | | | | (AYA) | | |
| 45 | D | 61 | ● | | ● | | (IGHJ5 & J6) | | BCL2/MYC | [13] | c, d: Lymphomas |
| | | | | | | | IGH-BCL6^{R} | | | | |
| 46 | D | 62 | ● | | | ● | ZEB2-CXCR4 | RT-PCR | DUX4-r, NH- | [10] | |
| | | | | | | ● | | | HeH, NS, LH | | |
| | | | | | | | | | (AYA), NS | | |
| | | | | | | | | | (AYA) | | |
| 47 | D | 63 | | | | | ZNF362-SMARCA4 | | ZNF384 | [14] | |
| 48 | D | 64 | ● | ● | ● | ● | ZNF384-TCF3^{R} | | ZNF384 | [33] | |
| | | | | | | ● | NCOR2-BCL7A | RT-PCR | HeH, MLLr, NS | [27] | |
| 49 | D | 65 | ● | ● | | ● | NUMA1-CSF1R | RT-PCR | NEW | | |
| 50 | R | 66 | | | | ● | ZEB2-CXCR4 | RT-PCR | DUX4-r, NH- | [10] | |
| | | | | | | | | | HeH, NS, LH | | |
| | | | | | | | | | (AYA), NS | | |
| | | | | | | | | | (AYA) | | |
| 51 | R2 | 68 | ● | ● | | | MARCH8-NCOA4 | RT-PCR | NEW | | |
| 55 | D | 86 | | ● | | | BCL7A-NCOR2 | | HeH, MLLr, NS | [27] | |

Pt: Patient, D: Diagnosis, R: Relapse, Pt: patients, RSA: RNA-Seq Alignment, THA: TopHat Alignment, FC: FusionCatcher and SF: STAR-Fusion, ^{R}: Reciprocal fusion, ( ): B-ALL Literature not referred to adult patients, Ped: Pediatric, AYA: Adolescents-Young Adults, NS: described in B-ALL but age subgroup not specified, NH-HeH: near-haploid ALL and high hyperdiploid, HeH: high hyperdiploid, LH: Low Hyperdiploid, N: Normal-Healthy donors, MM: Multiple Myeloma, BIC: Breast Invasive Carcinoma, UCS: Uterine Carcinosarcoma, AC: Adenocarcinoma, CSCC: Cervical Squamous Cell Carcinoma, ECA: Endocervical Adenocarcinoma, AML: Acute Myeloid Leukemia, ACy: Astrocytoma, RC: Renal Carcinoma, OSC: Ovarian Serous Cystadenocarcinoma, HNSCC: Head and Neck Squamous Cell Carcinoma, BL: Burkitt lymphoma, B-PLL: B cell prolymphocytic leukemia, CLL: Chronic lymphocytic leukemia, DLBCL: Diffuse large B-cell lymphoma, FL: Follicular lymphoma, HD: Hodgkin disease, MCL: Mantle cell lymphoma, PCL: Plasma cell leukemia. N: Normal samples, a: [40], b: [41], c: [42], d: [43], e: [44].

Moreover, discordant results among different tools were frequent for cryptic transcripts. For example, read-throughs (e.g. LINC01423-ERG) could be detected only by FusionCatcher. Similarly, FusionCatcher was the best tool to detect immunoglobulin gene fusions such as IGH-DUX4, IGH-P2RY8, IGH-MYC and IGH-BCL6. The latter fusion was detected also by TopHat-Fusion tool.

Next, the inventors validated our filtering strategy using different experimental approaches (RT-PCR, FISH, RNA-seq, CNAs or CBA). For this purpose, the inventors selected different candidate fusions based on the following criteria: i) fusions never reported, ii) fusions detected by only one or two tools, and iii) availability of sample's biological materials.

The inventors tested 13 novel fusion transcripts and 25 fusions that have been already reported in literature with an overall accuracy of validation of ˜97% (37/38 fusions experimentally validated, Table 4).

Overall, fusion transcripts were a common event in Ph−/−/− B-ALL, with 65.1% of samples (41/63) carrying at least one translocation, not identified by conventional diagnostics. Among them, 61% had only one detectable fusion, while 39% was characterized by multiple fusions. The inventors identified 50 fusions in 29 samples at diagnosis and 15 fusions in samples at relapse (n=12 cases). In term of chromosomal distribution, all chromosomes with the exception of chromosome 6, 15 and 18 were involved in fusion events. Twenty-four out of 65 fusion transcripts involved partner genes located on different chromosomes, while 41 fusion genes were intra-chromosomal and involved neighbor genes (e.g. UBXN4-CXCR4, PAX5-ZCCHC7 and TGF-GPR128). In addition, different genes involved in fusions had multiple partners and, in particular, IGH (MYC, DUX4, BCL6, and P2RY8), CXCR4 (ZEB2, UBXN4, PTMA, GNAS), ZNF384 (EP300, TCF3 and TAF15), PAX5 (TET3 and ZCCHC7), RCSD1 (ABL1 and ABL2), P2RY8 (CRLF2 and IGH) and ETV6 (PAX5 and TET3) (FIG. 1C).

### Example 2

Ph−/−/− B-ALL patients harbors different ALL-associated fusions: frequencies, cryptic fusions and occurrence in other tumors

Forty-three out of 65 fusions have been previously described in Ph− ALL cases (Table 4). They included rare fusions previously described in ALL samples (e.g. CBFA2T3-SLC7A5 and CYFIP2-EBF1, FIG. 6-7) and recurrent fusions belonging to two major classes of rearrangements with clinical relevance in ALL: ABL-class fusions and ZNF384 rearrangements. Patients carrying ABL-class fusions can be successfully treated with tyrosine kinase inhibitors [45] while the outcome of cases carrying ZNF384 rearrangements is largely dependent on the partner gene [8]. Based on the availability of biological material of these two subtypes, the fusions TCF3-ZNF384, TAF15-ZNF384 and RCSD1-ABL1 were selected for further cytogenetic and/or molecular analyses.

At diagnosis, the patient #11 showed a normal karyotype that progressed to 45,XY,−7[9]/45,XY,−7,del(9)(p11)[3]/46,XY[12] at relapse. Relapse sample FISH analysis with specific BAC probes for ZNF384 and TCF3 genes confirmed the presence of TCF3-ZNF384 fusion gene on derivative chromosome 19 associated with a 3′ deletion of TCF3 in 98% of nuclei and in 20 metaphases (FIG. 8A-B). The rearrangement was caused by t(12,19)(p13,p13) which remained cryptic by CBA. SNPs array analysis showed a heterozygous loss of 3′ TCF3 and a gain of 3′ ZNF384 (FIG. 8C). These results were in line with the observation from FISH analysis. The integration of SNPs and FISH results suggested that TCF3-ZNF384 rearrangement may result from an unbalanced translocation between chromosome 12 and chromosome 19 leading to partial trisomy for 12p13-pter and monosomy for 19p13-pter.

The RCSD1-ABL1 fusion was detected in the sample 22 (patient #13) with normal karyotype. These fusions were associated with t(1,9)(q24,q34) that are not usually cryptic by CBA. However, due to the low number of viable cells, the inventors were not able to detect the chromosome translocation using CBA analysis. FISH analysis confirmed the presence of RCSD1-ABL1 and ABL1-RCSD1 fusion genes in 4% of analyzed nuclei (FIG. 8D). STAR-Fusion and Manta revealed the reciprocal fusion ABL1-RCSD1 and probable two alternative splicing events between RCSD1 exon 2 or 3 and ABL1 exon 4 (FIG. 8E). To confirm the presence of TAF15-ZNF384 in the sample 21 (46,XX,t(12,17)(p13,q21)[8]/46,XX[12]), the inventors performed Sanger sequencing analysis and showed that the fusion occurs conjoining the end of TAF15 exon 6 to the beginning of ZNF384 exon 3 preserving exons reading frame (FIG. 8F-G).

Lastly, 10 of the detected fusions were previously described in other solid and onco-hematologic tumors (e.g. NONO-TFE3 [46], IGH-MYC[44]) (Table 4). The most recurrent ones were ARHGAP26-NR3C1 (9.2%, 6/65), ZEB2-CXCR4 (7.7%, 5/65), PAX5-ZCCHC7 (6.1%, 4/65), BCL7A-NCOR2 (4.6%, 3/65)(FIG. 9-12) and EP300-ZNF384 (4.6%, 3/65). ARHGAP26-NR3C1, OAZI-DOTIL and TGF-GPR128 were described both in B-ALL and in normal samples [16,17,37,39], while CXCR4-GNAS, were previously described only in normal samples [27]. ARHGAP26-NR3C1 in sample #51 seems to derive from a deletion of one allele (FIG. 9), ZEB2-CXCR4 [10], that was described in several Ph negative subgroups, presented in our positive samples, two in-frame breakpoints (ZEB2 ex2 or ex1-CXCR4 ex2) (FIG. 10). PAX5-ZCCHC7 fusion, described in Ph-like and PAX5 subgroups, is characterized in patients #1 and #2 by heterozygous and homozygous deletions, notably patient #1 preserved the same breakpoint at diagnosis and relapse (FIG. 11).

### Example 3

### Novel Fusion Transcripts in Ph−/−/− B-ALL

Twenty-two out of 65 fusions have never been reported in B-ALL cases. To rule out Ph-like positive cases, the inventors applied the gene signature described by Chiaretti and colleagues to our gene expression data [26] and identified eight samples with molecular features associated with Ph-like subgroups[6], such as: i) CRLF2 over-expression, ii) P2RY8-CRLF2/IGH, PAX5-ZCCHC7 fusions, iii) mutations in JAK2, CRLF2 and IL7 genes. Based on this analysis, in five patients the inventors identified new fusions that may be associated with the Ph-like phenotype: THADA-CDH1, TET3-ETV6, NUMA1-CSF1R, IKZF1-IGKV5-2 and EBF1-LINC02227. An additional borderline sample (ID 20) had a CRLF2 mutation and RUNX1-RCAN1 (FIG. 2A). To our knowledge, the translocation between THADA and CDH1 genes has never been reported before. The inventors identified one positive case (patient #1) at diagnosis characterized by the following karyotype: 46,XY,t(2,16)(p15,q22)[5]/46,XY[15]. FISH analysis with specific BAC clones for THADA and CDH1 genes showed the presence of the THADA-CDH1 rearrangement associated with a partial THADA deletion (FIG. 2B-C) as confirmed by SNPs array analysis (data not shown). In the translocation, the breakpoint occurs at the end of the exon 36 (NM_022065, chr2: 43506903, −). The biological function of THADA is still unclear, but an association with TRAIL-induced apoptosis has been suggested[47]. CDH1 is located on chromosome 16q22.1 and comprises 16 exons. The breakpoint on CDH1 occurred in exon 3 (NM_004360, chr16: 68835572, +) (FIG. 2D). E-cadherin, the protein coded from CDH1 gene, is a tumor suppressor involved in inhibition of β-catenin from the binding to different transcription factors [48]. The fusion was not detected at relapse. However, the inventors identified the novel fusion TET3-ETV6 in the same patient at relapse. This was confirmed by FISH analysis with positive signal pattern combining LSI ETV6-RUNX1 and RP11-980B20. TET3-ETV6 and its reciprocal fusion were identified in 25% of clones (FIG. 2E, Table 4). The breakpoint occurred at the end of exon 1 (NM_001287491, chr2: 74213833, +). TET3 is involved in the oxidation of the 5-methylcytosine (5mC) to 5-hydroxymethylcytosine (5hmC) that promotes gene transcription [49-51]. The breakpoint led to the loss of the entire exon 1 (NM_001987, chr12: 11905384, +) of the transcriptional repressor ETV6 (FIG. 2F) [52,53].

Several rearrangements of CSF1R fusions have been described in B-ALL Ph-like [31]. The inventors identified a new in-frame fusion involving NUMA1 and CSF1R genes in a normal karyotype sample. In this fusion, the breakpoint occurred at the end of NUMA1 exon 26 and at the beginning of CSF1R exon 12 (NUMA1:NM_006185, chr11:71714933 −, CSF1R:NM_005211, chr5:149441412, −, FIG. 3A). The rearrangement preserved NUMA1 domains and CSF1R ATP binding domain and protein tyrosine kinase catalytic domain, while CSF1R Ig, Ig-like and dimerization domains were lost. Read depth analysis showed a very low expression of exons upstream the junction and a clear upregulation of exons downstream breakpoint of CSF1R (FIG. 3B-C). In the sample 57, CRLF2 mutated, the inventors identified the in frame fusion IKZF1-IGKV5-2 (IKZF1:NM_006060, chr7:50459561, +, IGKV5-2: ENST00000390244, chr2: 89197005, +), whose breakpoint mapped in exon 7 of IKZF1 and exon 2 of IGKV5-2. In the fusion all IKZF1 functional domains were preserved (FIG. 3D-E). The inventors detected a new EBF1 fusion that involved the neighbor long intergenic non-protein coding RNA LINC02227 in sample #23, also CRLF2 mutated. This fusion had three breakpoints that in all cases lead to EBF1 exon 4 and LINC02227 exon 2 rearrangement (FIG. 3F). The lymphoid transcription factor EBF1 is described to be deleted or partially deleted in B-ALL, with an enrichment in Ph-like cases[54]. Copy number data showed a heterozygous deletion between the two neighbor genes, suggesting that this fusion may result from a deletion event (FIG. 3G). Finally, the inventors identified, among Ph−/−/− B-ALL samples, several additional novel fusion transcripts, such as: PPP3CC-CCAR2 (FIG. 13), LINC01423-ERG (FIG. 14), RUNX1-RCAN1 (FIG. 15), PAG1-PLAG1 (FIG. 16), MARCH8-NCOA4 (FIG. 17) and UBXN4-CRCX4 (FIG. 18). A careful review of the literature highlighted that some of the above mentioned fusions such as PPP3CC-CCAR2[44] or RUNX1-RCAN1 [55], have been already reported in other tumors.

### Example 4

### The Translocations Lead to Altered Expression of Fusion Partner Genes

Finally, the inventors evaluated the expression of genes involved in each fusion (FIG. 23a-b), by comparing the relative transcript level of a gene of interest (e.g. MYC) involved in a fusion (e.g. IGH-MYC fusion) with its relative expression in samples not harboring any fusion involving that gene.

The inventors found that 20.2% of samples had a statistically significant variation in the expression of partner genes involved in the fusion (FIG. 19).

For example, RCSD1-ABL2 determined an over-expression of ABL2 (p<0.0005), while ARID5B-KTM2D caused a down-regulation of KTM2D (p<0.0005, FIG. 20A-B). In addition, some of the ALL crucial genes have been found upregulated when involved in fusion transcripts, such as MYC (sample 53), ZNF384(samples 19 and 64), ETV6(sample 27) and BCL6 (sample 61)(FIGS. 20C and 23). Interestingly, these genes showed a breakpoint dependent up/down-regulation, due to the fact that they are not expressed before (5′ gene) and downstream (3′ gene) the junction (FIG. 20 A-C). Breakpoint dependent regulation seems to affect other genes but in these cases expression variation need to be calculated only on the gene portion affected by the fusion and not the entire transcript (FIG. 20D).

Regarding the TGF-GPR128 fusion, that has been previously described in B-ALL and in normal samples[16,17,27], GPR128 is expressed neither in healthy donor samples and nor in not-rearranged Ph−/−/− cases, while it is upregulated only in our fused cases (FIG. 21A, FIG. 23). Finally, among cases with the recurrent ZEB2-CXCR4 fusion sample 18 showed the highest CXCR4 expression (p<0.05, FIG. 21B, FIG. 23).

### Discussion

Several studies are currently optimizing the characterization and risk stratification of B-Other ALL patients. Whole transcriptomic-based studies have successfully identified the Ph-like subgroup out of B-Other ALL patients improving the therapeutic options for these patients[6,56].

Here the inventors applied an RNA-sequencing panel approach coupled to an integrated pipeline to efficiently identify bonafide fusion transcripts in Ph−/−/− B-ALL samples. Combining data from four bioinformatics tools (STAR-Fusion, Manta, FusionCatcher and TopHat-Fusion), the inventors identified 65 candidate fusions in 63 Ph−/−/− B-ALL samples.

From a methodological point of view, the inventors observed a high level of heterogeneity in fusion calling using different mining-tools. The inventors demonstrated that using one single tool significantly under-estimated the number of detectable fusions in Ph−/−/− B-ALL patients, whereas the combination of four tools increased the robustness of fusion selection and the need of a fine filtering system.

The multi-tool approach was crucial for the identification of fusions affecting IG-genes that are frequently missed by most common pipelines.

Thanks to our strategy, the inventors selected 8.1% of the 797 candidate fusions obtained as four tool output.

Notably, a) the inventors would have lost 43 candidate fusions if the inventors had considered only those detected by 3 or 4 tools, and b) integration of a “literature filter” allowed us to retain 28 fusions (18/18 validated) that would have been discarded due to upstream pipeline processes. This strategy permitted us to retain fusions important for B-ALL classification (e.g. DUX4-IGH, ZNF362-SMARCA4).

The strength of our approach was supported by the high validation rate (97%), confirming that integration of multiple tools and filtering curation based on literature are needed to identify true fusions and to avoid false positives.

Interestingly, the inventors found that fusions are a common event in Ph−/−/− B-ALL (65.1% of cases) affecting almost all chromosomes. Moreover, multiple breakpoints characterize each fusion. Finally, the overall number of fusions did not differ between diagnosis and relapse.

However, few cases (#1, #3, #23, #10 and #51) showed diagnosis- or relapse-specific fusions. Patient #1 expressed PAX5-ZCCHC7 fusion at both diagnosis and relapse, the chimera THADA1-CDH1 was expressed only at diagnosis (patient #1), while TET3-ETV6 were detected only at relapse (patient #1). The persistence of this fusion was confirmed by FISH analysis in 16% of analyzed 27.6% recipient cells in the sample collected after the following salvage chemotherapy (without achieving a complete remission). In this case, multiple factors, including high risk clinical features, poor therapeutic response, identification of JAK2 mutations, CRLF2 upregulation and PAX5-ZCCHC7 fusion at both diagnosis and relapse, suggest a Ph-like phenotype, as confirmed by our analysis[6]. The inventors detected fusion genes in patients #3 and #23 only at relapse, while patient #10 expressed LINC01423-ERG at both time points, losing ZEB2-CXCR4 expression at disease progression. On the other hand, patient #51 acquired the new fusion MARCH8-NCOA4 at the second relapse. These results suggest, that our integrated approach may be a powerful strategy to detect known and unknown fusions and monitor their dynamics across disease evolution in Ph−/−/− B-ALL patients.

The inventors then characterized the nature of fusion breakpoints [57] and showed that Ph−/−/− fusions arise from different mechanisms: i) reciprocal fusions (e.g. ABL1/2-RCSD1, RUNX1-RCAN1 and PAG1-PLAG1), ii) non reciprocal fusions (e.g. CYFIP2-EBF1, UBXN4-CRCX4 and NCOR2-BCL7A), iii) deletions (PAX5-ZCCHC7, ARHGAP26-NR3C1, EBF1-LINC02227, LINC01423-ERG and PPP3CC-CCAR2). In only 4 fused cases with available karyotype (30/41) chromosomal rearrangements are already identifiable by karyotype analysis (IGH-MYC, THADA-CDH1, TAF15-ZNF384 and KMT2A-MLLT1).

From a biological point of view, the inventors identified 43 fusions already associated with ALL (e.g. ZNF384 rearranged (r) and Ph-like ALL, FIG. 4A) and 22 novel fusion transcripts. Based on the identified fusion transcripts, literature review and analysis for Ph-like determination, the inventors classify 51.2% of fused Ph−/−/− B-ALL into separate entities: a) ZNF384r in 18.5% (8/41), b) Ph-like in 17.1% (7/41), c) MLLr and BCL2/MYC both in 4.9% (2/41), d) MEF2Dr and DUX4r both in 2.4% (1/41) (FIG. 4A-B). Almost one third of Ph−/−/− cases (34.9%) was negative for fusion transcript detection and it could not be ascribed to any subtypes using our approach. Genes affected by fusions were grouped according to their biological function in the following processes: i) transcriptional regulation (40.3%), ii) epigenetics (33.3%), iii) signaling (5.6%), iv) cell cycle/apoptosis (4.2%), v) PI3K-AKT pathway (1.4%), vi) JAK-STAT pathway (1.4%), vii) other pathways/functions (9.7%) (FIG. 4C).

The inventors then evaluated the relative level of expression of genes involved in the fusions. Interestingly, the inventors found that almost 20% of cases showed a significant variation in the expression of genes involved in the fusions, with many fusion gene partners showing a breakpoint-dependent up or down regulation. According to our data, the evaluation of expression from both total gene and rearranged gene segment is important to provide an accurate analysis. This allows the definition of differentially expressed domains that may be potentially targeted, an event that could be underestimated by limiting the analyses on the full-length transcript.

For a therapeutic purpose, based on the current literature the inventors identified available pharmacological inhibitors of genes for 30 out of 43 gene fusions. Some of these inhibitors have been experimentally tested in ALL samples and are currently known for their efficacy such as TKi against ABL-class fusions, while other fusions have been predicted as potential targets of commercially available inhibitors (FIG. 5, Table 5).

TABLE 5
Summary of the known inhibitors against
partners genes involved in each fusion.

| FUSIONS: | | |
|---|---|---|
| GENE A + | | |
| GENE B | GENE A INHIBITOR | GENE B INHIBITOR |
| PAX5-ZCCHC7 | NA | NA |
| THADA-CDH1 | NA | NA |
| RCSD1-ABL2 | NA | TKi [58, 59] |
| CRLF2-P2RY8 | PI3K/mTORi, JAKi, | NA |
| | TKi [31, 59-61] | |
| DUX4-IGH | DUX4i [62] | NA |
| ERG-LINC01423 | Peptidomimetics | NA |
| | Anti-ERG [63] | |
| ZEB2-CXCR4 | NA | Plerixafor, |
| | | AMD070, BL-8040, |
| | | BMS-936564 [64-70] |
| TCF3-ZNF384 | NA | NA |
| ARHGAP26-NR3C1 | NA | Glucocorticoid |
| | | receptor |
| | | inhibitor [71] |
| RUNX1-RCAN1 | NA | Dipyridamole [72] |
| TAF15-ZNF384 | α-amanitin [73] | HDACi [74] |
| RCSD1-ABL1 | NA | TKi [75, 76] |
| EBF1-LINC02227 | NA | NA |
| ARL11-RB1 | NA | NA |
| TFG-GPR128 | NA | NA |
| TET3-ETV6 | NA | NA |
| ZNF384-EP300 | HDACi [74] | NA |
| UBXN4-CXCR4 | NA | Plerixafor, |
| | | AMD070, BL-8040, |
| | | BMS-936564 [64-70] |
| KMT2A-MLLT1 | DOT1Li [7.77-79] | NA |
| NONO-TFE3 | NA | PI3K/mTORi [80] |
| CBFA2T3-SLC7A5 | Dimethylfasudil [81] | JPH203 [82] |
| NCOR2-BCL7A | HDACi [83] | NA |
| ZFPMI-SLC7A5 | NA | JPH203 [82] |
| PAX5-ETV6 | NA | NA |
| OAZ1-DOT1L | NA | DOT1Li [7.77-79] |
| PTMA-CXCR4 | JNKi, ERKi, PI3Ki | Plerixafor, |
| | [84, 85] | AMD070, BL-8040, |
| | | BMS-936564 [64-70] |
| PPP3CC-CCAR2 | Cyclosporine | NA |
| | A, Tacrolimus [86] | |
| PAG1-PLAG1 | NA | NA |
| ALDH3B1-TFDP1 | Aldehyde | NA |
| | Dehydrogenase i [87] | |
| CPSF6-BRD1 | NA | BETi [88] |
| CRTC1-SUGP2 | NA | NA |
| FLI1-ZBTB16 | TK216 [89] | NA |
| IGH-MYC | NA | MYCi361, |
| | | BETi [90, 91] |
| MEF2D-BCL9 | HDACi [10] | NA |
| CXCR4-GNAS | Plerixafor, | PI3K/AKTi [92] |
| | AMD070, BL-8040, | |
| | BMS-936564 [64-70] | |
| CYFIP2-EBF1 | NA | NA |
| IKZF1-IGKV5-2 | NA | NA |
| P2RY8-IGH | NA | NA |
| IGH-BCL6 | NA | BCL6i [93] |
| ZNF362-SMARCA4 | JAKi [14] | NA |
| NUMA1-CSF1R | NA | CSF1Ri, TKi [31, 59] |
| MARCH8-NCOA4 | AKTi [94] | NA |
| ARID5B-KMT2D | NA | PI3K/mTORi [95] |

In the table NA = not available inhibitors, i = inhibitor.
JAK = janus kinase, TK = tyrosine kinase, HDAC = histone deacetylase, BET = bromodomain and extra-terminal motif, ERK = extracellular signal-regulated kinase, PI3K = Phosphatidylinositol-4,5-Bisphosphate 3-Kinase, mTOR = mechanistic target of rapamycin kinase, CSF1R = colony stimulating factor 1 receptor, DOTL1 = DOT1 like histone lysine methyltransferase, DUX4 = Double Homeobox 4.

The present invention allows the identification of fusion transcripts not detectable by conventional methodologies, can improve the characterization of one third of Ph−/−/− B-ALL cases. One third of the identified fusions has never been reported in ALL patients before. Although the pathogenic role of the identified fusions needs functional studies, the use of an NGS-based RNA approach with a powerful multi-level data analysis could be useful for a better classification, for disease monitoring and in some cases, therapeutic decisions (e.g. ABL1-2/RCSD1, MLLr, NUMA1-CSF1R), that may improve the outcome of Ph−/−/− B-ALL patients.

### Example 5

Application of the Method of the Invention to Other b-all Subtypes, Other Hematological and Solid Tumors: Preliminary Data.

To understand if our approach could be applied to other tumors we sequenced and analysed further 115 samples:
B-ALL (n=62)
3 t(1,19)
1 t(4,11)
22 t(9,22)/Ph+
36 Ph−/−/−

### Other Haematological Tumors:

T-ALL (n=10)
B-Cell Lymphoblastic Lymphoma (n=3)
T-Cell Lymphoblastic Lymphoma (n=2)
High grade Lymphoma (Double Hit, n=1)
Lympho/myeloid acute leukemia (n=2)
Mieloid leukemias (n=8)
5 Acute myeloid leukemia
1 Essential thrombocythemia
1 Myelodysplastic syndrome
1 Hypereosinophilic syndrome

### Solid Tumors:

17 esophageal carcinoma
4 sarcomas
1 lynch syndrome
1 skin carcinoma
1 breast cancer
1 FFPE fusion control sample

In these samples we experimentally validated with different methodologies [e.g. RT-PCR and Fluorescent In Situ Hybridization (FISH), Karyotype] further 46 fusions. Some of these rearrangements were never described before.

Sanger sequencing further primers and annealing temperatures rearrangement validations are reported in the following Table 6.

TABLE 6
Further primer sequences for RT-PCR and for Sanger sequencing along
with amplicon size (bp) and annealing temperature (T °C.)

| | | Annealing | | | |
|---|---|---|---|---|---|
| | | T° | Size | | SEQ |
| Target gene | Primers sequences (5′-3′) | (C) | (bp) | RefSeq | ID N. |
| RAB3IP ex3 F | ACGAAGCCCATCTGTTTTGG | 57.5 | 154 | NM_175623 | 41 |
| HMGA2 ex5 R | GTCCTCTTCGGCAGACTCTT | | 154 | NM_003483 | 42 |
| MSI2 ex7-8 F | ACTACCAACAGGCACAGAGG | 58.5 | 176 | NM_138962 | 43 |
| C17orf64 ex6 R | CTCTGGAGTTTCTGGGGCTT | | 176 | NM_181707 | 44 |
| TCF3 ex16 F | CCTCATGCACAACCACGC | 61 | 239 | NM_003200 | 45 |
| HLF ex4 R | CTCCTTCCTCAAGTCAGCCA | | 239 | NM_002126 | 46 |
| PLCL1 ex1 F | GATGAGGGACCGTCGCAG | 57.8 | 237 | NM_006226 | 47 |
| CDK1 ex3 R | CAACTCCATAGGTACCTTCTCCA | | 237 | NM_001786 | 48 |
| BANP ex6 F | GGACTACCTCTTCCACCGC | 62 | 180 | NM_079837 | 49 |
| SLC7A5 ex3R | AATGCCAGCACAATGTTCCC | | 180 | NM_003486 | 50 |
| ETV6 ex1 F | CCGGGAGAGATGCTGGAAG | 61.5 | 232 | NM_001987 | 51 |
| RP11-434C1.2 ex2 R | AGCTAGATTGGTTCCTGGTGA | | 232 | ENST00000536 | 52 |
| | | | | 492.1 | |
| ETV6 ex1 F | CCGGGAGAGATGCTGGAAG | 62 | 237 | NM_001987 | 53 |
| CCND2 ex5 R | TTGGTCCTGACGGTACTGC | | 237 | NM_001759 | 54 |
| CCND2 ex4 F | TGTACCCACCGTCGATGATC | 62 | 207 | NM_001759 | 55 |
| ETV6 ex2 R | GAACATGAAGTGGCGTCGAG | | 207 | NM_001987 | 56 |
| BCR ex1 F | GAACTCGCAACAGTCCTTCG | 62 | 236 | NM_004327 | 57 |
| MAPK1 ex2 R | TAGGTCTGGTGCTCAAAGGG | | 236 | NM_002745 | 58 |
| CD74 ex6 F | GATGCACCATTGGCTCCTG | 61.5 | 150 | NM_00102515 | 59 |
| | | | | 9 | |
| CAMK2A ex2 R | TGTGTTGATGATCTTGGCAGC | | 150 | NM_015981 | 60 |
| PAX5 ex6 F | CGGGGAGACTTGTTCACACA | 61.5 | 227 | NM_016734 | 61 |
| MLLT3/AF9 ex2 R | ATGTTACTGTGCTCCGGACC | | 227 | NM_004529 | 62 |

### LIST OF ABBREVIATION

B-ALL=B-cell acute lymphoblastic leukemia, Ph−/−/− B-ALL=Philadelphia Triple-Negative B-cell acute lymphoblastic leukemia, TKi=tyrosine kinase inhibitors, Ph+=Philadelphia positive, Ph-=Philadelphia negative, RT-PCR=Reverse Transcriptase-Polymerase Chain Reaction, FISH=Fluorescence In Situ Hybridization, RNA-seq=RNA sequencing, CNA=Copy number alteration, CN=copy number, CBA=Chromosome Binding Analysis, T=translocation, R=rearranged.

### REFERENCES

1. Iacobucci I, Mullighan C G. Genetic basis of acute lymphoblastic leukemia. J Clin Oncol. 2017; 35:975-83.
2. Hefazi M, Litzow M R. Recent advances in the biology and treatment of B-cell acute lymphoblastic leukemia. Blood Lymphat Cancer Targets Ther. 2018; Volume 8:47-61.
3. Sas V, Moisoiu V, Teodorescu P, Tranca S, Pop L, Iluta S, et al. Approach to the Adult Acute Lymphoblastic Leukemia Patient. J Clin Med. 2019; 8:1175.
4. Roberts K G, Gu Z, Payne-Turner D, McCastlain K, Harvey R C, Chen I-M, et al. High Frequency and Poor Outcome of Philadelphia Chromosome-Like Acute Lymphoblastic Leukemia in Adults. J Clin Oncol [Internet]. 2016; JC02016690073. Available from: http://www.ncbi.nlm.nih.gov/pubmed/27870571
5. Meyer C, Burmeister T, Groger D, Tsaur G, Fechina L, Renneville A, et al. The MLL recombinome of acute leukemias in 2017. Leukemia. 2018; 32:273-84.
6. Jain N, Roberts K G, Jabbour E, Patel K, Eterovic A K, Chen K, et al. Ph-like acute lymphoblastic leukemia: A high-risk subtype in adults. Blood. 2017;
7. Lilljebjorn H, Fioretos T. New oncogenic subtypes in pediatric B-cell precursor acute lymphoblastic leukemia. Blood. 2017.
8. Hirabayashi S, Ohki K, Nakabayashi K, Ichikawa H, Momozawa Y, Okamura K, et al. ZNF384-related fusion genes define a subgroup of childhood B-cell precursor acute lymphoblastic leukemia with a characteristic immunotype. Haematologica. 2017;
9. Lilljebjorn H, Henningsson R, Hyrenius-Wittsten A, Olsson L, Orsmark-Pietras C, Von Palffy S, et al. Identification of ETV6-RUNX1-like and DUX4-rearranged subtypes in paediatric B-cell precursor acute lymphoblastic leukaemia. Nat Commun. 2016;7.
10. Gu Z, Churchman M, Roberts K, Li Y, Liu Y, Harvey R C, et al. Genomic analyses identify recurrent MEF2D fusions in acute lymphoblastic leukaemia. Nat Commun [Internet]. 2016; 7:13331. Available from: http://www.nature.com/doifinder/10.1038/ncomms13331%5Cnhttp://www.ncbi.nlm.nih.gov/pub med/27824051%5Cnhttp://www.pubmedcentral.nih.gov/articlerender.fcgi?artid=PMC5105166
11. Zhang J, McCastlain K, Yoshihara H, Xu B, Chang Y, Churchman M L, et al. Deregulation of DUX4 and ERG in acute lymphoblastic leukemia. Nat Genet [Internet]. 2016; 48:1481-9. Available from: http://www.nature.com/doifinder/10.1038/ng.3691%5Cnhttp://www.ncbi.nlm.nih.gov/pubmed/27776115
12. Iacobucci I, Li Y, Roberts K G, Dobson S M, Kim J C, Payne-Turner D, et al. Truncating Erythropoietin Receptor Rearrangements in Acute Lymphoblastic Leukemia. Cancer Cell [Internet]. Elsevier Inc.; 2016; 29:186-200. Available from: http://dx.doi.org/10.1016/j.ccell.2015.12.013
13. Gu Z, Churchman M L, Roberts K G, Moore I, Zhou X, Nakitandwe J, et al. PAX5-driven subtypes of B-progenitor acute lymphoblastic leukemia. Nat Genet. 2019; 51:296-307.
14. Li J-F, Dai Y-T, Lilljebjorn H, Shen S-H, Cui B-W, Bai L, et al. Transcriptional landscape of B cell precursor acute lymphoblastic leukemia based on an international study of 1,223 cases. Proc Natl Acad Sci [Internet]. 2018;115:E11711-20. Available from: http://www.pnas.org/lookup/doi/10.1073/pnas.1814397115
15. Moorman A V. New and emerging prognostic and predictive genetic biomarkers in B-cell precursor acute lymphoblastic leukemia. Haematologica. 2016; 101:407-16.
16. Grioni A, Fazio G, Rigamonti S, Bystry V, Daniele G, Dostalova Z, et al. A Simple RNA Target Capture NGS Strategy for Fusion Genes Assessment in the Diagnostics of Pediatric B-cell Acute Lymphoblastic Leukemia. HemaSphere. 2019;
17. López-Nieva P, Fernández-Navarro P, Graña-Castro O, Andrés-León E, Santos J, Villa-Morales M, et al. Detection of novel fusion-transcripts by RNA-Seq in T-cell lymphoblastic lymphoma. Sci Rep. 2019;
18. Nicorici D, Satalan M, Edgren H, Kangaspeska S, Murumagi A, Kallioniemi O, et al.

FusionCatcher—a tool for finding somatic fusion genes in paired-end RNA-sequencing data [Internet]. bioRxiv. 2014. Available from: http://biorxiv.org/lookup/doi/10.1101/011650
19. Haas B J, Dobin A, Li B, Stransky N, Pochet N, Regev A. Accuracy assessment of fusion transcript detection via read-mapping and de novo fusion transcript assembly-based methods. Genome Biol. 2019;
20. Chen X, Schulz-Trieglaff O, Shaw R, Barnes B, Schlesinger F, Killberg M, et al. Manta: Rapid detection of structural variants and indels for germline and cancer sequencing applications. Bioinformatics. 2016;
21. Kim D, Salzberg S L. TopHat-Fusion: An algorithm for discovery of novel fusion transcripts. Genome Biol. 2011;
22. Roberts A, Trapnell C, Donaghey J, Rinn J L, Pachter L. Improving RNA-Seq expression estimates by correcting for fragment bias. Genome Biol. 2011;
23. Trapnell C, Hendrickson DG, Sauvageau M, Goff L, Rinn J L, Pachter L. Differential analysis of gene regulation at transcript resolution with RNA-seq. Nat Biotechnol. 2013;
24. Simonetti G, Padella A, Faria I, Fontana M C, Fonzi E, Bruno S. Aneuploid Acute Myeloid Leukemia Exhibits a Signature of Genomic Alterations in the Cell Cycle and Protein Degradation Machinery. 2019;
25. Padella A, Simonetti G, Paciello G, Giotopoulos G, Baldazzi C, Righi S, et al. Novel and rare fusion transcripts involving transcription factors and tumor suppressor genes in acute myeloid leukemia. Cancers (Basel). 2019;
26. Chiaretti S, Messina M, Grammatico S, Piciocchi A, Fedullo A L, Di Giacomo F, et al. Rapid identification of BCR/ABL1-like acute lymphoblastic leukaemia patients using a predictive statistical model based on quantitative real time-polymerase chain reaction: clinical, prognostic and therapeutic implications. Br J Haematol. 2018; 181:642-52.
27. Marincevic-Zuniga Y, Dahlberg J, Nilsson S, Raine A, Nystedt S, Lindqvist C M, et al. Transcriptome sequencing in pediatric acute lymphoblastic leukemia identifies fusion genes associated with distinct DNA methylation profiles. J Hematol Oncol. 2017;
28. Raca G, Gurbuxani S, Zhang Z, Li Z, Sukhanova M, McNeer J, et al. RCSD1-ABL2 fusion resulting from a complex chromosomal rearrangement in high-risk B-cell acute lymphoblastic leukemia. Leuk. Lymphoma. 2015.
29. Boer J M, Steeghs E M P, Marchante J R M, Boeree A, Beaudoin J J, Beverloo H B, et al. Tyrosine kinase fusion genes in pediatric BCR-ABL1-like acute lymphoblastic leukemia. Oncotarget. 2017;
30. Herold T, Schneider S, Metzeler K H, Hartmann L, Roberts K G, Konstandin N P, et al. Adults with Philadelphia chromosome-like acute lymphoblastic leukemia frequently have IGH-CRLF2 and JAK2 mutations, persistence of minimal residual disease and poor prognosis. Haematologica. 2017; 102:130-8.
31. Roberts K G, Li Y, Payne-Turner D, Harvey R C, Yang Y L, Pei D, et al. Targetable kinase-activating lesions in Ph-like acute lymphoblastic leukemia. N Engl J Med. 2014;
32. Schroeder M P, Bastian L, Eckert C, Gokbuget N, James A R, Tanchez J O, et al. Integrated analysis of relapsed B-cell precursor Acute Lymphoblastic Leukemia identifies subtype-specific cytokine and metabolic signatures. Sci Rep. 2019; 9:4188.
33. Liu Y F, Wang B Y, Zhang W N, Huang J Y, Li B S, Zhang M, et al. Genomic Profiling of Adult and Pediatric B-cell Acute Lymphoblastic Leukemia. EBioMedicine. 2016; 8:173-83.
34. Georgakopoulos N, Diamantopoulos P, Micci F, Giannakopoulou N, Zervakis K, Dimitrakopoulou A, et al. An adult patient with early Pre-B acute lymphoblastic leukemia with t(12;17)(p13;q21)/ZNF384-TAF15. In Vivo (Brooklyn). 2018;
35. Grammatico S, Vitale A, La Starza R, Gorello P, Angelosanto N, Negulici A D, et al. Lineage switch from pro-B acute lymphoid leukemia to acute myeloid leukemia in a case with t(12;17)(p13;q11)/TAF15-ZNF384 rearrangement. Leuk. Lymphoma. 2013.
36. Roberts K G, Morin R D, Zhang J, Hirst M, Zhao Y, Su X, et al. Genetic alterations activating kinase and cytokine receptor signaling in high-risk acute lymphoblastic leukemia. Cancer Cell [Internet]. 2012/08/18. 2012; 22:153-66. Available from: http://www.ncbi.nlm.nih.gov/entrez/query.fcgi?cmd=Retrieve&db=PubMed&dopt=Citation&list_uids=22897847
37. Chase A, Ernst T, Fiebig A, Collins A, Grand F, Erben P, et al. TFG, a target of chromosome translocations in lymphoma and soft tissue tumors, fuses to GPR128 in healthy individuals. Haematologica. 2010;
38. McClure B J, Heatley S L, Kok C H, Sadras T, An J, Hughes T P, et al. Pre-B acute lymphoblastic leukaemia recurrent fusion, EP300-ZNF384, is associated with a distinct gene expression. Br J Cancer. 2018; 118:1000-4.
39. Babiceanu M, Qin F, Xie Z, Jia Y, Lopez K, Janus N, et al. Recurrent chimeric fusion RNAs in non-cancer tissues and cells. Nucleic Acids Res. 2016;
40. Hu X, Wang Q, Tang M, Barthel F, Amin S, Yoshihara K, et al. TumorFusions: An integrative resource for cancer-associated transcript fusions. Nucleic Acids Res. 2018;46:D1144-9.
41. Tate J G, Bamford S, Jubb H C, Sondka Z, Beare D M, Bindal N, et al. COSMIC: The Catalogue Of Somatic Mutations In Cancer. Nucleic Acids Res. 2019;
42. Jang Y E, Jang I, Kim S, Cho S, Kim D, Kim K, et al. ChimerDB 4.0: an updated and expanded database of fusion genes. Nucleic Acids Res. 2020;
43. Hancock J M, Zvelebil M J, Griffith M, Griffith O L. Mitelman Database (Chromosome Aberrations and Gene Fusions in Cancer). Dict Bioinforma Comput Biol. 2004.
44. Kim P, Zhou X. FusionGDB: Fusion gene annotation DataBase. Nucleic Acids Res. 2019;
45. Tanasi I, Ba I, Sirvent N, Braun T, Cuccuini W, Ballerini P, et al. Efficacy of tyrosine kinase inhibitors in Ph-like acute lymphoblastic leukemia harboring ABL-class rearrangements. Blood. 2019.
46. Yu W, Wang Y, Rao Q, Jiang Y, Zhang W, Li Y. Xp11.2 translocation renal neoplasm with features of TFE3 rearrangement associated renal cell carcinoma and Xp11 translocation renal mesenchymal tumor with melanocytic differentiation harboring NONO-TFE3 fusion gene. Pathol Res Pract. 2019;
47. Kloth L, Belge G, Burchardt K, Loeschke S, Wosniok W, Fu X, et al. Decrease in thyroid adenoma associated (THADA) expression is a marker of dedifferentiation of thyroid tissue. BMC Clin Pathol. 2011;
48. Kim E, Lisby A, Ma C, Lo N, Ehmer U, Hayer K E, et al. Promotion of growth factor signaling as a critical function of p-catenin during HCC progression. Nat Commun. 2019;
49. Schoeler K, Aufschnaiter A, Messner S, Derudder E, Herzog S, Villunger A, et al. TET enzymes control antibody production and shape the mutational landscape in germinal centre B cells. FEBS J. 2019;
50. Tsagaratou A, Lio C W J, Yue X, Rao A. TET methylcytosine oxidases in T cell and B cell development and function. Front. Immunol. 2017.
51. Tan L, Shi Y G. Tet family proteins and 5-hydroxymethylcytosine in development and disease. Development. 2012;
52. Hock H, Shimamura A. ETV6 in hematopoiesis and leukemia predisposition. Semin. Hematol. 2017.
53. Xu Y, Xu C, Kato A, Tempel W, Abreu J G, Bian C, et al. Tet3 CXXC domain and dioxygenase activity cooperatively regulate key genes for Xenopus eye and neural development. Cell. 2012;
54. Steeghs E M P, Boer J M, Hoogkamer A Q, Boeree A, de Haas V, de Groot-Kruseman H A, et al. Copy number alterations in B-cell development genes, drug resistance, and clinical outcome in pediatric B-cell precursor acute lymphoblastic leukemia. Sci Rep. 2019;
55. Yoshihara K, Wang Q, Torres-Garcia W, Zheng S, Vegesna R, Kim H, et al. The landscape and therapeutic relevance of cancer-associated transcript fusions. Oncogene. 2015;
56. Chiaretti S, Messina M, Foá R. BCR/ABL1-like acute lymphoblastic leukemia: How to diagnose and treat? Cancer. 2019.
57. Wang Y, Wu N, Liu D, Jin Y. Recurrent Fusion Genes in Leukemia: An Attractive Target for Diagnosis and Treatment. Curr Genomics. 2017;
58. Roberts K G, Mullighan C G. Genomics in acute lymphoblastic leukaemia: Insights and treatment implications. Nat. Rev. Clin. Oncol. 2015.
59. Roberts K G, Yang Y, Payne-turner D, Lin W, Files J K, Dickerson K, et al. Oncogenic role and therapeutic targeting of ABL-class and JAK-STAT activating kinase alterations in Ph-like ALL. Blood Adv. 2017; 1:1657-71.
60. Sarno J, Savino A M, Buracchi C, Palmi C, Pinto S, Bugarin C, et al. SRC/ABL inhibition disrupts CRLF2-driven signaling to induce cell death in B-cell acute lymphoblastic leukemia. Oncotarget. 2018;
61. Tasian S K, Doral M Y, Borowitz M J, Wood B L, Chen I M, Harvey R C, et al. Aberrant STAT5 and PI3K/mTOR pathway signaling occurs in human CRLF2-rearranged B-precursor acute lymphoblastic leukemia. Blood. 2012;
62. Salome′, M.1; Caronni, C.1; Runfola, V.1; Giambruno, R.1; Campolungo, D.1; Ghirardi, C.1; Gabellini D. Characterization of a DUX4-IGH inhibitor as a possible treatment for acute lymphoblastic leukemia. HemaSphere [Internet]. 2019; 3:768. Available from: https://journals.lww.com/hemasphere/FullText/2019/06001/CHARACTERIZATION_OF_A_DUX4_IGH_INHIBITOR_AS_A.1539.aspx
63. Wang X, Qiao Y, Asangani I A, Ateeq B, Poliakov A, Cieslik M, et al. Development of Peptidomimetic Inhibitors of the ERG Gene Fusion Product in Prostate Cancer. Cancer Cell. 2017;
64. Debnath B, Xu S, Grande F, Garofalo A, Neamati N. Small molecule inhibitors of CXCR4. Theranostics. 2013.
65. Gaur P, Verma V, Gupta S, Sorani E, Vainstein Haras A, Oberkovitz G, et al. CXCR4 antagonist (BL-8040) to enhance antitumor effects by increasing tumor infiltration of antigen-specific effector T-cells. J Clin Oncol. 2018;
66. Hidalgo M M, Epelbaum R, Semenisty V, Geva R, Golan T, Borazanci E H, et al. Evaluation of pharmacodynamic (PD) biomarkers in patients with metastatic pancreatic cancer treated with BL-8040, a novel CXCR4 antagonist. J Clin Oncol. 2018;
67. Beider K, Darash-Yahana M, Blaier O, Koren-Michowitz M, Abraham M, Wald H, et al. Combination of imatinib with CXCR4 Antagonist BKT140 overcomes the protective effect of stroma and targets CML in vitro and in vivo. Mol Cancer Ther. 2014;
68. Amer M. Zeidan, Pamela Becker, Alexander I. Spira, Prapti A. Patel, Gary J. Schiller, Michaela L. Tsai, Tara L. Lin, Maya Ridinger, Mark Erlander SLS and JEC. Phase Ib safety, preliminary anti-leukemic activity and biomarker analysis of the polo-like kinase 1 (PLK1) inhibitor, onvansertib, in combination with low-dose cytarabine or decitabine in patients with relapsed or refractory acute myeloid leukemia. 2019.
69. Cho B S, Kim H J, Konopleva M. Targeting the CXCL12/CXCR4 axis in acute myeloid leukemia: From bench to bedside. Korean J. Intern. Med. 2017.
70. Tsaouli G, Ferretti E, Bellavia D, Vacca A, Felli M P. Notch/CXCR4 partnership in acute lymphoblastic leukemia progression. J. Immunol. Res. 2019.
71. Goossens S, Van Vlierberghe P. Overcoming Steroid Resistance in T Cell Acute Lymphoblastic Leukemia. PLoS Med. 2016;
72. Mulero M C, Aubareda A, Orzáez A, Messeguer J, Serrano-Candelas E, Martinez-Hoyer S, et al. Inhibiting the calcineurin-NFAT (nuclear factor of activated T cells) signaling pathway with a regulator of calcineurin-derived peptide without affecting general calcineurin phosphatase activity. J Biol Chem. 2009;
73. Kume K, Ikeda M, Miura S, Ito K, Sato K A, Ohmori Y, et al. a-Amanitin Restrains Cancer Relapse from Drug-Tolerant Cell Subpopulations via TAF15. Sci Rep. 2016;
74. Qian M, Zhang H, Kham S K Y, Liu S, Jiang C, Zhao X, et al. Whole-transcriptome sequencing identifies a distinct subtype of acute lymphoblastic leukemia with predominant genomic abnormalities of EP300 and CREBBP. Genome Res. 2017;
75. Inokuchi K, Wakita S, Hirakawa T, Tamai H, Yokose N, Yamaguchi H, et al. RCSD1-ABL1-positive B lymphoblastic leukemia is sensitive to dexamethasone and tyrosine kinase inhibitors and rapidly evolves clonally by chromosomal translocations. Int J Hematol. 2011;
76. Frech M, Jehn L B, Stabla K, Mielke S, Steffen B, Einsele H, et al. Dasatinib and allogeneic stem cell transplantation enable sustained response in an elderly patient with RCSD1-ABL1-positive acute lymphoblastic leukemia. Haematologica. 2017.
77. Moorman A V, Moorman A. New and emerging prognostic and predictive genetic biomarkers in B-cell precursor acute lymphoblastic leukemia. Hematology Am Soc Hematol Educ Program. 2015; 9:7-16.
78. Bernt K M, Armstrong S A. Targeting epigenetic programs in MLL-rearranged leukemias. Hematology Am. Soc. Hematol. Educ. Program. 2011.
79. Campbell C T, Haladyna J N, Drubin D A, Thomson T M, Maria M J, Yamauchi T, et al. Mechanisms of pinometostat (EPZ-5676) treatment-emergent resistance in MLL-rearranged leukemia. Mol Cancer Ther. 2017;
80. Damayanti N P, Budka J A, Khella H W Z, Ferris M W, Ku S Y, Kauffman E, et al. Therapeutic targeting of TFE3/IRS-1/PI3K/mTOR axis in translocation renal cell carcinoma. Clin Cancer Res. 2018;
81. Masetti R, Bertuccio S N, Pession A, Locatelli F. CBFA2T3-GLIS2-positive acute myeloid leukaemia. A peculiar paediatric entity. Br. J. Haematol. 2019.
82. Häfliger P, Graff J, Rubin M, Stooss A, Dettmer M S, Altmann K H, et al. The LAT1 inhibitor JPH203 reduces growth of thyroid carcinoma in a fully immunocompetent mouse model. J Exp Clin Cancer Res. 2018;
83. Marson C M, Matthews C J, Atkinson S J, Lamadema N, Thomas N S B. Potent and Selective Inhibitors of Histone Deacetylase-3 Containing Chiral Oxazoline Capping Groups and a N-(2-Aminophenyl)-benzamide Binding Unit. J Med Chem. 2015;
84. Lin Y Te, Liu Y C, Chao C C K. Inhibition of JNK and prothymosin-alpha sensitizes hepatocellular carcinoma cells to cisplatin. Biochem Pharmacol. 2016;
85. Lin Y Te, Lu H P, Chao C C K. Oncogenic c-Myc and prothymosin-alpha protect hepatocellular carcinoma cells against sorafenib-induced apoptosis. Biochem Pharmacol. 2015;
86. Miyata H, Satouh Y, Mashiko D, Muto M, Nozawa K, Shiba K, et al. Sperm calcineurin inhibition prevents mouse fertility with implications for male contraceptive. Science (80-). 2015;
87. Koppaka V, Thompson D C, Chen Y, Ellermann M, Nicolaou K C, Juvonen R O, et al. Aldehyde dehydrogenase inhibitors: A comprehensive review of the pharmacology, mechanism of action, substrate specificity, and clinical application. Pharmacol Rev. 2012;
88. Bouche L, Christ C D, Siegel S, Fernandez-Montalvan A E, Holton S J, Fedorov O, et al. Benzoisoquinolinediones as Potent and Selective Inhibitors of BRPF2 and TAF1/TAF1L Bromodomains. J Med Chem. 2017;
89. Jessen K, Moseley E, Chung E Y L, Otuski L, Tarantelli C, Gaudio E, et al. TK216, a Novel, Small Molecule Inhibitor of the ETS-Family of Transcription Factors, Displays Anti-Tumor Activity in AML and DLBCL. Blood. 2016;
90. Han H, Jain A D, Truica M I, Izquierdo-Ferrer J, Anker J F, Lysy B, et al. Small-Molecule MYC Inhibitors Suppress Tumor Growth and Enhance Immunotherapy. Cancer Cell. 2019;
91. Pourdehnad M, Truitt M L, Siddiqi I N, Ducker G S, Shokat K M, Ruggero D. Myc and mTOR converge on a common node in protein synthesis control that confers synthetic lethality in Myc-driven cancers. Proc Natl Acad Sci USA. 2013; 110:11988-93.
92. Jin X, Zhu L, Cui Z, Tang J, Xie M, Ren G. Elevated expression of GNAS promotes breast cancer cell proliferation and migration via the PI3K/AKT/Snail1/E-cadherin axis. Clin Transl Oncol. 2019;
93. Paz K, Flynn R, Du J, Qi J, Luznik L, Maillard I, et al. Small-molecule BCL6 inhibitor effectively treats mice with nonsclerodermatous chronic graft-versus-host disease. Blood. 2019;
94. Fan J, Tian L, Li M, Huang S H, Zhang J, Zhao B. MARCH8 is associated with poor prognosis in non-small cell lung cancers patients. Oncotarget. 2017; 8:108238-48.
95. Toska E, Castel P, Chhangawala S, Arruabarrena-Aristorena A, Chan C, Hristidis V C, et al. PI3K Inhibition Activates SGK1 via a Feedback Loop to Promote Chromatin-Based Regulation of ER-Dependent Gene Expression. Cell Rep. 2019;
96. Britten O, Ragusa D, Tosi S, Kamel Y M. MLL-Rearranged Acute Leukemia with t(4,11)(q21,q23)-Current Treatment Options. Is There a Role for CAR-T Cell Therapy? Cells. 2019. doi:10.3390/cells8111341.
97. Reshmi S C, Harvey R C, Roberts K G, Stonerock E, Smith A, Jenkins H et al. Targetable kinase gene fusions in high-risk B-ALL: a study from the Children's Oncology Group. Blood 2017, 129: 3352-3362.

1. A method to identify at least one genetic fusion in the genome of a subject affected by a disease comprising the following steps:
a) obtaining genomic raw sequencing data from a sample isolated from the subject,
b) analyzing said data with at least three informatic tools able to identify genetic fusions from said genomic sequencing data thereby obtaining a first genetic fusion list comprising fusions identified by at least one of said tools,
c) selecting genetic fusions from said first genetic fusion list, being detected by at least three of said tools used in step b) thereby obtaining a second genetic fusion list,
d) selecting genetic fusions from said first genetic fusion list being detected by one or two of said tools used in step b) and adding them to said second genetic fusion list provided that they meet at least one of the following criteria:
d1. genetic fusions are known for said disease,
d2. for fusions detected by two different tools but not known for said disease, fusion is not marked “false positive” by anyone of said tools used in b),
d3. for fusions detected by two tools, the fusion is labeled as significant for at least one of the three following events: a) positive score in a tool combined with read/s positivity in the other tool, b) fusion positive comments in the output of a tool, c) EBF1 and ERG genes read-throughs,
and optionally
e) comparing the fusions present in said obtained second genetic fusion list to at least one database of genetic fusions in order to obtain an annotated fusion list wherein for each fusion it is annotated if said fusion is known in other diseases and/or in normal samples.

2. The method according to claim 1 wherein in step b) said informatic tools are selected from the group consisting of: Fusion Catcher, STAR-Fusion, RNA-Seq Alignment and TopHat Alignment.

3. The method according to claim 1 wherein in step e) said genetic fusion database is selected from: i) tumor fusion gene data portal (https://www.tumorfusions.org/), ii) COSMIC (https://cancer.sanger.ac.uk/cosmic/fusion), iii) ChimerKB (http://www.kobic.re.kr/chimerdb/chimerkb), iv) Mitelman Database of Chromosome Aberrations and Gene Fusions in Cancer (https://mitelmandatabase.isb-cgc.org/mb_search), v) Fusion Gene annotation DataBase (https://ccsm.uth.edu/FusionGDB/).

4. The method according to claim 1 comprising the following steps:
a) obtaining genomic raw sequencing data from a sample isolated from the subject,
b) analyzing said data with the following tools: Fusion Catcher, STAR-Fusion, RNA-Seq Alignment and TopHat Alignment, thereby obtaining a first genetic fusion list comprising fusions identified by at least one of said tools,
c) selecting genetic fusions from said first genetic fusion list, being detected by at least three of tools as in b) thereby obtaining a second genetic fusion list,
d) selecting genetic fusions from said first genetic fusion list being detected by one or two of tools as in b) and adding them to said second genetic fusion list provided that they meet at least one of the following criteria:
d1. genetic fusions are known for said disease,
d2. for fusions detected by two different tools but not known for said disease, fusion is not marked “false positive” by the tool Fusion Catcher,
d3. for fusions detected by two tools, the fusion is labeled as significant for at least one of the three following events: a) Manta positive score combined with read/s positivity in the other tool, b) fusion positive comments in “FusionCatcher summary candidate fusion” output, c) EBF1 and ERG genes read-throughs in FusionCatcher,
and optionally
e) comparing the fusions present in said obtained second genetic fusion list to at least one genetic fusion database selected from i) tumor fusion gene data portal (https://www.tumorfusions.org/), ii) COSMIC (https://cancer.sanger.ac.uk/cosmic/fusion), iii) ChimerKB (http://www.kobic.re.kr/chimerdb/chimerkb), iv) Mitelman Database of Chromosome Aberrations and Gene Fusions in Cancer (https://mitelmandatabase.isb-cgc.org/mb_search), v) Fusion Gene annotation DataBase (https://ccsm.uth.edu/FusionGDB/), in order to obtain an annotated fusion list wherein for each fusion it is annotated if said fusion is known in other diseases and/or in normal samples.

5. The method according to claim 1 wherein genomic raw sequencing data obtained in step a) are converted to FASTQ file format.

6. The method according to claim 1 wherein in step d) fusions are selected and added to said second fusion list if criteria d1, d2 and d3 are all satisfied.

7. The method according to claim 6 wherein criteria d1, d2 and d3 are considered in the following order: d1 as first, d2 as second and d3 as third criteria.

8. The method according to claim 1 wherein in step d1) the database of FIG. 22 is used.

9. The method according to claim 1 wherein said subject is affected by a cancer.

10. The method according to claim 9 wherein said cancer is a solid cancer or a hematological cancer.

11. The method according to claim 10 wherein said subject is affected by acute myeloid leukaemia or acute lymphoblastic leukaemia.

12. The method according to claim 10 wherein said haematological tumor is selected from: T-Cell Lymphoblastic Leukemia (T-ALL), B-Cell Lymphoblastic Lymphoma, T-Cell Lymphoblastic Lymphoma, High grade Lymphoma, Lympho/myeloid acute leukemia and myeloid leukemias, acute myeloid leukemia, essential thrombocythemia, myelodysplastic syndrome, and hypereosinophilic syndrome.

13. The method according to claim 10 wherein said solid tumor is selected from: esophageal carcinoma, sarcomas, lynch syndrome, skin carcinoma and breast cancer.

14. The method according to claim 9 wherein said subject is affected by B-cell acute lymphoblastic leukemia (B-ALL).

15. The method according to claim 14 wherein said subject is affected by a B-cell acute lymphoblastic leukemia (B-ALL) classified according to at least one of the following genomic alterations: t(1,19), t(4,11), t(9,22)/Ph+, Ph−/−/−.

16. The method according to claim 14 wherein in step d1) the database of FIG. 22 is used.

17. The method according to claim 1 which is repeated one or more times on the same subject to evaluate progression of the disease.

18. A method to classify a subject affected by a disease into a known subtype or subgroup or subclass of said disease comprising using the method of claim 1.

19.-21. (canceled)

22. A method to select a therapeutic treatment for an adult B-cell acute lymphoblastic leukemia (B-ALL) subject that is negative for t(9;22), t(4;11) and t(1;19) translocations (Ph−/−/− B-ALL subject) comprising detecting in a sample of the subject the presence of at least one genetic fusion selected from the group consisting of the fusions indicated in any one of the tables 1, 4 or 5 or in FIG. 23.

23.-25. (canceled)

26. A method of treating and/or preventing B-cell acute lymphoblastic leukemia in a subject, the method comprising:
a) obtaining genomic raw sequencing data from a sample isolated from the subject,
b) analyzing said data with at least three informatic tools able to identify genetic fusions from said genomic sequencing data thereby obtaining a first genetic fusion list comprising fusions identified by at least one of said tools,
c) selecting genetic fusions from said first genetic fusion list, being detected by at least three of said tools used in step b) thereby obtaining a second genetic fusion list,
d) selecting genetic fusions from said first genetic fusion list being detected by one or two of said tools used in step b) and adding them to said second genetic fusion list provided that they meet at least one of the following criteria:
d1. genetic fusions are known for said disease,
d2. for fusions detected by two different tools but not known for said disease, fusion is not marked “false positive” by anyone of said tools used in b),
d3. for fusions detected by two tools, the fusion is labeled as significant for at least one of the three following events: a) positive score in a tool combined with read/s positivity in the other tool, b) fusion positive comments in the output of a tool, c) EBF1 and ERG genes read-throughs.
e) administering to the subject at least one inhibitor reported in table 5 to be suitable for the second genetic fusion identified in step d.


SEQUENCE LISTING (62 sequences)

SEQ ID NO: 1 - DNA, 21 bp, Artificial Sequence
  Primer RUNX1 ex3F
ggaaaagcttcactctgacca

SEQ ID NO: 2 - DNA, 20 bp, Artificial Sequence
  Primer RCAN1 ex2R
ggagaaggggttgctgaagt

SEQ ID NO: 3 - DNA, 23 bp, Artificial Sequence
  Primer PAG1 ex2F
ttttctcctatttcagcagttgg

SEQ ID NO: 4 - DNA, 22 bp, Artificial Sequence
  Primer PLAG1 ex3R
actttgatcttagccagtccca

SEQ ID NO: 5 - DNA, 15 bp, Artificial Sequence
  Primer PAG1 ex1F
cggagtttgcagagg

SEQ ID NO: 6 - DNA, 22 bp, Artificial Sequence
  Primer PLAG1 ex3R
actttgatcttagccagtccca

SEQ ID NO: 7 - DNA, 21 bp, Artificial Sequence
  Primer PPP3CC ex3
ggatcacctagtaacacacgc

SEQ ID NO: 8 - DNA, 18 bp, Artificial Sequence
  Primer CCAR2 ex2R
ctgagaagttgcgtcccc

SEQ ID NO: 9 - DNA, 21 bp, Artificial Sequence
  Primer MARCH8 ex1UTR
ggaagctcggactagtgatcc

SEQ ID NO: 10 - DNA, 19 bp, Artificial Sequence
  Primer NCOA4 ex4R
aagacattccaggtgacgg

SEQ ID NO: 11 - DNA, 20 bp, Artificial Sequence
  Primer NUMA1 ex26F
cctcaacacacccaagaagc

SEQ ID NO: 12 - DNA, 20 bp, Artificial Sequence
  Primer CSF1R ex12R
tgccctcatagctctcgatg

SEQ ID NO: 13 - DNA, 21 bp, Artificial Sequence
  Primer IKZF1 ex7F
acagtgaaatggcagaagacc

SEQ ID NO: 14 - DNA, 20 bp, Artificial Sequence
  Primer IGKV5-2 ex2R
cgctgacatgaatgctggag

SEQ ID NO: 15 - DNA, 20 bp, Artificial Sequence
  Primer CYFIP2 ex5F
gcttgccccgacatgagtat

SEQ ID NO: 16 - DNA, 18 bp, Artificial Sequence
  Primer EBF1 ex7R
ccgcatgtcacgtgggtt

SEQ ID NO: 17 - DNA, 20 bp, Artificial Sequence
  Primer UBXN4 ex1F
gagactacacaccgagcgag

SEQ ID NO: 18 - DNA, 20 bp, Artificial Sequence
  Primer CXCR4 ex2R
tcttcacggaaacagggttc

SEQ ID NO: 19 - DNA, 20 bp, Artificial Sequence
  Primer ZFPM1 ex1F
cgcgggttccattgagaaaa

SEQ ID NO: 20 - DNA, 20 bp, Artificial Sequence
  Primer SLC7A5 ex3R
aatgccagcacaatgttccc

SEQ ID NO: 21 - DNA, 19 bp, Artificial Sequence
  Primer CBFA2T3 ex1F
atgccggcttcaagactga

SEQ ID NO: 22 - DNA, 20 bp, Artificial Sequence
  Primer SLC7A5 ex3R
aatgccagcacaatgttccc

SEQ ID NO: 23 - DNA, 20 bp, Artificial Sequence
  Primer NCOR2 ex1F
gagtctttgaggacacagcc

SEQ ID NO: 24 - DNA, 19 bp, Artificial Sequence
  Primer BCL7A ex3R
atcaaccttgggctccgtc

SEQ ID NO: 25 - DNA, 21 bp, Artificial Sequence
  Primer NCOR2 ex17F
tccatggagctgaatgagagt

SEQ ID NO: 26 - DNA, 19 bp, Artificial Sequence
  Primer BCL7A ex3R
atcaaccttgggctccgtc

SEQ ID NO: 27 - DNA, 27 bp, Artificial Sequence
  Primer TAF15_ex6F2
agagcaccttcctatgaccagccagac

SEQ ID NO: 28 - DNA, 25 bp, Artificial Sequence
  Primer ZNF384_ex4R3
gccagaccacagcccttctctggca

SEQ ID NO: 29 - DNA, 21 bp, Artificial Sequence
  Primer PAX5 ex9F
ggagtccctacagccaccctc

SEQ ID NO: 30 - DNA, 25 bp, Artificial Sequence
  Primer ZCCHC7e5R2
gggggctggacaggaatacaggaga

SEQ ID NO: 31 - DNA, 23 bp, Artificial Sequence
  Primer ZEB2 ex2F
tcttatcaatgaagcagccgatc

SEQ ID NO: 32 - DNA, 20 bp, Artificial Sequence
  Primer CXCR4 ex2R
gagtagatggtgggcaggaa

SEQ ID NO: 33 - DNA, 20 bp, Artificial Sequence
  Primer ZEB2 ex1UTRF
agctgtttcttcgcttccac

SEQ ID NO: 34 - DNA, 20 bp, Artificial Sequence
  Primer CXCR4 ex2R
gagtagatggtgggcaggaa

SEQ ID NO: 35 - DNA, 20 bp, Artificial Sequence
  Primer IL7R_ex6F
tgcatggctactgaatgctc

SEQ ID NO: 36 - DNA, 20 bp, Artificial Sequence
  Primer IL7R_ex6R
ggacagcgtttgcctaatgt

SEQ ID NO: 37 - DNA, 20 bp, Artificial Sequence
  Primer CRLF2_ex6F
cgcacgtcatgttgaaaact

SEQ ID NO: 38 - DNA, 21 bp, Artificial Sequence
  Primer CRLF2_ex6R
ccatcataagagtgggcattg

SEQ ID NO: 39 - DNA, 18 bp, Artificial Sequence
  Primer JAK2_ex16F
ctcaatgcatgcctccaa

SEQ ID NO: 40 - DNA, 19 bp, Artificial Sequence
  Primer JAK2_ex16R
acaacatgccctttacacc

SEQ ID NO: 41 - DNA, 20 bp, Artificial Sequence
  Primer RAB3IP ex3 F
acgaagcccatctgttttgg

SEQ ID NO: 42 - DNA, 20 bp, Artificial Sequence
  Primer HMGA2 ex5 R
gtcctcttcggcagactctt

SEQ ID NO: 43 - DNA, 20 bp, Artificial Sequence
  Primer MSI2 ex7-8 F
actaccaacaggcacagagg

SEQ ID NO: 44 - DNA, 20 bp, Artificial Sequence
  Primer C17orf64 ex6 R
ctctggagtttctggggctt

SEQ ID NO: 45 - DNA, 18 bp, Artificial Sequence
  Primer TCF3 ex16 F
cctcatgcacaaccacgc

SEQ ID NO: 46 - DNA, 20 bp, Artificial Sequence
  Primer HLF ex4 R
ctccttcctcaagtcagcca

SEQ ID NO: 47 - DNA, 18 bp, Artificial Sequence
  Primer PLCL1 ex1 F
gatgagggaccgtcgcag

SEQ ID NO: 48 - DNA, 23 bp, Artificial Sequence
  Primer CDK1 ex3 R
caactccataggtaccttctcca

SEQ ID NO: 49 - DNA, 19 bp, Artificial Sequence
  Primer BANP ex6 F
ggactacctcttccaccgc

SEQ ID NO: 50 - DNA, 20 bp, Artificial Sequence
  Primer SLC7A5 ex3R
aatgccagcacaatgttccc

SEQ ID NO: 51 - DNA, 19 bp, Artificial Sequence
  Primer ETV6 ex1 F
ccgggagagatgctggaag

SEQ ID NO: 52 - DNA, 21 bp, Artificial Sequence
  Primer RP11-434C1.2 ex2 R
agctagattggttcctggtga

SEQ ID NO: 53 - DNA, 19 bp, Artificial Sequence
  Primer ETV6 ex1 F
ccgggagagatgctggaag

SEQ ID NO: 54 - DNA, 19 bp, Artificial Sequence
  Primer CCND2 ex5 R
ttggtcctgacggtactgc

SEQ ID NO: 55 - DNA, 20 bp, Artificial Sequence
  Primer CCND2 ex4 F
tgtacccaccgtcgatgatc

SEQ ID NO: 56 - DNA, 20 bp, Artificial Sequence
  Primer ETV6 ex2 R
gaacatgaagtggcgtcgag

SEQ ID NO: 57 - DNA, 20 bp, Artificial Sequence
  Primer BCR ex1 F
gaactcgcaacagtccttcg

SEQ ID NO: 58 - DNA, 20 bp, Artificial Sequence
  Primer MAPK1 ex2 R
taggtctggtgctcaaaggg

SEQ ID NO: 59 - DNA, 19 bp, Artificial Sequence
  Primer CD74 ex6 F
gatgcaccattggctcctg

SEQ ID NO: 60 - DNA, 21 bp, Artificial Sequence
  Primer CAMK2A ex2 R
tgtgttgatgatcttggcagc

SEQ ID NO: 61 - DNA, 20 bp, Artificial Sequence
  Primer PAX5 ex6 F
cggggagacttgttcacaca

SEQ ID NO: 62 - DNA, 20 bp, Artificial Sequence
  Primer MLLT3/AF9 ex2 R
atgttactgtgctccggacc